SEQUENCE LISTING (73 sequences)

SEQ ID NO: 1 - protein, 9 aa, Artificial Sequence
  Synthetic
GGISRYKTM

SEQ ID NO: 2 - protein, 14 aa, Artificial Sequence
  Synthetic
EFVAGITWGGSTYY

SEQ ID NO: 3 - protein, 13 aa, Artificial Sequence
  Synthetic
SVDGGTYADPYHY

SEQ ID NO: 4 - protein, 120 aa, Artificial Sequence
  Synthetic
QVQLQESGGGLVQAGGSLRLSCAASGGISRYKTMGWYRQAPGKEREFVAGITWGGSTYYADSVKGRFTISRDNAKNTVYLQMNSLKPEDTAVYYCSVDGGTYADPYHYYWGQGTQVTVSS

SEQ ID NO: 5 - protein, 9 aa, Artificial Sequence
  Synthetic
PGIYRYKTI

SEQ ID NO: 6 - protein, 14 aa, Artificial Sequence
  Synthetic
SFVAAITWGGLTYR

SEQ ID NO: 7 - protein, 13 aa, Artificial Sequence
  Synthetic
SVDGGTRAQPVHY

SEQ ID NO: 8 - protein, 120 aa, Artificial Sequence
  Synthetic
QVQLQESGGGLVQAGGSLRLSCAASPGIYRYKTIAWYRQAPGKERSFVAAITWGGLTYRADSVKGRFTVSRDNAKNTVYLQMNSLKPEDTAVYYCSVDGGTRAQPVHYYWGQGTQVTVSS

SEQ ID NO: 9 - protein, 9 aa, Artificial Sequence
  Synthetic
GYISRYHTM

SEQ ID NO: 10 - protein, 14 aa, Artificial Sequence
  Synthetic
EFVAGITWGGSTYY

SEQ ID NO: 11 - protein, 13 aa, Artificial Sequence
  Synthetic
AVDGGTYADPYHY

SEQ ID NO: 12 - protein, 120 aa, Artificial Sequence
  Synthetic
QVQLQESGGGLVQAGGSLRLSCAASGYISRYHTMGWYRQAPGKEREFVAGITWGGSTYYADSVKGRFTISRDNAKNTVYLQMNSLKPEDTAVYYCAVDGGTYADPYHYYWGQGTQVTVSS

SEQ ID NO: 13 - protein, 9 aa, Artificial Sequence
  Synthetic
PYISRYHTM

SEQ ID NO: 14 - protein, 14 aa, Artificial Sequence
  Synthetic
EFVAAITWGGSTYY

SEQ ID NO: 15 - protein, 13 aa, Artificial Sequence
  Synthetic
AVDGGTYADPYHY

SEQ ID NO: 16 - protein, 120 aa, Artificial Sequence
  Synthetic
QVQLQESGGGLVQAGGSLRLSCAASPYISRYHTMGWYRQAPGKEREFVAAITWGGSTYYADSVKGRFTISRDNAKNTVYLQMNSLKPEDTAVYYCAVDGGTYADPYHYYWGQGTQVTVSS

SEQ ID NO: 17 - protein, 9 aa, Artificial Sequence
  Synthetic
SYISRYHTM

SEQ ID NO: 18 - protein, 14 aa, Artificial Sequence
  Synthetic
SFVAGITWGGLTYY

SEQ ID NO: 19 - protein, 13 aa, Artificial Sequence
  Synthetic
AVDGGTRADPYHY

SEQ ID NO: 20 - protein, 120 aa, Artificial Sequence
  Synthetic
QVQLQESGGGLVQAGGSLRLSCAASSYISRYHTMGWYRQAPGKERSFVAGITWGGLTYYADSVKGRFTVSRDNAKNTVYLQMNSLKPEDTAVYYCAVDGGTRADPYHYYWGQGTQVTVSS

SEQ ID NO: 21 - protein, 9 aa, Artificial Sequence
  Synthetic
PGISRYKTM

SEQ ID NO: 22 - protein, 14 aa, Artificial Sequence
  Synthetic
SFVAAITWGGLTYY

SEQ ID NO: 23 - protein, 13 aa, Artificial Sequence
  Synthetic
SVDGGTRADPYHY

SEQ ID NO: 24 - protein, 120 aa, Artificial Sequence
  Synthetic
QVQLQESGGGLVQAGGSLRLSCAASPGISRYKTMGWYRQAPGKERSFVAAITWGGLTYYADSVKGRFTVSRDNAKNTVYLQMNSLKPEDTAVYYCSVDGGTRADPYHYYWGQGTQVTVSS

SEQ ID NO: 25 - protein, 9 aa, Artificial Sequence
  Synthetic
GNISADRYM

SEQ ID NO: 26 - protein, 14 aa, Artificial Sequence
  Synthetic
EFVAAIGYGGTTYY

SEQ ID NO: 27 - protein, 12 aa, Artificial Sequence
  Synthetic
AVVDGAHSRHRY

SEQ ID NO: 28 - protein, 118 aa, Artificial Sequence
  Synthetic
QVQLQESGGGLVQAGGSLRLSCAASGNISADRYMGWYRQAPGKEREFVAAIGYGGTTYYADSVKGRFTISRDNAKNTVYLQMNSLKPEDTAVYYCAVVDGAHSRHRYWGQGTQVTVSS

SEQ ID NO: 29 - protein, 9 aa, Artificial Sequence
  Synthetic
GTISYGYVM

SEQ ID NO: 30 - protein, 14 aa, Artificial Sequence
  Synthetic
ELVAGINRGSSTYY

SEQ ID NO: 31 - protein, 16 aa, Artificial Sequence
  Synthetic
AASGDWYDWRSRYFLY

SEQ ID NO: 32 - protein, 122 aa, Artificial Sequence
  Synthetic
QVQLQESGGGLVQAGGSLRLSCAASGTISYGYVMGWYRQAPGKERELVAGINRGSSTYYADSVKGRFTISRDNAKNTVYLQMNSLKPEDTAVYYCAASGDWYDWRSRYFLYWGQGTQVTVSS

SEQ ID NO: 33 - protein, 9 aa, Artificial Sequence
  Synthetic
GSISPLYNM

SEQ ID NO: 34 - protein, 14 aa, Artificial Sequence
  Synthetic
EFVAGINSGSTTYY

SEQ ID NO: 35 - protein, 12 aa, Artificial Sequence
  Synthetic
AAYTDGYEGLDY

SEQ ID NO: 36 - protein, 118 aa, Artificial Sequence
  Synthetic
QVQLQESGGGLVQAGGSLRLSCAASGSISPLYNMGWYRQAPGKEREFVAGINSGSTTYYADSVKGRFTISRDNAKNTVYLQMNSLKPEDTAVYYCAAYTDGYEGLDYWGQGTQVTVSS

SEQ ID NO: 37 - DNA, 360 bp, Artificial Sequence
  Synthetic
caagtacaactgcaagagtctggaggtggacttgtccaggcaggcggctcgcttcgtctttcttgcgctgctagtggcgggatcagccgctataaaacaatgggatggtatcgtcaagcgccaggcaaagaacgtgaatttgtagctggaattacctgggggggatctacatattacgctgactctgtcaaaggccgtttcactatcagccgtgacaacgcaaaaaataccgtatatttgcaaatgaattcactgaaacccgaagacacagcggtgtattattgctccgttgacggggggacctacgctgacccataccattactactgggggcaagggacccaggtaacagtgtcctcc

SEQ ID NO: 38 - DNA, 360 bp, Artificial Sequence
  Synthetic
caggtccagttacaagagtcaggcggcggcttggttcaagccggcggcagtctgcgtttatcgtgtgccgcatcccctgggatttaccgctataaaaccatcgcctggtatcgtcaggcgcctgggaaagaacgcagctttgttgctgcaatcacatggggagggttaacgtaccgcgcagattcggttaaggggcgttttaccgtgtcccgcgacaatgcaaaaaacacggtatatcttcagatgaactcgttgaaaccagaagacacagctgtttactactgctcggtcgatggtgggacacgcgcccagcctgtgcattactactggggccagggtacgcaggttacagtgtcgtct

SEQ ID NO: 39 - DNA, 360 bp, Artificial Sequence
  Synthetic
caggtccagttacaggagtctgggggcggcttagtccaggccggagggagcttgcgcttgtcttgtgcagcttcgggctatatttcacgctatcacacaatgggatggtatcgccaagcacctggaaaagaacgtgaatttgtcgctgggatcacctggggtggatctacctattatgctgacagtgtcaaggggcgcttcacgatctcgcgcgacaacgcaaaaaacacggtttacctgcaaatgaacagtcttaaaccagaggatacagccgtatattactgtgcagtggacggaggtacttatgctgacccttaccattactattggggacaaggaacccaggtaactgtatccagc

SEQ ID NO: 40 - DNA, 360 bp, Artificial Sequence
  Synthetic
caagtgcagcttcaagagtcgggcggaggtttagtacaagcagggggctcgctgcgcctttcatgtgcggcaagtccctacattagccgctatcacacgatgggatggtatcgccaagcgccaggcaaagaacgcgagttcgttgcagccattacctggggaggcagcacctactatgctgatagcgtaaagggccgtttcacgatctcccgtgataacgccaaaaacacggtgtatttgcagatgaattctcttaaaccggaggatactgctgtatattactgcgccgtggacgggggaacgtatgccgacccctatcactattattggggacaaggtacgcaagttactgtttctagc

SEQ ID NO: 41 - DNA, 360 bp, Artificial Sequence
  Synthetic
caggtacaattgcaagagtctggaggcggcctggtccaggcaggaggtagtttacgcttatcttgtgccgcgtcgtcctacattagccgttatcacacaatgggatggtaccgtcaagcaccagggaaagagcgttcctttgttgctggcatcacctggggtggcttaacttactatgcagatagtgtcaaggggcgtttcacggtaagtcgtgacaatgctaagaacactgtttacttacaaatgaactcccttaaaccagaagacaccgccgtttattactgcgcggtggacggcggcacccgtgccgatccttaccattattactgggggcaggggacacaagtaacggtaagtagt

SEQ ID NO: 42 - DNA, 360 bp, Artificial Sequence
  Synthetic
caagtacaactgcaagagtctggaggtggacttgtccaggcgggtggctcacttcgcctttcatgtgccgcttcacccgggatctcgcgctataagacaatgggctggtaccgccaagcacctggaaaggaacgttccttcgttgccgcaatcacctggggaggtttgacctattatgccgattctgttaaagggcgcttcacagtgtcgcgtgataacgcaaaaaatacagtgtatttgcagatgaacagtttgaagcctgaagacacggcggtttactattgcagtgtggacggtggtacccgtgccgatccgtatcactactactgggggcaagggacccaggtaacagtgtcctcc

SEQ ID NO: 43 - DNA, 354 bp, Artificial Sequence
  Synthetic
caggttcaacttcaagaatcgggaggaggactggtccaagcgggaggcagcttacgccttagttgtgctgcctctggaaatatctcggctgaccgctacatgggttggtaccgccaggcccctgggaaagagcgtgagttcgtggctgcaatcggatacggcggaaccacttattatgctgacagtgttaagggacgtttcactatctcgcgcgataatgctaagaatacagtgtaccttcaaatgaattctcttaaaccagaggacaccgctgtttattactgtgctgttgtggacggggcgcattcacgtcatcgttactggggacaaggtacgcaggttaccgtaagtagc

SEQ ID NO: 44 - DNA, 366 bp, Artificial Sequence
  Synthetic
caggtccagttacaagaatcaggcggcggactggtccaggctggaggctcccttcgtttaagctgcgccgcttcaggaacgatttcatacgggtacgtcatgggctggtaccgccaagcacctggcaaagagcgcgagcttgtcgcgggtatcaatcgcggatcttcgacgtattatgccgacagcgtcaaagggcgtttcactatctcccgcgacaacgcgaagaataccgtctacttgcaaatgaactccctgaaaccggaagacacagccgtttattattgtgcggcaagcggggactggtatgactggcgcagccgttatttcctttattggggacaaggtactcaggtcacagtttcaagc

SEQ ID NO: 45 - DNA, 354 bp, Artificial Sequence
  Synthetic
caagtgcaactgcaggaaagcggcggcggcctggtgcaggcgggcggcagcctgcgcctgagctgcgcggcgagcggctctatttctccgctgtacaacatgggctggtatcgccaggcgccgggcaaagaacgcgaatttgttgccggtattaattctggtagtactacctattatgcggatagcgtgaaaggccgctttaccattagccgcgataacgcgaaaaacaccgtgtatctgcagatgaacagcctgaaaccggaagataccgcggtgtattattgcgcggcttacactgacggttacgaaggtcttgactattggggccagggcacccaggtgaccgtgagcagc

SEQ ID NO: 46 - protein, 12 aa, Artificial Sequence
  Synthetic
GTLVTVSSASTK

SEQ ID NO: 47 - DNA, 20 bp, Artificial Sequence
  Synthetic
acagccaagggtaaatatgg

SEQ ID NO: 48 - protein, 463 aa, Artificial Sequence
  Synthetic
QVQLQESGGGLVQAGGSLRLSCAASGGISRYKTMGWYRQAPGKEREFVAGITWGGSTYYADSVKGRFTISRDNAKNTVYLQMNSLKPEDTAVYYCSVDGGTYADPYHYYWGQGTQVTVSSGGGGSGGGGSPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVM

SEQ ID NO: 49 - DNA, 1389 bp, Artificial Sequence
  Synthetic
caagtacaactgcaagagtctggaggtggacttgtccaggcaggcggctcgcttcgtctttcttgcgctgctagtggcgggatcagccgctataaaacaatgggatggtatcgtcaagcgccaggcaaagaacgtgaatttgtagctggaattacctgggggggatctacatattacgctgactctgtcaaaggccgtttcactatcagccgtgacaacgcaaaaaataccgtatatttgcaaatgaattcactgaaacccgaagacacagcggtgtattattgctccgttgacggggggacctacgctgacccataccattactactgggggcaagggacccaggtaacagtgtcctccggaggaggaggtagcggcggaggcgggtctcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatg

SEQ ID NO: 50 - protein, 450 aa, Artificial Sequence
  Synthetic
QVQLQESGGGLVQAGGSLRLSCAASGGISRYKTMGWYRQAPGKEREFVAGITWGGSTYYADSVKGRFTISRDNAKNTVYLQMNSLKPEDTAVYYCSVDGGTYADPYHYYWGQGTQVTVSSGGGGSGGGGSPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLM

SEQ ID NO: 51 - DNA, 1350 bp, Artificial Sequence
  Synthetic
caagtacaactgcaagagtctggaggtggacttgtccaggcaggcggctcgcttcgtctttcttgcgctgctagtggcgggatcagccgctataaaacaatgggatggtatcgtcaagcgccaggcaaagaacgtgaatttgtagctggaattacctgggggggatctacatattacgctgactctgtcaaaggccgtttcactatcagccgtgacaacgcaaaaaataccgtatatttgcaaatgaattcactgaaacccgaagacacagcggtgtattattgctccgttgacggggggacctacgctgacccataccattactactgggggcaagggacccaggtaacagtgtcctccggaggaggaggtagcggcggaggcgggtctccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatg

SEQ ID NO: 52 - protein, 1095 aa, Artificial Sequence
  Synthetic
QVQLQESGGGLVQAGGSLRLSCAASGGISRYKTMGWYRQAPGKEREFVAGITWGGSTYYADSVKGRFTISRDNAKNTVYLQMNSLKPEDTAVYYCSVDGGTYADPYHYYWGQGTQVTVSSGGGGSGGGGSGGGGSMEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 53 - DNA, 3285 bp, Artificial Sequence
  Synthetic
caagtacaactgcaagagtctggaggtggacttgtccaggcaggcggctcgcttcgtctttcttgcgctgctagtggcgggatcagccgctataaaacaatgggatggtatcgtcaagcgccaggcaaagaacgtgaatttgtagctggaattacctgggggggatctacatattacgctgactctgtcaaaggccgtttcactatcagccgtgacaacgcaaaaaataccgtatatttgcaaatgaattcactgaaacccgaagacacagcggtgtattattgctccgttgacggggggacctacgctgacccataccattactactgggggcaagggacccaggtaacagtgtcctccggaggaggaggtagcggaggaggaggtagcggcggaggcgggtctatggaggagaagactgttcaggttcagaaaggattaccttctatcgatagcttgcattatctgtcagagaatagcaaaaaagaatttaaagaagaactctcaaaagcggggcaagaatctcaaaaggtcaaagagatattagcaaaagctcagcaggcagataaacaagctcaagaacttgccaaaatgaaaattcctgagaaaataccgatgaaaccgttacatggtcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatgctaaaagataagtcgtatgatctgattgatgagaaagatttcccagataaggctttgcgagaagctgtgatggcgcaggttggaaccagaaaaggtgatttggaacgtttcaatggcacattacgattggataatccagcgattcaaagtttagaaggtctaaataaatttaaaaaattagctcaattagacttgattggcttatctcgcattacaaagctcgaccgttctgttttacccgctaatatgaagccaggcaaagataccttggaaacagttcttgaaacctataaaaaggataacaaagaagaacctgctactatcccaccagtatctttgaaggtttctggtttaactggtctgaaagaattagatttgtcaggttttgaccgtgaaaccttggctggtcttgatgccgctactctaacgtctttagaaaaagttgatatttctggcaacaaacttgatttggctccaggaacagaaaatcgacaaatttttgatactatgctatcaactatcagcaatcatgttggaagcaatgaacaaacagtgaaatttgacaagcaaaaaccaactgggcattacccagatacctatgggaaaactagtctgcgcttaccagtggcaaatgaaaaagttgatttgcaaagccagcttttgtttgggactgtgacaaatcaaggaaccctaatcaatagcgaagcagactataaggcttaccaaaatcataaaattgctggacgtagctttgttgattcaaactatcattacaataactttaaagtttcttatgagaactataccgttaaagtaactgattccacattgggaaccactactgacaaaacgctagcaactgataaagaagagacctataaggttgacttctttagcccagcagataagacaaaagctgttcatactgctaaagtgattgttggtgacgaaaaaaccatgatggttaatttggcagaaggcgcaacagttattggaggaagtgctgatcctgtaaatgcaagaaaggtatttgatgggcaactgggcagtgagactgataatatctctttaggatgggattctaagcaaagtattatatttaaattgaaagaagatggattaataaagcattggcgtttcttcaatgattcagcccgaaatcctgagacaaccaataaacctattcaggaagcaagtctacaaatttttaatatcaaagattataatctagataatttgttggaaaatcccaataaatttgatgatgaaaaatattggattactgtagatacttacagtgcacaaggagagagagctactgcattcagtaatacattaaataatattactagtaaatattggcgagttgtctttgatactaaaggagatagatatagttcgccagtagtccctgaactccaaattttaggttatccgttacctaacgccgacactatcatgaaaacagtaactactgctaaagagttatctcaacaaaaagataagttttctcaaaagatgcttgatgagttaaaaataaaagagatggctttagaaacttctttgaacagtaagatttttgatgtaactgctattaatgctaatgctggagttttgaaagattgtattgagaaaaggcagctgctaaaaaaa

SEQ ID NO: 54 - protein, 937 aa, Artificial Sequence
  Synthetic
QVQLQESGGGLVQAGGSLRLSCAASGGISRYKTMGWYRQAPGKEREFVAGITWGGSTYYADSVKGRFTISRDNAKNTVYLQMNSLKPEDTAVYYCSVDGGTYADPYHYYWGQGTQVTVSSGGGGSGGGGSGGGGSMGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKDL

SEQ ID NO: 55 - DNA, 2811 bp, Artificial Sequence
  Synthetic
caagtacaactgcaagagtctggaggtggacttgtccaggcaggcggctcgcttcgtctttcttgcgctgctagtggcgggatcagccgctataaaacaatgggatggtatcgtcaagcgccaggcaaagaacgtgaatttgtagctggaattacctgggggggatctacatattacgctgactctgtcaaaggccgtttcactatcagccgtgacaacgcaaaaaataccgtatatttgcaaatgaattcactgaaacccgaagacacagcggtgtattattgctccgttgacggggggacctacgctgacccataccattactactgggggcaagggacccaggtaacagtgtcctccggaggaggaggtagcggaggaggaggtagcggcggaggcgggtctatgggaaagacagatcagcaggttggtgctaaattggtacaggaaatccgtgaaggaaaacgcggaccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatgaccgaagacaaacgctatgatgtcattgatcaaaaagacattcctgacccagcattaagagaacaaatcattcaacaagttggacagtataaaggcgatttggaacgttataacaagacattggtgcttacaggagataagattcaaaatcttaaaggactagaaaaattaagcaagttacaaaaattagagttgcgccagctatctaacgttaaagaaattactccagaacttttgccggaaagcatgaaaaaagatgctgagcttgttatggtaggcatgactggtttagaaaaactaaaccttagtggtctaaatcgtcaaactttagacggtatagacgtgaatagtattacgcatttgacatcatttgatatttcacataatagtttggacttgtcggaaaagagtgaagaccgtaaactattaatgactttgatggagcaggtttcaaatcatcaaaaaataacggtgaaaaatacggcttttgaaaatcaaaaaccgaaaggttattatcctcagacgtatgataccaaagaaggtcattatgatgttgataatgcagaacatgatattttaactgattttgtttttggaactgttactaaacgtaatacctttattggagacgaagaagcatttgctatctataaagaaggagctgtcgatggtcgacaatatgtgtctaaagactatacttatgaagcttttcgtaaagactataaaggttacaaggttcatttaactgcttctaacctaggagaaacagttacttctaaggtaactgctactactgatgaaacttacttagtagatgtttctgatggggaaaaagttgttcaccacatgaaactcaatataggatctggtgccatcatgatggaaaatctggcaaaaggggctaaagtgattggtacatctggggactttgagcaagcaaagaagattttcgatggtgaaaagtcagatagattcttcacttggggacaaactaactggatagcttttgatctaggagaaattaatcttgcgaaggaatggcgtttatttaatgcagagacaaatactgaaataaagacagatagtagcttaaacgtggctaaaggacgtcttcagattttaaaagatacaactattgatttagaaaaaatggacataaaaaatcgtaaagagtatctgtcgaatgatgaaaattggactgatgttgctcagatggatgatgcaaaagcgatatttaatagtaaattatccaatgttttatctcggtattggcggttttgtgtagatggtggagctagctcttattaccctcaatataccgaacttcaaatcctcggacaacgtttatcaaatgatgtcgctaatacgctgaaggatctg

SEQ ID NO: 56 - protein, 463 aa, Artificial Sequence
  Synthetic
QVQLQESGGGLVQAGGSLRLSCAASPGISRYKTMGWYRQAPGKERSFVAAITWGGLTYYADSVKGRFTVSRDNAKNTVYLQMNSLKPEDTAVYYCSVDGGTRADPYHYYWGQGTQVTVSSGGGGSGGGGSPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVM

SEQ ID NO: 57 - DNA, 1389 bp, Artificial Sequence
  Synthetic
caagtacaactgcaagagtctggaggtggacttgtccaggcgggtggctcacttcgcctttcatgtgccgcttcacccgggatctcgcgctataagacaatgggctggtaccgccaagcacctggaaaggaacgttccttcgttgccgcaatcacctggggaggtttgacctattatgccgattctgttaaagggcgcttcacagtgtcgcgtgataacgcaaaaaatacagtgtatttgcagatgaacagtttgaagcctgaagacacggcggtttactattgcagtgtggacggtggtacccgtgccgatccgtatcactactactgggggcaagggacccaggtaacagtgtcctccggaggaggaggtagcggcggaggcgggtctcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatg

SEQ ID NO: 58 - protein, 450 aa, Artificial Sequence
  Synthetic
QVQLQESGGGLVQAGGSLRLSCAASPGISRYKTMGWYRQAPGKERSFVAAITWGGLTYYADSVKGRFTVSRDNAKNTVYLQMNSLKPEDTAVYYCSVDGGTRADPYHYYWGQGTQVTVSSGGGGSGGGGSPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLM

SEQ ID NO: 59 - DNA, 1350 bp, Artificial Sequence
  Synthetic
caagtacaactgcaagagtctggaggtggacttgtccaggcgggtggctcacttcgcctttcatgtgccgcttcacccgggatctcgcgctataagacaatgggctggtaccgccaagcacctggaaaggaacgttccttcgttgccgcaatcacctggggaggtttgacctattatgccgattctgttaaagggcgcttcacagtgtcgcgtgataacgcaaaaaatacagtgtatttgcagatgaacagtttgaagcctgaagacacggcggtttactattgcagtgtggacggtggtacccgtgccgatccgtatcactactactgggggcaagggacccaggtaacagtgtcctccggaggaggaggtagcggcggaggcgggtctccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatg

SEQ ID NO: 60 - protein, 1090 aa, Artificial Sequence
  Synthetic
QVQLQESGGGLVQAGGSLRLSCAASPGISRYKTMGWYRQAPGKERSFVAAITWGGLTYYADSVKGRFTVSRDNAKNTVYLQMNSLKPEDTAVYYCSVDGGTRADPYHYYWGQGTQVTVSSGGGGSGGGGSMEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 61 - DNA, 3270 bp, Artificial Sequence
  Synthetic
caagtacaactgcaagagtctggaggtggacttgtccaggcgggtggctcacttcgcctttcatgtgccgcttcacccgggatctcgcgctataagacaatgggctggtaccgccaagcacctggaaaggaacgttccttcgttgccgcaatcacctggggaggtttgacctattatgccgattctgttaaagggcgcttcacagtgtcgcgtgataacgcaaaaaatacagtgtatttgcagatgaacagtttgaagcctgaagacacggcggtttactattgcagtgtggacggtggtacccgtgccgatccgtatcactactactgggggcaagggacccaggtaacagtgtcctccggaggaggaggtagcggcggaggcgggtctatggaggagaagactgttcaggttcagaaaggattaccttctatcgatagcttgcattatctgtcagagaatagcaaaaaagaatttaaagaagaactctcaaaagcggggcaagaatctcaaaaggtcaaagagatattagcaaaagctcagcaggcagataaacaagctcaagaacttgccaaaatgaaaattcctgagaaaataccgatgaaaccgttacatggtcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatgctaaaagataagtcgtatgatctgattgatgagaaagatttcccagataaggctttgcgagaagctgtgatggcgcaggttggaaccagaaaaggtgatttggaacgtttcaatggcacattacgattggataatccagcgattcaaagtttagaaggtctaaataaatttaaaaaattagctcaattagacttgattggcttatctcgcattacaaagctcgaccgttctgttttacccgctaatatgaagccaggcaaagataccttggaaacagttcttgaaacctataaaaaggataacaaagaagaacctgctactatcccaccagtatctttgaaggtttctggtttaactggtctgaaagaattagatttgtcaggttttgaccgtgaaaccttggctggtcttgatgccgctactctaacgtctttagaaaaagttgatatttctggcaacaaacttgatttggctccaggaacagaaaatcgacaaatttttgatactatgctatcaactatcagcaatcatgttggaagcaatgaacaaacagtgaaatttgacaagcaaaaaccaactgggcattacccagatacctatgggaaaactagtctgcgcttaccagtggcaaatgaaaaagttgatttgcaaagccagcttttgtttgggactgtgacaaatcaaggaaccctaatcaatagcgaagcagactataaggcttaccaaaatcataaaattgctggacgtagctttgttgattcaaactatcattacaataactttaaagtttcttatgagaactataccgttaaagtaactgattccacattgggaaccactactgacaaaacgctagcaactgataaagaagagacctataaggttgacttctttagcccagcagataagacaaaagctgttcatactgctaaagtgattgttggtgacgaaaaaaccatgatggttaatttggcagaaggcgcaacagttattggaggaagtgctgatcctgtaaatgcaagaaaggtatttgatgggcaactgggcagtgagactgataatatctctttaggatgggattctaagcaaagtattatatttaaattgaaagaagatggattaataaagcattggcgtttcttcaatgattcagcccgaaatcctgagacaaccaataaacctattcaggaagcaagtctacaaatttttaatatcaaagattataatctagataatttgttggaaaatcccaataaatttgatgatgaaaaatattggattactgtagatacttacagtgcacaaggagagagagctactgcattcagtaatacattaaataatattactagtaaatattggcgagttgtctttgatactaaaggagatagatatagttcgccagtagtccctgaactccaaattttaggttatccgttacctaacgccgacactatcatgaaaacagtaactactgctaaagagttatctcaacaaaaagataagttttctcaaaagatgcttgatgagttaaaaataaaagagatggctttagaaacttctttgaacagtaagatttttgatgtaactgctattaatgctaatgctggagttttgaaagattgtattgagaaaaggcagctgctaaaaaaa

SEQ ID NO: 62 - protein, 932 aa, Artificial Sequence
  Synthetic
QVQLQESGGGLVQAGGSLRLSCAASPGISRYKTMGWYRQAPGKERSFVAAITWGGLTYYADSVKGRFTVSRDNAKNTVYLQMNSLKPEDTAVYYCSVDGGTRADPYHYYWGQGTQVTVSSGGGGSGGGGSMGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKDL

SEQ ID NO: 63 - DNA, 2796 bp, Artificial Sequence
  Synthetic
caagtacaactgcaagagtctggaggtggacttgtccaggcgggtggctcacttcgcctttcatgtgccgcttcacccgggatctcgcgctataagacaatgggctggtaccgccaagcacctggaaaggaacgttccttcgttgccgcaatcacctggggaggtttgacctattatgccgattctgttaaagggcgcttcacagtgtcgcgtgataacgcaaaaaatacagtgtatttgcagatgaacagtttgaagcctgaagacacggcggtttactattgcagtgtggacggtggtacccgtgccgatccgtatcactactactgggggcaagggacccaggtaacagtgtcctccggaggaggaggtagcggcggaggcgggtctatgggaaagacagatcagcaggttggtgctaaattggtacaggaaatccgtgaaggaaaacgcggaccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatgaccgaagacaaacgctatgatgtcattgatcaaaaagacattcctgacccagcattaagagaacaaatcattcaacaagttggacagtataaaggcgatttggaacgttataacaagacattggtgcttacaggagataagattcaaaatcttaaaggactagaaaaattaagcaagttacaaaaattagagttgcgccagctatctaacgttaaagaaattactccagaacttttgccggaaagcatgaaaaaagatgctgagcttgttatggtaggcatgactggtttagaaaaactaaaccttagtggtctaaatcgtcaaactttagacggtatagacgtgaatagtattacgcatttgacatcatttgatatttcacataatagtttggacttgtcggaaaagagtgaagaccgtaaactattaatgactttgatggagcaggtttcaaatcatcaaaaaataacggtgaaaaatacggcttttgaaaatcaaaaaccgaaaggttattatcctcagacgtatgataccaaagaaggtcattatgatgttgataatgcagaacatgatattttaactgattttgtttttggaactgttactaaacgtaatacctttattggagacgaagaagcatttgctatctataaagaaggagctgtcgatggtcgacaatatgtgtctaaagactatacttatgaagcttttcgtaaagactataaaggttacaaggttcatttaactgcttctaacctaggagaaacagttacttctaaggtaactgctactactgatgaaacttacttagtagatgtttctgatggggaaaaagttgttcaccacatgaaactcaatataggatctggtgccatcatgatggaaaatctggcaaaaggggctaaagtgattggtacatctggggactttgagcaagcaaagaagattttcgatggtgaaaagtcagatagattcttcacttggggacaaactaactggatagcttttgatctaggagaaattaatcttgcgaaggaatggcgtttatttaatgcagagacaaatactgaaataaagacagatagtagcttaaacgtggctaaaggacgtcttcagattttaaaagatacaactattgatttagaaaaaatggacataaaaaatcgtaaagagtatctgtcgaatgatgaaaattggactgatgttgctcagatggatgatgcaaaagcgatatttaatagtaaattatccaatgttttatctcggtattggcggttttgtgtagatggtggagctagctcttattaccctcaatataccgaacttcaaatcctcggacaacgtttatcaaatgatgtcgctaatacgctgaaggatctg

SEQ ID NO: 64 - protein, 960 aa, Streptococcus pyogenes
MEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 65 - DNA, 2880 bp, Streptococcus pyogenes
atggaggagaagactgttcaggttcagaaaggattaccttctatcgatagcttgcattatctgtcagagaatagcaaaaaagaatttaaagaagaactctcaaaagcggggcaagaatctcaaaaggtcaaagagatattagcaaaagctcagcaggcagataaacaagctcaagaacttgccaaaatgaaaattcctgagaaaataccgatgaaaccgttacatggtcctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatgctaaaagataagtcgtatgatctgattgatgagaaagatttcccagataaggctttgcgagaagctgtgatggcgcaggttggaaccagaaaaggtgatttggaacgtttcaatggcacattacgattggataatccagcgattcaaagtttagaaggtctaaataaatttaaaaaattagctcaattagacttgattggcttatctcgcattacaaagctcgaccgttctgttttacccgctaatatgaagccaggcaaagataccttggaaacagttcttgaaacctataaaaaggataacaaagaagaacctgctactatcccaccagtatctttgaaggtttctggtttaactggtctgaaagaattagatttgtcaggttttgaccgtgaaaccttggctggtcttgatgccgctactctaacgtctttagaaaaagttgatatttctggcaacaaacttgatttggctccaggaacagaaaatcgacaaatttttgatactatgctatcaactatcagcaatcatgttggaagcaatgaacaaacagtgaaatttgacaagcaaaaaccaactgggcattacccagatacctatgggaaaactagtctgcgcttaccagtggcaaatgaaaaagttgatttgcaaagccagcttttgtttgggactgtgacaaatcaaggaaccctaatcaatagcgaagcagactataaggcttaccaaaatcataaaattgctggacgtagctttgttgattcaaactatcattacaataactttaaagtttcttatgagaactataccgttaaagtaactgattccacattgggaaccactactgacaaaacgctagcaactgataaagaagagacctataaggttgacttctttagcccagcagataagacaaaagctgttcatactgctaaagtgattgttggtgacgaaaaaaccatgatggttaatttggcagaaggcgcaacagttattggaggaagtgctgatcctgtaaatgcaagaaaggtatttgatgggcaactgggcagtgagactgataatatctctttaggatgggattctaagcaaagtattatatttaaattgaaagaagatggattaataaagcattggcgtttcttcaatgattcagcccgaaatcctgagacaaccaataaacctattcaggaagcaagtctacaaatttttaatatcaaagattataatctagataatttgttggaaaatcccaataaatttgatgatgaaaaatattggattactgtagatacttacagtgcacaaggagagagagctactgcattcagtaatacattaaataatattactagtaaatattggcgagttgtctttgatactaaaggagatagatatagttcgccagtagtccctgaactccaaattttaggttatccgttacctaacgccgacactatcatgaaaacagtaactactgctaaagagttatctcaacaaaaagataagttttctcaaaagatgcttgatgagttaaaaataaaagagatggctttagaaacttctttgaacagtaagatttttgatgtaactgctattaatgctaatgctggagttttgaaagattgtattgagaaaaggcagctgctaaaaaaa

SEQ ID NO: 66 - protein, 333 aa, Streptococcus pyogenes
PLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVM

SEQ ID NO: 67 - DNA, 999 bp, Streptococcus pyogenes
cctctctacggtggttactttagaacttggcatgacaaaacatcagatccaacagaaaaagacaaagttaactcgatgggagagcttcctaaagaagtagatctagcctttattttccacgattggacaaaagattatagccttttttggaaagaattggccaccaaacatgtgccaaagttaaacaagcaagggacacgtgtcattcgtaccattccatggcgtttcctagctgggggtgataacagtggtattgcagaagataccagtaaatacccaaatacaccagagggaaataaagctttagccaaagctattgttgatgaatatgtttataaatacaaccttgatggcttagatgtggatgttgaacatgatagtattccaaaagttgacaaaaaagaagatacagcaggcgtagaacgctctattcaagtgtttgaagaaattgggaaattaattggaccaaaaggtgttgataaatcgcggttatttattatggatagcacctacatggctgataaaaacccattgattgagcgaggagctccttatattaatttattactggtacaggtctatggttcacaaggagagaaaggtggttgggagcctgtttctaatcgacctgaaaaaacaatggaagaacgatggcaaggttatagcaagtatattcgtcctgaacaatacatgattggtttttctttctatgaggaaaatgctcaagaagggaatctttggtatgatattaattctcgcaaggacgaggacaaagcaaatggaattaacactgacataactggaacgcgtgccgaacggtatgcaaggtggcaacctaagacaggtggggttaagggaggtatcttctcctacgctattgaccgagatggtgtagctcatcaacctaaaaaatatgctaaacagaaagagtttaaggacgcaactgataacatcttccactcagattatagtgtctccaaggcattaaagacagttatg

SEQ ID NO: 68 - protein, 802 aa, Streptococcus pyogenes
MGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKDL

SEQ ID NO: 69 - DNA, 2406 bp, Streptococcus pyogenes
atgggaaagacagatcagcaggttggtgctaaattggtacaggaaatccgtgaaggaaaacgcggaccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatgaccgaagacaaacgctatgatgtcattgatcaaaaagacattcctgacccagcattaagagaacaaatcattcaacaagttggacagtataaaggcgatttggaacgttataacaagacattggtgcttacaggagataagattcaaaatcttaaaggactagaaaaattaagcaagttacaaaaattagagttgcgccagctatctaacgttaaagaaattactccagaacttttgccggaaagcatgaaaaaagatgctgagcttgttatggtaggcatgactggtttagaaaaactaaaccttagtggtctaaatcgtcaaactttagacggtatagacgtgaatagtattacgcatttgacatcatttgatatttcacataatagtttggacttgtcggaaaagagtgaagaccgtaaactattaatgactttgatggagcaggtttcaaatcatcaaaaaataacggtgaaaaatacggcttttgaaaatcaaaaaccgaaaggttattatcctcagacgtatgataccaaagaaggtcattatgatgttgataatgcagaacatgatattttaactgattttgtttttggaactgttactaaacgtaatacctttattggagacgaagaagcatttgctatctataaagaaggagctgtcgatggtcgacaatatgtgtctaaagactatacttatgaagcttttcgtaaagactataaaggttacaaggttcatttaactgcttctaacctaggagaaacagttacttctaaggtaactgctactactgatgaaacttacttagtagatgtttctgatggggaaaaagttgttcaccacatgaaactcaatataggatctggtgccatcatgatggaaaatctggcaaaaggggctaaagtgattggtacatctggggactttgagcaagcaaagaagattttcgatggtgaaaagtcagatagattcttcacttggggacaaactaactggatagcttttgatctaggagaaattaatcttgcgaaggaatggcgtttatttaatgcagagacaaatactgaaataaagacagatagtagcttaaacgtggctaaaggacgtcttcagattttaaaagatacaactattgatttagaaaaaatggacataaaaaatcgtaaagagtatctgtcgaatgatgaaaattggactgatgttgctcagatggatgatgcaaaagcgatatttaatagtaaattatccaatgttttatctcggtattggcggttttgtgtagatggtggagctagctcttattaccctcaatataccgaacttcaaatcctcggacaacgtttatcaaatgatgtcgctaatacgctgaaggatctg

SEQ ID NO: 70 - protein, 320 aa, Streptococcus pyogenes
PLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLM

SEQ ID NO: 71 - DNA, 960 bp, Streptococcus pyogenes
ccactatatgctggttattttaggacatggcatgatcgtgcttcaacaggaatagatggtaaacagcaacatccagaaaatactatggctgaggtcccaaaagaagttgatatcttatttgtttttcatgaccatacagcttcagatagtccattttggtctgaattaaaggacagttatgtccataaattacatcaacagggaacggcacttgttcagacaattggtgttaacgaattaaatggacgtacaggtttatctaaagattatcctgatactcctgaggggaacaaagctttagcagcagccattgtcaaggcatttgtaactgatcgtggtgtcgatggactagatattgatattgagcacgaatttacgaacaaaagaacacctgaagaagatgctcgtgctctaaatgtttttaaagagattgcgcagttaataggtaaaaatggtagtgataaatctaaattgctcatcatggacactaccctaagtgttgaaaataatccaatatttaaagggatagcggaagatcttgattatcttcttagacaatattatggttcacaaggtggagaagctgaagtggatactataaactctgattggaaccaatatcagaattatattgatgctagccagttcatgattggattctccttttttgaagaatctgcgtccaaagggaatttatggtttgatgttaacgaatacgaccctaacaatcctgaaaaagggaaagatattgaaggaacacgtgctaaaaaatatgcagagtggcaacctagtacaggtggtttaaaagcaggtatattctcttatgctattgatcgtgatggagtggctcatgttccttcaacatataaaaataggactagtacaaatttacaacggcatgaagtcgataatatctcacatactgactacaccgtatctcgaaaattaaaaacattgatg

SEQ ID NO: 72 - protein, 341 aa, Streptococcus pyogenes
MRKRCYSTSAVVLAAVTLFALSVDRGVIADSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 73 - DNA, 1026 bp, Streptococcus pyogenes
atgagaaaaagatgctattcaacttcagctgtagtattggcagcagtgactttatttgctctatcggtagatcgtggtgttatagcagatagtttttctgctaatcaagagattagatattcggaagtaacaccttatcatgttacttccgtttggaccaaaggagttactcctccagcaaaattcactcaaggcgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaaaaaattgaagcatatctaaaaaaacacccagataaacaaaaaatcatgtttggtgatcaagaattattggatgtaagaaaagttattaataccaaaggtgaccaaacaaatagcgagctttttaattatttccgagataaagctttccccggtttgtcagcacgccgaattggagttatgcctgatcttgttttagatatgtttatcaatggttattacttaaatgtttataagacacagactactgatgtcaatagaacctatcaagagaaagatcgccgaggtggtatttttgacgccgtatttacaagaggtgatcaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatattggtgctcaagtactagggttatttacactttcaacagggcaagatagttggaatcagaccaattaa

(19) United States
(12) Patent Application Publication
(10) Pub. No.: US 2024/0150494 A1
(43) Pub. Date: May 9, 2024

(54) GLYCOFORM SPECIFIC NANOBODIES AND METHODS OF USE

(71) Applicant: The Rockefeller University, New York, NY (US)

(72) Inventors: Stylianos Bournazos, New York, NY (US); Jeffrey V. Ravetch, New York, NY (US); Aaron Gupta, New York, NY (US); Kevin Kao, New York, NY (US)

(21) Appl. No.: 18/550,059

(22) PCT Filed: Mar. 10, 2022
(86) PCT No.: PCT/US22/19743

§ 371 Date: Sep. 11, 2023

Related U.S. Application Data
(60) Provisional application No. 63/160,054, filed on Mar. 12, 2021.

(51) Int. Cl. C07K 16/42; A61K 38/47; A61K 38/48; A61K 39/395; A61K 45/06; A61K 47/68; A61P 31/14; C12N 9/24; C12N 9/52; G01N 33/569

(52) U.S. Cl. CPC C07K 16/4216; A61K 38/47; A61K 38/4873; A61K 39/39566; A61K 45/06; A61K 47/6873; A61P 31/14; C12N 9/2402; C12N 9/52; C12Y 302/01096; C12Y 304/2201; G01N 33/56983; C07K 2317/24; C07K 2317/40; C07K 2317/569; C07K 2317/76; C07K 2319/30; G01N 2333/165; G01N 2333/185; G01N 2440/38; G01N 2469/20

(57) ABSTRACT

This disclosure is based, at least in part, on an unexpected discovery that the novel nanobodies and variants thereof are able to specifically bind afucosylated or sialylated IgG Fc glycoforms. Glycosylation of the IgG Fc domain is a major determinant of the strength and specificity of antibody effector functions, modulating the binding interactions of the Fc with the diverse family of Fcγ receptors. These Fc glycan modifications, such as removal of the core fucose residue, are newfound clinical markers for predicting severity of diseases, such as diseases caused by dengue virus (DENV) or SARS-CoV-2. However, it remains challenging to accurately distinguish specific IgG glycoforms without costly and time-intensive methods. The novel glycol-specific nanobodies and variants thereof, as disclosed herein, can be used as rapid clinical diagnostics or prognostics to risk stratify patients with viral and inflammatory diseases.

### CROSS-REFERENCE TO RELATED APPLICATIONS

This application claims priority under 35 U.S.C. § 119(e) to U.S. Provisional Application No. 63/160,054 filed on Mar. 12, 2021, which is hereby incorporated by reference in its entirety.

### STATEMENT REGARDING FEDERALLY SPONSORED RESEARCH

This invention was made with government support under R01AI137276 and U19AI111825 awarded by National Institute of Allergy and Infectious Diseases. The government has certain rights in the invention.

### FIELD OF THE INVENTION

The invention relates to glycoform specific nanobodies and polypeptides and methods of use.

### BACKGROUND OF THE INVENTION

Dengue is the most prevalent arthropod-borne viral disease. Half of the world population lives in areas at risk of infection from dengue virus (DENV), resulting in around 390 million infections each year. Of these, it is estimated that about 300 million remain inapparent and undetected, resulting in insufficient discomfort to disrupt an individual's daily routine. Immune status to DENV is currently considered the greatest risk factor for the requirement of hospitalization after a bite from a DENV-infected mosquito. Depending on the infecting DENV serotype, primary infection commonly leads to inapparent infection or mild disease symptoms, whereas secondary infection can lead to aggravated symptoms requiring hospitalization that can be life-threatening. It is postulated that a mismatch between the infecting serotype and the memory adaptive response leads to an abnormal and exacerbated immune response; however, the details of this mechanism remain to be elucidated. Disease enhancement has been proposed to be due to the presence of pre-existing DENV-reactive IgG antibodies, which at sub-neutralizing levels exacerbate disease by promoting infection of specific leukocyte populations. This phenomenon, termed antibody-dependent enhancement (ADE) of infection, has been extensively studied in in vitro experimental systems and is dependent on the interaction of the IgG Fc domain with Fcγ receptors (FcγRs) expressed on the surface of target cells. Fc-FcγR interactions are thought to promote internalization of IgG-virus immune complexes by FcγR-expressing cells, thereby leading to enhanced frequency of infected cells, increased fusion and/or altered immune responses.

Consistent with a proposed pathogenic role for IgG antibodies, recent epidemiologic studies support that the serum levels of pre-existing anti-DENV antibodies are a key determinant for susceptibility to symptomatic secondary dengue infection. While higher anti-DENV titers confer protection against severe dengue disease, intermediate, sub-neutralizing levels enhance disease through ADE mechanisms. Although immune history and the levels of pre-existing anti-DENV titers represent major risk factors for susceptibility to dengue disease, these factors per se cannot explain why only a small fraction (<5%) of patients with pre-existing anti-DENV IgGs develop severe dengue disease, suggesting the existence of additional host immune factors that contribute to disease susceptibility.

Thus, there remains a strong need for a novel diagnostic and prognostic tool for viral and inflammatory diseases.

### SUMMARY OF THE INVENTION

This disclosure addresses the need mentioned above in a number of aspects. In one aspect, this disclosure provides an isolated nanobody that binds specifically to an IgG Fc glycoform (e.g., IgG1 Fc glycoform). The method comprises three complementarity determining regions (CDR1, CDR2, and CDR3).

In some embodiments, CDR1 comprises an amino acid sequence having at least 90% sequence identity with the amino acid sequence of SEQ ID NO: 1; CDR2 comprises an amino acid sequence having at least 90% sequence identity with the amino acid sequence of SEQ ID NO: 2; and CDR3 comprises an amino acid sequence having at least 90% sequence identity with the amino acid sequence of SEQ ID NO: 3.

In some embodiments, CDR1 comprises an amino acid sequence having at least 90% sequence identity with the amino acid sequence of SEQ ID NO: 5; CDR2 comprises an amino acid sequence having at least 90% sequence identity with the amino acid sequence of SEQ ID NO: 6; and CDR3 comprises an amino acid sequence having at least 90% sequence identity with the amino acid sequence of SEQ ID NO: 7.

In some embodiments, CDR1 comprises an amino acid sequence having at least 90% sequence identity with the amino acid sequence of SEQ ID NO: 9; CDR2 comprises an amino acid sequence having at least 90% sequence identity with the amino acid sequence of SEQ ID NO: 10; and CDR3 comprises an amino acid sequence having at least 90% sequence identity with the amino acid sequence of SEQ ID NO: 11.

In some embodiments, CDR1 comprises an amino acid sequence having at least 90% sequence identity with the amino acid sequence of SEQ ID NO: 13; CDR2 comprises an amino acid sequence having at least 90% sequence identity with the amino acid sequence of SEQ ID NO: 14; and CDR3 comprises an amino acid sequence having at least 90% sequence identity with the amino acid sequence of SEQ ID NO: 15.

In some embodiments, CDR1 comprises an amino acid sequence having at least 90% sequence identity with the amino acid sequence of SEQ ID NO: 17; CDR2 comprises an amino acid sequence having at least 90% sequence identity with the amino acid sequence of SEQ ID NO: 18; and CDR3 comprises an amino acid sequence having at least 90% sequence identity with the amino acid sequence of SEQ ID NO: 19.

In some embodiments, CDR1 comprises an amino acid sequence having at least 90% sequence identity with the amino acid sequence of SEQ ID NO: 21; CDR2 comprises an amino acid sequence having at least 90% sequence identity with the amino acid sequence of SEQ ID NO: 22; and CDR3 comprises an amino acid sequence having at least 90% sequence identity with the amino acid sequence of SEQ ID NO: 23.

In some embodiments, CDR1 comprises an amino acid sequence having at least 90% sequence identity with the amino acid sequence of SEQ ID NO: 25; CDR2 comprises an amino acid sequence having at least 90% sequence identity with the amino acid sequence of SEQ ID NO: 26; and CDR3 comprises an amino acid sequence having at least 90% sequence identity with the amino acid sequence of SEQ ID NO: 27.

In some embodiments, CDR1 comprises an amino acid sequence having at least 90% sequence identity with the amino acid sequence of SEQ ID NO: 29; CDR2 comprises an amino acid sequence having at least 90% sequence identity with the amino acid sequence of SEQ ID NO: 30; and CDR3 comprises an amino acid sequence having at least 90% sequence identity with the amino acid sequence of SEQ ID NO: 31.

In some embodiments, CDR1 comprises an amino acid sequence having at least 90% sequence identity with the amino acid sequence of SEQ ID NO: 33; CDR2 comprises an amino acid sequence having at least 90% sequence identity with the amino acid sequence of SEQ ID NO: 34; and CDR3 comprises an amino acid sequence having at least 90% sequence identity with the amino acid sequence of SEQ ID NO: 35.

In some embodiments, CDR1 comprises the amino acid sequence of SEQ ID NO: 1; CDR2 comprises the amino acid sequence of SEQ ID NO: 2; and CDR3 comprises the amino acid sequence of SEQ ID NO: 3.

In some embodiments, CDR1 comprises the amino acid sequence of SEQ ID NO: 5; CDR2 comprises the amino acid sequence of SEQ ID NO: 6; and CDR3 comprises the amino acid sequence of SEQ ID NO: 7.

In some embodiments, CDR1 comprises the amino acid sequence of SEQ ID NO: 9; CDR2 comprises the amino acid sequence of SEQ ID NO: 10; and CDR3 comprises the amino acid sequence of SEQ ID NO: 11.

In some embodiments, CDR1 comprises the amino acid sequence of SEQ ID NO: 13; CDR2 comprises the amino acid sequence of SEQ ID NO: 14; and CDR3 comprises the amino acid sequence of SEQ ID NO: 15.

In some embodiments, CDR1 comprises the amino acid sequence of SEQ ID NO: 17; CDR2 comprises the amino acid sequence of SEQ ID NO: 18; and CDR3 comprises the amino acid sequence of SEQ ID NO: 19.

In some embodiments, CDR1 comprises the amino acid sequence of SEQ ID NO: 21; CDR2 comprises the amino acid sequence of SEQ ID NO: 22; and CDR3 comprises the amino acid sequence of SEQ ID NO: 23.

In some embodiments, CDR1 comprises the amino acid sequence of SEQ ID NO: 25; CDR2 comprises the amino acid sequence of SEQ ID NO: 26; and CDR3 comprises the amino acid sequence of SEQ ID NO: 27.

In some embodiments, CDR1 comprises the amino acid sequence of SEQ ID NO: 29; CDR2 comprises the amino acid sequence of SEQ ID NO: 30; and CDR3 comprises the amino acid sequence of SEQ ID NO: 31.

In some embodiments, CDR1 comprises the amino acid sequence of SEQ ID NO: 33; CDR2 comprises the amino acid sequence of SEQ ID NO: 34; and CDR3 comprises the amino acid sequence of SEQ ID NO: 35.

In some embodiments, the nanobody or antigen-binding fragment thereof comprises an amino acid sequence having at least 80% sequence identity with the amino acid sequence of SEQ ID NO: 4, 8, 12, 16, 20, 24, 28, 32, or 36. In some embodiments, the nanobody or antigen-binding fragment thereof comprises the amino acid sequence of SEQ ID NO: 4, 8, 12, 16, 20, 24, 28, 32, or 36.

In some embodiments, the nanobody or antigen-binding fragment thereof binds specifically to an IgG1 Fc glycoform. In some embodiments, the nanobody or antigen-binding fragment thereof binds specifically to an afucosylated IgG1 Fc glycoform. In some embodiments, the nanobody or antigen-binding fragment thereof binds specifically to an IgG1 Fc glycoform afucosylated at Asp297 (EU numbering).

In some embodiments, the nanobody or antigen-binding fragment thereof binds specifically to a sialylated IgG1 Fc glycoform.

In some embodiments, the nanobody or antigen-binding fragment thereof competes for binding to the IgG Fc glycoform against a Fcγ receptor IIIA (FcγRIIIA).

In some embodiments, the IgG Fc glycoform is an IgG Fc glycoform of an anti-DENV antibody or an anti-SARS-CoV-2 antibody.

In some embodiments, two or more of the nanobody or antigen-binding fragment thereof are linked to each other directly or via a linker. In some embodiments, the nanobody or antigen-binding fragment thereof oligomerizes as a tetramer.

In some embodiments, the nanobody or antigen-binding fragment thereof is detectably labeled or conjugated to a toxin, a therapeutic agent, a polymer, a receptor, an enzyme, or a receptor ligand. In some embodiments, the polymer is polyethylene glycol (PEG). In some embodiments, the nanobody or antigen-binding fragment thereof is biotinylated.

In some embodiments, the nanobody or antigen-binding fragment thereof is a humanized nanobody.

In another aspect, this disclosure additionally provides an isolated antibody or antigen-binding fragment thereof that binds specifically to an IgG Fc glycoform. The antibody or antigen-binding fragment thereof comprises three heavy chain complementarity determining regions (HCDR1, HCDR2, and HCDR3), wherein: (i) HCDR1 comprises the amino acid sequence of SEQ ID NO: 1; HCDR2 comprises the amino acid sequence of SEQ ID NO: 2; and HCDR3 comprises the amino acid sequence of SEQ ID NO: 3; (ii) HCDR1 comprises the amino acid sequence of SEQ ID NO: 5; HCDR2 comprises the amino acid sequence of SEQ ID NO: 6; and HCDR3 comprises the amino acid sequence of SEQ ID NO: 7; (iii) HCDR1 comprises the amino acid sequence of SEQ ID NO: 9; HCDR2 comprises the amino acid sequence of SEQ ID NO: 10; and HCDR3 comprises the amino acid sequence of SEQ ID NO: 11; (iv) HCDR1 comprises the amino acid sequence of SEQ ID NO: 13; HCDR2 comprises the amino acid sequence of SEQ ID NO: 14; and HCDR3 comprises the amino acid sequence of SEQ ID NO: 15; (v) HCDR1 comprises the amino acid sequence of SEQ ID NO: 17; HCDR2 comprises the amino acid sequence of SEQ ID NO: 18; and HCDR3 comprises the amino acid sequence of SEQ ID NO: 19; (vi) HCDR1 comprises the amino acid sequence of SEQ ID NO: 21; HCDR2 comprises the amino acid sequence of SEQ ID NO: 22; and HCDR3 comprises the amino acid sequence of SEQ ID NO: 23; (vii) HCDR1 comprises the amino acid sequence of SEQ ID NO: 25; HCDR2 comprises the amino acid sequence of SEQ ID NO: 26; and HCDR3 comprises the amino acid sequence of SEQ ID NO: 27; (viii) HCDR1 comprises the amino acid sequence of SEQ ID NO: 29; HCDR2 comprises the amino acid sequence of SEQ ID NO: 30; and HCDR3 comprises the amino acid sequence of SEQ ID NO: 31; or (ix) HCDR1 comprises the amino acid sequence of SEQ ID NO: 33; HCDR2 comprises the amino acid sequence of SEQ ID NO: 34; and HCDR3 comprises the amino acid sequence of SEQ ID NO: 35.

In some embodiments, the antibody or antigen-binding fragment thereof comprises a heavy chain variable region (HCVR) that comprises an amino acid sequence having at least 80% sequence identity with the amino acid sequence of SEQ ID NO: 4, 8, 12, 16, 20, 24, 28, 32, or 36, or comprises the amino acid sequence of SEQ ID NO: 4, 8, 12, 16, 20, 24, 28, 32, or 36.

In another aspect, this disclosure also provides a polypeptide comprising at least one nanobody or antigen-binding fragment thereof, or the antibody or antigen-binding fragment thereof, as described herein. In some embodiments, the polypeptide comprises two or more above-described nanobodies or antigen-binding fragments thereof linked to each other directly or via a linker. In some embodiments, the linker comprises a peptide linker, a nonpeptide linker, or a disulfide bond.

In some embodiments, the polypeptide comprises a first nanobody or antigen-binding fragment thereof and a second nanobody or antigen-binding fragment thereof described above, wherein the first nanobody or antigen-binding fragment thereof and the second nanobody or antigen-binding fragment bind to different epitopes in the IgG Fe glycoform.

In some embodiments, the polypeptide comprises a first nanobody or antigen-binding fragment thereof, a second nanobody or antigen-binding fragment thereof, and a third nanobody or antigen-binding fragment thereof described above, wherein at least two of the first nanobody or antigen-binding fragment thereof, the second nanobody or antigen-binding fragment, and the third nanobody or antigen-binding fragment thereof bind to different epitopes in the IgG Fc glycoform.

In some embodiments, the polypeptide comprises the nanobody or antigen-binding fragment thereof of or the antibody or antigen-binding fragment thereof, linked to an endoglycosidase or proteinase directly or via a linker. In some embodiments, the linker comprises a peptide linker, a nonpeptide linker, or a disulfide bond.

In some embodiments, the endoglycosidase or proteinase comprises EndoS, EndoS2, or IdeS from Streptococcus pyogene. In some embodiments, the endoglycosidase or proteinase comprises an amino acid sequence having at least 80% sequence identity with the amino acid sequence of SEQ ID NO: 64, 66, 68, 70, or 72, or comprises the amino acid sequence of SEQ ID NO: 64, 66, 68, 70, or 72.

In some embodiments, the polypeptide comprises an amino acid sequence having at least 80% sequence identity with the amino acid sequence of SEQ ID NO: 48, 50, 52, 54, 56, 58, 60, or 62, or comprises the amino acid sequence of SEQ ID NO: 48, 50, 52, 54, 56, 58, 60, or 62.

Also within the scope of this disclosure are (a) a nucleic acid molecule comprising a polynucleotide encoding the nanobody or antigen-binding fragment thereof or the polypeptide, as disclosed herein; (b) a vector comprising the nucleic acid molecule described herein; and (c) a cell expressing the nanobody or antigen-binding fragment thereof or the polypeptide, as disclosed herein, or comprising the vector described herein.

In another aspect, this disclosure provides a pharmaceutical composition comprising the nanobody or antigen-binding portion thereof, the polypeptide, the nucleic acid, the vector, or the cell, as disclosed herein, and optionally a pharmaceutically acceptable diluent or carrier.

In another aspect, this disclosure further provides a kit comprising: (a) the nanobody or antigen-binding portion thereof, the polypeptide, the nucleic acid, the vector, the cell, or the pharmaceutical composition, as disclosed herein; and (b) a set of instructions.

In some embodiments, the kit further comprises a detection means. In some embodiments, the detection means comprises a secondary antibody.

In yet another aspect, this disclosure further provides a method of identifying a patient as having an increased risk of a disease or condition. In some embodiments, the method comprises: (i) providing a sample from the patient; (ii) determining a level of an afucosylated IgG Fc glycoform or a sialylated IgG Fc glycoform in the sample using the nanobody or antigen-binding portion thereof or the polypeptide, as disclosed herein; (iii) comparing the determined level of the afucosylated IgG Fc glycoform or the sialylated IgG Fc glycoform to a reference level and determining whether the determined level of the afucosylated IgG Fc glycoform or the sialylated IgG Fc glycoform is elevated as compared to the reference level; and (iv) identifying the patient as having an increased risk of developing the disease or condition if the determined level is elevated as compared to the reference level.

In some embodiments, the step of determining the level of the afucosylated IgG Fc glycoform or the sialylated IgG Fc glycoform comprises determining a level of the afucosylated IgG1 Fc glycoform or the sialylated IgG1 Fc glycoform.

In some embodiments, the step of determining the level of the afucosylated IgG Fc glycoform or the sialylated IgG Fc glycoform comprises determining a level of the IgG1 Fc glycoform afucosylated at Asp297 (EU numbering).

In some embodiments, the afucosylated IgG Fc glycoform or the sialylated IgG Fc glycoform is an afucosylated IgG Fc glycoform or a sialylated IgG Fc glycoform of an anti-DENV antibody or an anti-SARS-CoV-2 antibody.

In some embodiments, the disease or condition is a severe dengue disease caused by a secondary infection by a DENV. In some embodiments, the severe dengue disease is characterized by a severity level of dengue disease selected from dengue fever (DF), dengue hemorrhagic fever (DHF), and dengue shock syndrome (DSS).

In some embodiments, the disease or condition is caused by SARS-CoV-2.

In some embodiments, IgG1 Fc glycoforms comprise at least 3% afucosylated IgG1 Fc glycoforms. In some embodiments, IgG1 Fc glycoforms comprise at least 5% afucosylated IgG1 Fc glycoforms. In some embodiments, IgG1 Fc glycoforms comprise at least 8% afucosylated IgG1 Fc glycoforms.

In yet another aspect, this disclosure additionally provides a method of treating or preventing a virus infection. In some embodiments, the method comprises administering to the patient an effective amount of the nanobody or antigen-binding fragment thereof, the antibody or antigen-binding fragment thereof, the polypeptide, the nucleic acid, the vector, the cell, or the pharmaceutical composition, as described herein. In some embodiments, the virus infection is caused by a dengue virus or a SARS-CoV-2 virus.

In some embodiments, the method comprises identifying the patient as having an increased risk of developing severe dengue disease by the method disclosed herein.

In some embodiments, the method comprises administering to the patient an additional agent or therapy. In some embodiments, the additional agent or therapy comprises an anti-viral agent.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

In the following a brief description of the appended figures will be given. The figures are intended to illustrate the present invention in more detail. However, they are not intended to limit the subject matter of the invention in any way.

FIGS. 1A, 1B, 1C, 1D, 1E, and 1F (collectively “FIG. 1”) show hospitalized cases of dengue disease characterized by elevated levels of afucosylated IgG1 Fc glycoforms. FIG. 1A shows the Fc-associated glycan structure is dynamically regulated during an immune response with the specific addition of saccharide units to the core glycan structure. This process results in the generation of distinct Fc glycoforms that exhibit differential affinity for the various classes of FcγRs. FIGS. 1B and 1C show analysis of the Fc glycan structure of inapparent (day 4-9 post-detection) and hospitalized cases (day 6-10 post-symptom onset) of dengue infection, which revealed that hospitalized cases were characterized by a global elevation in the levels of afucosylated glycoforms of the IgG1 subclass. Such elevation was observed for anti-DENV E protein-specific IgG1 (FIG. 1B) as well as for total IgG1 (FIG. 1C). FIG. 1B: ***p=0.0005; FIG. 1C: ***p=0.0006, ns: not significant. In contrast to IgG1, no differences in the levels of afucosylation were evident for other IgG subclasses (IgG2-4). FIG. 1D shows a correlation of the abundance of afucosylated IgG1 levels of total with antigen (DENV E)-specific IgGs. FIGS. 1E and 1F show that inapparent and hospitalized cases of dengue disease were characterized by comparable levels of bisecting GlnNAc glycoforms whereas galactosylation was increased in hospitalized cases *p=0.03; ***p=0.0003; ns: not significant.

FIGS. 2A, 2B, 2C, 2D, 2E, 2F, and 2G (collectively “FIG. 2”) show that afucosylation is associated with dengue disease severity and correlates with biological features of severe dengue disease. Hospitalized cases of dengue disease comprise a wide spectrum of clinical disease severity, ranging from dengue fever (DF) to dengue hemorrhagic fever (DHF) and dengue shock syndrome (DSS). Disease severity is associated with thrombocytopenia (FIG. 2A; **p=0.0015; ***p=0.0002) and vascular leakage manifested by elevated hematocrit (Hct) (FIG. 2B; **p=0.004; ****p<0.0001). FIG. 2C shows analysis of the Fc glycan structure of total, anti-DENV E, and anti-DENV NS1 IgGs from dengue patients (day 6-10 post-symptom onset) with differential clinical classification of dengue disease, which revealed that severe dengue disease (DSS) is characterized by increased abundance of afucosylated IgG1 glycoforms compared to mild cases (DF). *p=0.016 for total; **p=0.007 for E protein; **p=0.001 for NS-1. FIGS. 2D and 2E show a correlation of the levels of afucosylated IgG1 with platelets and Hct among hospitalized dengue cases. FIG. 2F shows analysis of IgG samples from hospitalized dengue patients obtained at the time of hospital admission (febrile phase; day 2-6 of fever), which revealed that patients that developed DHIF or DSS had significantly higher abundance of afucosylated IgG1 glycoforms at admission compared to DF patients **p=0.006 vs. DHF; **p=0.005 vs. DSS. FIG. 2G shows the ROC analysis confirming that the levels of IgG1 afucosylation at hospital admission are predictive of severe dengue disease.

FIGS. 3A, 3B, 3C, 3D, 3E, and 3F (collectively “FIG. 3”) show that afucosylation, but not pre-existing IgG titers, are associated with susceptibility to severe dengue disease. Analysis of DENV immune status and anti-DENV IgG titers revealed that hospitalized cases (6-10 post-symptom onset) were characterized by higher anti-DENV titers (FIG. 3A) and increased frequency of secondary infections (FIG. 3B) compared to inapparent dengue cases (day 4-9 post-detection). **p=0.006. FIG. 3C shows that when dengue cases were stratified based on DENV immune status, secondary cases were associated with higher anti-DENV IgG titers (***p=0.0005; ****p<0.0001); however, no difference was evident between inapparent and hospitalized dengue cases. In contrast, hospitalized, but not inapparent dengue cases with prior exposure to DENV were characterized by elevated levels of afucosylated anti-DENV E protein IgG1 glycoforms (FIG. 3D).**p=0.0173; ****p<0.0001. FIGS. 3E and 3F show that anti-DENV IgG titers were not associated with dengue clinical disease severity, as no correlation was evident with platelet levels or Hct.

FIGS. 4A, 4B, 4C, 4D, 4E, 4F, and 4G (collectively “FIG. 4”) show that dengue infection specifically modulates IgG Fc fucosylation. FIG. 4A shows the Fc glycan analysis of IgGs obtained from patients with identical clinical disease classification (DF; day 6-10 post-symptom onset), which revealed that secondary DENV infection was associated with elevated IgG afucosylation levels. *p=0.012; ****p=0.0004. FIG. 4B shows that DF patients were analyzed at convalescence (day 23-100 post-symptom onset), and the abundance of IgG1 afucosylated glycoforms was compared to that of the acute phase (day 6-10). Stratification of DF patients based on immune status revealed that primary DF cases exhibited significantly elevated levels of IgG afucosylation at convalescence compared to the acute phase. FIG. 4C shows that matched plasma samples were obtained, and pre- and post-DENV infection and isolated IgGs (total) were analyzed to determine their Fc glycan composition. Patient stratification based on immune status showed that secondary DENV infection was associated with an increase in the levels of afucosylated IgG1 glycoforms. UD: undetermined immune status. To determine whether the observed increase in IgG1 afucosylation is specific for symptomatic secondary DENV infection, Fc glycan composition was analyzed in WNV patients with differential disease severity (FIG. 4D; asymptomatic vs. symptomatic) and WNV immune status (FIG. 4E; primary vs. secondary). In contrast to DENV, no differences in the levels of afucosylated IgG1 glycoforms were observed among WNV patients. Likewise, comparable afucosylated IgG1 levels were evident in serum samples obtained from ZIKV patients at the acute phase of infection or at early convalescence (FIG. 4F). To assess whether pre-existing DENV immunity influences the Fc glycan structure of anti-ZIKV IgG responses, the levels of afucosylated IgG1 glycoforms of anti-ZIKV E protein and anti-ZIKV NS-1 IgGs were determined in ZIKV patients with differential DENV immune history (FIG. 4G). DENV immune status had no impact on the Fc glycosylation of anti-ZIKV IgGs; ns: not significant.

FIGS. 5A, 5B, 5C, 5D, 5E, 5F, 5G, 5H, and 5I (collectively “FIG. 5”) show the abundance of Fc glycoforms in inapparent and hospitalized dengue patients with differential disease severity. FIG. 5A shows that IgG samples obtained from inapparent (day 4-9 post-detection) and hospitalized dengue patients (day 2-6 post-symptom onset) were assessed for the abundance of IgG1 afucosylation. Hospitalized cases were characterized by significantly elevated levels of afucosylated IgG1 glycoforms. *p=0.035. FIGS. 5B-5I show the analysis of the Fc glycan structure of total, and anti-DENV E protein IgGs from dengue patients with differential clinical classification (DF: dengue fever; DHF: dengue hemorrhagic fever; DSS: dengue shock syndrome) of dengue disease revealed no difference in the abundance of afucosylated IgG2 (FIG. 5B) or IgG3/4 (FIG. 5C) subclasses. Likewise, no major differences were observed in the abundance of bisecting GlcNAc (FIGS. 5D-5F) and galactosylated (FIGS. 5G-5I) Fc glycoforms among patient groups.

FIGS. 6A, 6B, 6C, 6D, 6E, 6F, 6G, 6H, 6I, 6J, 6K, and 6L (collectively “FIG. 6”) show that secondary dengue infection in hospitalized cases of dengue disease is characterized by a specific increase in the levels of afucosylation of IgG1 antibodies. The abundance of Fc glycoforms in total IgGs purified from plasma samples of dengue patients with inapparent and hospitalized dengue disease, and differential DENV immune history was assessed. FIG. 6A shows that hospitalized dengue cases with prior history of DENV infection were characterized by specific enrichment in the levels of afucosylated IgG1 glycoforms ****p<0.0001. In contrast, no differences were observed in the abundance of afucosylated IgG2 or IgG3/4 subclasses (FIGS. 6B and 6C). FIG. 6D shows that the analysis of Fc glycosylation in severe dengue patients (DHF and DSS) at the acute phase of infection and at convalescence revealed persistently high levels of afucosylated IgG1 glycoforms. To determine whether dengue infection is associated with specific increase in Fc glycoforms, matched plasma samples were obtained from dengue-infected individuals before (pre) and after (post) infection (FIGS. 6E-6L). Analysis of the Fc glycosylation of plasma IgGs revealed no differences in the abundance of IgG2-4 afucosylation (FIGS. 6E and 6F). Likewise, comparable levels of bisGlcNAc and galactosylation were observed pre- and post-infection; ns: not significant (FIGS. 6G-6L).

FIGS. 7A, 7B, 7C, 7D, 7E, 7F, 7G, and 7H (collectively “FIG. 7”) show that plasma samples from WNV-infected patients were analyzed by ELISA to determine cross-reactivity against DENV (serotypes 1-4) (FIG. 7A), YFV (FIG. 7B), and JEV NS-1 (FIG. 7C). FIG. 7D shows a summary of ELISA data at 1:640 plasma dilution. The levels of afucosylated IgG2-4, bisGlcNAc IgG1, and galactosylated IgG1 Fc glycoforms were assessed in serum samples obtained from ZIKV patients at the acute phase of infection or at early convalescence (FIGS. 7E-7H).

FIGS. 8A, 8B, 8C, 8D, 8E, 8F, 8G, 8H, 8I, 8J, 8K, 8L, 8M, and 8N (collectively “FIG. 8”) show whether pre-existing DENV immunity influences the Fc glycan structure of anti-ZIKV IgG responses, the levels of specific Fc glycoforms (FIG. 8A: afucosylated IgG2; FIG. 8B: afucosylated IgG3/4; FIG. 8C: bisGlcNAc IgG1; FIG. 8D: galactosylated IgG1) of anti-ZIKV E protein and anti-ZIKV NS-1 IgGs were determined in ZIKV patients with differential DENV immune history. DENV immune status had no impact on the Fc glycosylation of anti-ZIKV IgGs. To assess the potential of afucosylated bulk serum IgG to mediate competition effects, the in vivo cytotoxic activity of fucosylated and afucosylated IgG1 glycoforms of an anti-platelet mAb (6A6) was assessed in the presence of excess, non-antigen-specific IgG. FcγR humanized mice (n=4 mice/group; one experiment) were injected with an excess (600 μg) of fucosylated or afucosylated anti-Dengue HA IgG1 mAb (FIG. 8E). Then, mice were treated with 10 μg of an anti-platelet mAb (clone 6A6), expressed as either fucosylated (GOF) or afucosylated (GO) IgG1 glycoforms. Assessment of platelet counts at different timepoints following 6A6 mAb administration revealed increased cytotoxic activity for afucosylated 6A6 glycoforms compared to their fucosylated counterparts, and their activity was not influenced by the presence of excess irrelevant fucosylated or afucosylated anti-HA mAb. Results are presented as the mean±SEM (FIGS. 8F-8N). The abundance of different Fc glycoforms of anti-DENV E protein IgGs from three subjects was assessed by mass spectrometry in two independent experiments (x axes) to determine assay reproducibility.

FIGS. 9A, 9B, 9C, 9D, 9E, 9F, and 9G (collectively “FIG. 9”) show generation of IgG glycoform specific nanobodies. FIG. 9A shows a schematic of the N-linked glycan on Asn-297 of the IgG Fc. FIG. 9B shows the results of liquid chromatography electrospray ionization mass-spectrometry (LC-ESI-MS) of the G2 and G2F glycoforms of rituximab. G2-Fc, M=25374 Da; found (m/z) 25376 (deconvolution data), G2F-Fc, M=25521 Da; found (m/z) 25522 (deconvolution data), S2G2F-Fc, M=26105 Da; found (m/z) 26104. FIG. 9C shows the selection strategy for identification of G2 or S2G2F glycoform-specific nanobodies via magnetic selection (MACS) or fluorescence-activated cell sorting (FACS). Library diversity following five rounds of selection was assessed by next generation sequencing. FIG. 9D shows flow cytometry of yeast displaying C11 with fluorescently labeled IgG1 G2 and G2F glycoforms. FIG. 9E shows binding kinetics of the two dominant clones specific for the G2 glycoform of IgG1 Fc, C11 and D3 evaluated by SPR. Blue or yellow traces are raw data, while 1:1 Langmuir global kinetic fits are shown in black. Top concentration used was 1024 nM with 2-fold serial titration until 32 nM.

FIG. 9F shows flow cytometry of yeast displaying H9 with fluorescently labeled IgG1 G2F and S2G2F glycoforms. FIG. 9G shows binding kinetics of the two dominant clones specific for IgG1 Fc S2G2F, C5, and H9. Blue or yellow traces are raw data, while global kinetic fits are shown in black. Top concentration used was 256 nM with 4-fold serial titration until 16 nM.

FIGS. 10A, 10B, 10C, 10D, 10E, 10F, and 10G (collectively “FIG. 10”) show affinity maturation of C11 yields a nanomolar detection reagent. FIG. 10A shows CDR sequences and dissociation constants (KD) for the G2 and G2F glycoforms for five high affinity clones. FIGS. 10B, 10C, 10D, 10E, and 10F show binding kinetics of B7, X0, mC11, tetrameric B7, or tetrameric FcγRIIIA with G2 or G2F glycoforms of rituximab evaluated by SPR. Blue or yellow traces are raw data, while 1:1 Langmuir global kinetic fits are shown in black. Top concentration used was 256 nM with 2-fold serial titration until 8 nM. FIG. 10G shows Luminex assay comparing the specificity and limit of detection of tetrameric B7 with tetrameric FcγRIIIA for detecting the G2 or G2F glycoforms of rituximab. C11: SEQ ID NO: 9 (CDR1); SEQ ID NO: 10 (CDR2); and SEQ ID NO: 11 (CDR3); B7: SEQ ID NO: 1 (CDR1); SEQ ID NO: 2 (CDR2); and SEQ ID NO: 3 (CDR3); E4: SEQ ID NO: 13 (CDR1); SEQ ID NO: 14 (CDR2); and SEQ ID NO: 15 (CDR3); E2: SEQ ID NO: 17 (CDR1); SEQ ID NO: 18 (CDR2); and SEQ ID NO: 19 (CDR3); X0: SEQ ID NO: 21 (CDR1); SEQ ID NO: 22 (CDR2); and SEQ ID NO: 23 (CDR3); mC11: SEQ ID NO: 5 (CDR1); SEQ ID NO: 6 (CDR2); and SEQ ID NO: 7 (CDR3).

FIGS. 11A, 11B, 11C, 11D, and 11E (collectively “FIG. 11”) show that B7 occupies an overlapping epitope to FcγRIIIA and can block Fc-FcγR interactions. FIG. 11A shows the crystal structure of the B7-IgG1 G2 Fc complex. IgG Fc shown in gray, its glycan at Asn297 in blue, and the B7 nanobody in purple. FIG. 11B shows superimposition of the afucosylated IgG1-FcγRIIIA complex (PDB 3SGK, green) with the B7-IgG1 G2 Fc complex (purple and gray). FIG. 11C shows epitope mapping by SPR shows mutually exclusive binding of B7 and FcγRIIIA to afucosylated IgG1. FIGS. 11D and 11E show enzyme-linked immunoassay (ELISA) evaluating nanobody inhibition of FcγRI or FcγRIIIA binding to afucosylated antibodies or immune complexes, respectively.

FIGS. 12A, 12B, 12C, 12D, and 12E (collectively “FIG. 12”) show that B7 tetramers allow for high-throughput measurement of Fc glycan composition in patient samples. FIGS. 12A and 12B show Luminex assay quantifying afucosylated IgG1 levels in purified IgG or patient serum. FIG. 12C shows correlation of afucosylated IgG1 levels detected in purified IgG versus patient serum. FIG. 12D shows levels of afucosylated IgG1 in dengue patients with variable disease severity. ROC analysis for the predictive value of afucosylated IgG1 levels at hospital admission for progression to severe dengue infection. Pearson correlation analysis for FIGS. 12A-C; One-way ANOVA/Bonferroni post-hoc for FIG. 12D.

FIGS. 13A and 13B (collectively “FIG. 13”) show specificity of sialylated IgG1 Fc-specific nanobodies. Sandwich ELISA demonstrates specific nanobody capture of Rituximab S2G2F by clones H9 and C5.

FIGS. 14A and 14B (collectively “FIG. 14”) show that clone B7 does not bind aglycosylated IgG. Binding kinetics of B7 with anti-NP clone 3B62 IgG1 G2 and its aglycosylated 3B62 N297A mutant are shown. Traces are raw data, while 1:1 Langmuir global kinetic fits are shown in black. Top concentration used was 256 nM with 2-fold serial titration until 16 nM.

FIGS. 15A and 15B (collectively “FIG. 15”) show subclass and glycoform specificity of clone B7. FIG. 15A shows sandwich ELISA evaluating subclass and glycoform specificity of clone B7. Subclass specificity is IgG1>IgG2>IgG3>>IgG4. Binding to fucosylated IgGs is minimal. FIG. 15B shows the human IgG detection reagent in FIG. 15A does not have a preference for subclass or glycoform of IgG. FIG. 15C shows that B7 retains binding to all major afucosylated glycoforms present in human serum.

FIGS. 16A and 16B (collectively “FIG. 16”) show immunoprecipitation of IgG from human serum. FIG. 16A shows SDS-PAGE comparing B7 and mC11 immunoprecipitation of IgG from intact (left three lanes) or IgG-depleted human serum (right three lanes). FIG. 16B shows comparison of intact, IgG-depleted, and IgG-depleted serum reconstituted with Rituximab G2.

FIGS. 17A and 17B (collectively “FIG. 17”) show capture of IgG1 by anti-human IgG1 clone MAI-83240. FIG. 17A shows Luminex quantification of capture of purified patient IgG by beads coated with clone MAI-83240. FIG. 17B shows subclass specificity of clone MAI-83240.

FIG. 18 shows ELISA-based quantification of afucosylated IgG levels in patient serum. Sandwich ELISA demonstrates a strong correlation of OD450 with mass spectrometry determined levels of afucosylated IgG. Statistics are determined by Pearson correlation analysis.

FIG. 19 shows that B cell depletion is blocked by afucosylated IgG-specific nanobodies. The number of B cells (CD45+B220+) was measured by flow cytometry before and one day after administration of rituximab with or without X0-Fc^{N297A}.

### DETAILED DESCRIPTION OF THE INVENTION

This disclosure is based, at least in part, on an unexpected discovery that the novel nanobodies and variants thereof are able to specifically bind afucosylated or sialylated IgG Fc glycoforms. Glycosylation of the IgG Fc domain is a major determinant of the strength and specificity of antibody effector functions, modulating the binding interactions of the Fc with the diverse family of Fcγ receptors. These Fc glycan modifications, such as removal of the core fucose residue, are newfound clinical markers for predicting severity of diseases, such as diseases caused by dengue virus (DENV) or SARS-CoV-2. However, it remains challenging to accurately distinguish specific IgG glycoforms without costly and time-intensive methods. The novel glycol-specific nanobodies and variants thereof, as disclosed herein, can be used as rapid clinical diagnostics or prognostics to risk stratify patients with viral and inflammatory diseases.

### Glycoform-Specific Nanobodies and Polypeptides

Glycoform-Specific Nanobodies and Polypeptides

In one aspect, this disclosure provides an isolated nanobody or antigen-binding fragment thereof that binds specifically to an IgG Fc glycoform (e.g., IgG1 Fc glycoform). The nanobody against an IgG Fc glycoform may have the structure: FR1-CDR1-FR2-CDR2-FR3-CDR3-FR4, in which FR1, FR2, FR3, and FR4 refer to framework regions 1, 2, 3, and 4, respectively, and in which CDR1, CDR2, and CDR3 refer to the complementarity determining regions 1, 2, and 3, respectively.

In some embodiments, the nanobody comprises an amino acid sequence having at least 80% (e.g., 80%, 85%, 90%, 91%, 92%, 93%, 94%, 95%, 96%, 97%, 98%, 99%) sequence identity with one of the amino acid sequences set forth in Table 6. In some embodiments, the nanobody comprises an amino acid sequence differing from an amino acid sequence set forth in Table 6 by 1, 2, 3, 4, 5, 6, 7, 8, 9, or 10 amino acids.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 1; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 2; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 3.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 5; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 6; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 7.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 9; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 10; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 11.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 13; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 14; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 15.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 17; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 18; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 19.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 21; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 22; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 23.

In some embodiments, CDR1 comprises an amino acid sequence having at least 80% (e.g., 80%. 85%. 90%. 91%. 92%. 93%. 94%. 95%. 96%. 97%. 98%. 99%) sequence identity with the amino acid sequence of SEQ ID NO: 25; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 26; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 27.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 29; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 30; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 31.

In some embodiments, CDR1 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 33; CDR2 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 34; and CDR3 comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 35.

In some embodiments, CDR1 comprises the amino acid sequence of SEQ ID NO: 1; CDR2 comprises the amino acid sequence of SEQ ID NO: 2; and CDR3 comprises the amino acid sequence of SEQ ID NO: 3.

In some embodiments, CDR1 comprises the amino acid sequence of SEQ ID NO: 5; CDR2 comprises the amino acid sequence of SEQ ID NO: 6; and CDR3 comprises the amino acid sequence of SEQ ID NO: 7.

In some embodiments, CDR1 comprises the amino acid sequence of SEQ ID NO: 9; CDR2 comprises the amino acid sequence of SEQ ID NO: 10; and CDR3 comprises the amino acid sequence of SEQ ID NO: 11.

In some embodiments, CDR1 comprises the amino acid sequence of SEQ ID NO: 13; CDR2 comprises the amino acid sequence of SEQ ID NO: 14; and CDR3 comprises the amino acid sequence of SEQ ID NO: 15.

In some embodiments, CDR1 comprises the amino acid sequence of SEQ ID NO: 17; CDR2 comprises the amino acid sequence of SEQ ID NO: 18; and CDR3 comprises the amino acid sequence of SEQ ID NO: 19.

In some embodiments, CDR1 comprises the amino acid sequence of SEQ ID NO: 21; CDR2 comprises the amino acid sequence of SEQ ID NO: 22; and CDR3 comprises the amino acid sequence of SEQ ID NO: 23.

In some embodiments, CDR1 comprises the amino acid sequence of SEQ ID NO: 25; CDR2 comprises the amino acid sequence of SEQ ID NO: 26; and CDR3 comprises the amino acid sequence of SEQ ID NO: 27.

In some embodiments, CDR1 comprises the amino acid sequence of SEQ ID NO: 29; CDR2 comprises the amino acid sequence of SEQ ID NO: 30; and CDR3 comprises the amino acid sequence of SEQ ID NO: 31.

In some embodiments, CDR1 comprises the amino acid sequence of SEQ ID NO: 33; CDR2 comprises the amino acid sequence of SEQ ID NO: 34; and CDR3 comprises the amino acid sequence of SEQ ID NO: 35.

In some embodiments, the nanobody or antigen-binding fragment thereof comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 4, 8, 12, 16, 20, 24, 28, 32, or 36. In some embodiments, the nanobody or antigen-binding fragment thereof comprises the amino acid sequence of SEQ ID NO: 4, 8, 12, 16, 20, 24, 28, 32, or 36.

In some embodiments, the nanobody or antigen-binding fragment thereof binds specifically to an IgG1 Fc glycoform. In some embodiments, the nanobody or antigen-binding fragment thereof binds specifically to an afucosylated IgG1 Fc glycoform. In some embodiments, the nanobody or antigen-binding fragment thereof binds specifically to an IgG1 Fc glycoform afucosylated at Asp297 (EU numbering).

In some embodiments, the nanobody or antigen-binding fragment thereof binds specifically to a sialylated IgG1 Fe glycoform.

The term “specifically binds,” or the like, means that an antibody (e.g., nanobody) or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” an afucosylated or sialylated IgG1 Fc glycoform, as used in the context of the present disclosure, includes antibodies that bind an afucosylated or sialylated IgG1 Fc glycoform or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about nM, less than about 5 nM, less than about 4 nM, less than about 3 nM, less than about 2 nM, less than about 1 nM or less than about 0.5 nM, as measured in a surface plasmon resonance assay. An isolated antibody (e.g., isolated nanobody) that specifically binds an afucosylated or sialylated IgG1 Fc glycoform may, however, have cross-reactivity to other antigens, such as an afucosylated or sialylated IgG1 Fc glycoform from other (non-human) species.

In some embodiments, the nanobody or antigen-binding fragment thereof competes for binding to the IgG Fc glycoform against a FcγRIIIA.

In some embodiments, the IgG Fc glycoform is an IgG Fc glycoform of an anti-DENV antibody, an anti-SARS-CoV-2 antibody, or an anti-HIV antibody.

In some embodiments, the nanobody or antigen-binding fragment thereof is a humanized nanobody.

In some embodiments, two or more of the nanobody or antigen-binding fragment thereof are linked to each other directly or via a linker. The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction etc., to provide a site for coupling. Methods for conjugation are well known by persons skilled in the art and are encompassed for use in the present invention. Linker moieties include, but are not limited to, chemical linker moieties, or for example, a peptide linker moiety (a linker sequence).

In some embodiments, the linker can be a peptide linker or a non-peptide linker. Examples of the peptide linker may include, without limitation, [S(G)n]m or [S(G)n]mS, where n may be an integer between 1 and 20, and m may be an integer between 1 and 10.

In some embodiments, the nanobody or antigen-binding fragment thereof may exist as a monomer, dimer, trimer, tetramer, pentamer, and higher-order oligomer. In some embodiments, the nanobody or antigen-binding fragment thereof may oligomerize as a tetramer.

Also within the scope of this disclosure are derivatives of the disclosed nanobodies. Such derivatives can generally be obtained by modification, e.g., by chemical and/or biological (e.g., enzymatical) modification, of the nanobodies of this disclosure and/or of one or more of the amino acid residues that form the nanobodies of this disclosure. For example, such a modification may involve the introduction (e.g., by covalent linking or in another suitable manner) of one or more functional groups, residues or moieties into or onto the nanobody, and of one or more functional groups, residues or moieties that confer one or more desired properties or functionalities to the nanobody.

For example, such modification may comprise the introduction (e.g., by covalent binding or in any other suitable manner) of one or more functional groups that increase the half-life, the solubility, and/or the absorption of the nanobody of this disclosure that reduce the immunogenicity, and/or the toxicity of the nanobody that eliminate or attenuate any undesirable side effects of the nanobody, and/or that confer other advantageous properties to and/or reduce the undesired properties of the nanobodies and/or polypeptides; or any combination of two or more of the foregoing. One of the most widely used techniques for increasing the half-life and/or reducing immunogenicity of pharmaceutical proteins comprises attachment of a suitable pharmacologically acceptable polymer, such as polyethyleneglycol (PEG) or derivatives thereof (such as methoxy polyethylene glycol or mPEG). Any suitable form of pegylation can be used, such as the pegylation used in the art for antibodies and antibody fragments (including but not limited to (single) domain antibodies and ScFv's); reference is made to, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example, from Nektar Therapeutics, USA. For example, a PEG is used with a molecular weight of more than 5000 Dalton, such as more than 10,000 Dalton, and less than 200,000 Dalton, such as less than 100,000 Dalton; for example, in the range of 20,000-80,000 Dalton.

Another modification comprises N-linked or O-linked glycosylation, usually as part of co-translational and/or post-translational modification, depending on the host cell used for expressing the disclosed nanobody or polypeptide.

Yet another modification may comprise the introduction of one or more detectable labels or other signal-generating groups or moieties, depending on the intended use of the labeled nanobody. Suitable labels and techniques for attaching, using and detecting them may include, but are not limited to, fluorescent labels (such as fluorescein, isothiocyanate, rhodamine, phycoerythrin, phycocyanin, allophycocyanin, o-phthaldehyde, and fluorescamine and fluorescent metals such as ¹⁵²Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes (such as ³H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se), metals, metals chelates or metallic cations (for example metallic cations such as ^{99m}Tc, ¹²³I, ¹¹¹In, ¹³¹I, ⁹⁷Ru, ⁶⁷Cu, ⁶⁷Ga, and ⁶⁸Ga or other metals or metallic cations that are particularly suited for use in in vivo, in vitro or in situ diagnosis and imaging, such as (¹⁵⁷Gd, ⁵⁵Mn, ¹⁶²Dy, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, β-galactosidase, ribonuclease, urease, catalase, glucose-VI-phosphate dehydrogenase, glucoamylase and acetylcholine esterase). Other suitable labels may include moieties that can be detected using NMR or ESR spectroscopy.

Such labeled nanobodies and polypeptides of this disclosure may, for example, be used for in vitro, in vivo, or in situ assays (including known immunoassays such as ELISA, RIA, EIA, and other “sandwich assays,” etc.) as well as in vivo diagnostic and imaging purposes, depending on the choice of the specific label.

In some embodiments, a modification may include the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA).

In some embodiments, a modification may include the introduction of a functional group that is one part of a specific binding pair, such as the biotin-streptavidin binding pair. Such a functional group may be used to link the nanobody to another protein, polypeptide, or chemical compound that is bound to the other half of the binding pair, i.e., through formation of the binding pair. For example, a nanobody of this disclosure may be conjugated to biotin and linked to another protein, polypeptide, compound, or carrier conjugated to avidin or streptavidin. For example, such a conjugated nanobody may be used as a reporter, for example, in a diagnostic system where a detectable signal-producing agent is conjugated to avidin or streptavidin. Such binding pairs may, for example, also be used to bind the nanobody to a carrier, including carriers suitable for pharmaceutical purposes. One non-limiting example is the liposomal formulations described by Cao and Suresh, Journal of Drug Targeting, 8, 4, 257 (2000). Such binding pairs may also be used to link a therapeutically active agent to the nanobody.

In some embodiments, the nanobody or antigen-binding fragment thereof is detectably labeled or conjugated to a toxin, a therapeutic agent, a polymer, a receptor, an enzyme, or a receptor ligand. In some embodiments, the polymer is polyethylene glycol (PEG). In some embodiments, the nanobody or antigen-binding fragment thereof is biotinylated.

Variants

In some embodiments, amino acid sequence variants of the nanobodies provided herein are contemplated. For example, it may be desirable to improve the binding affinity and/or other biological properties of the nanobody. Amino acid sequence variants of a nanobody may be prepared by introducing appropriate modifications into the nucleotide sequence encoding the nanobody or by peptide synthesis. Such modifications include, for example, deletions from, and/or insertions into and/or substitutions of residues within the amino acid sequences of the nanobody. Any combination of deletion, insertion, and substitution can be made to arrive at the final construct, provided that the final construct possesses the desired characteristics, e.g., antigen binding.

In some embodiments, nanobody variants having one or more amino acid substitutions are provided. Accordingly, a nanobody of the disclosure can comprise one or more conservative modifications of CDRs or framework regions. A conservative modification or functional equivalent of a peptide, polypeptide, or protein as disclosed herein refers to a polypeptide derivative of the peptide, polypeptide, or protein, e.g., a protein having one or more point mutations, insertions, deletions, truncations, a fusion protein, or a combination thereof. It substantially retains the activity of the parent peptide, polypeptide, or protein (such as those disclosed in this disclosure). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this disclosure are nanobodies having one or more point mutations, insertions, deletions, truncations, a fusion protein, or a combination thereof.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

As used herein, the term “conservative modifications” refers to amino acid modifications that do not significantly affect or alter the binding characteristics of the nanobody containing the amino acid sequence. Such conservative modifications include amino acid substitutions, additions, and deletions. Modifications can be introduced into a nanobody of this disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: (i) amino acids with basic side chains (e.g., lysine, arginine, histidine), (ii) acidic side chains (e.g., aspartic acid, glutamic acid), (iii) uncharged polar side chains (e.g., glycine, asparagine, glutamine, serine, threonine, tyrosine, cysteine, tryptophan), (iv) nonpolar side chains (e.g., alanine, valine, leucine, isoleucine, proline, phenylalanine, methionine), (v) beta-branched side chains (e.g., threonine, valine, isoleucine), and (vi) aromatic side chains (e.g., tyrosine, phenylalanine, tryptophan, histidine).

Non-conservative substitutions will entail exchanging a member of one of these classes for another class.

An exemplary substitutional variant is an affinity matured nanobody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a nanobody with an N-terminal methionyl residue. Other insertional variants of the nanobody molecule include the fusion to the N- or C-terminus of the nanobody to an enzyme (e.g., for ADEPT) or a polypeptide which increases the serum half-life of the nanobody.

In another aspect, this disclosure also provides a polypeptide comprising at least one nanobody or antigen-binding fragment thereof described herein. In some embodiments, the polypeptide comprises two or more above-described nanobodies or antigen-binding fragments thereof linked to each other directly or via a linker (e.g., peptide linker, a nonpeptide linker, a disulfide bond).

In some embodiments, the polypeptide comprises a first nanobody or antigen-binding fragment thereof and a second nanobody or antigen-binding fragment thereof described above, wherein the first nanobody or antigen-binding fragment thereof and the second nanobody or antigen-binding fragment bind to different epitopes in the IgG Fc glycoform.

In some embodiments, the polypeptide comprises a first nanobody or antigen-binding fragment thereof, a second nanobody or antigen-binding fragment thereof, and a third nanobody or antigen-binding fragment thereof described above, wherein at least two of the first nanobody or antigen-binding fragment thereof, the second nanobody or antigen-binding fragment, and the third nanobody or antigen-binding fragment thereof bind to different epitopes in the IgG Fe glycoform.

In some embodiments, the polypeptide comprises a nanobody fused at its N-terminal end, at its C-terminal end, or both at its N-terminal end and at its C-terminal end to at least one further amino acid sequence, i.e., to provide a fusion protein comprising the nanobody and the one or more further amino acid sequences. Such a fusion will also be referred to herein as a “nanobody fusion.” The one or more further amino acid sequence may be any suitable and/or desired amino acid sequences. The further amino acid sequences may or may not change, alter or otherwise influence the properties (e.g., biological properties) of the nanobody, and may or may not add further functionality to the nanobody or the polypeptide of this disclosure. In some embodiments, the further amino acid sequence is such that it confers one or more desired properties or functionalities to the nanobody or the polypeptide disclosed herein. Examples of such amino acid sequences may include all amino acid sequences that are used in peptide fusions based on conventional antibodies and fragments thereof (including but not limited to ScFv's and single domain antibodies) as described in Holliger and Hudson, Nature Biotechnology, 23, 9, 1126-1136 (2005).

In some embodiments, the further amino acid sequence may also provide a second binding site, which binding site may be directed against any desired protein, polypeptide, antigen, antigenic determinant, or epitope (including but not limited to the same protein, polypeptide, antigen, antigenic determinant or epitope against which the nanobody of this disclosure is directed, or a different protein, polypeptide, antigen, antigenic determinant or epitope). For example, the further amino acid sequence may provide a second binding site that is directed against a serum protein (such as, for example, human serum albumin or another serum protein such as IgG) to provide increased half-life in serum. See, e.g., EP 0 368 684, WO 91/01743, WO 01/45746, and WO 04/003019.

In some embodiments, the one or more further amino acid sequences may comprise one or more parts, fragments, or domains of conventional 4-chain antibodies (and in particular human antibodies) and/or of heavy chain antibodies (i.e., nanobodies). For example, a nanobody of this disclosure may be linked to a conventional (preferably human) V_{H} or V_{L} domain or to a natural or synthetic analog of a V_{H} or V_{L} domain, or to another nanobody of this disclosure, optionally via a linker sequence.

In some embodiments, the at least one nanobody may also be linked to one or more CH1, CH2, and/or CH3 domains (e.g., human CH1, CH2, and/or CH3 domains), optionally via a linker sequence. For instance, a nanobody linked to a suitable CH1 domain could be used, e.g., together with suitable light chains, to generate antibody fragments/structures analogous to conventional Fab fragments or F(ab′)2 fragments, but in which one or (in case of an F(ab′)2 fragment) one or both of the conventional. V_{H} domains have been replaced by a nanobody of this disclosure. Also, two nanobodies could be linked to a CH3 domain (optionally via a linker) to provide a construct with an increased half-life in vivo.

In some embodiments, one or more nanobodies of this disclosure may be linked to one or more antibody parts, fragments, or domains that confer one or more effector functions to the polypeptide of this disclosure and/or may confer the ability to bind to one or more Fc receptors. For example, the one or more further amino acid sequences may comprise one or more CH2 and/or CH3 domains of an antibody, such as from a heavy chain antibody (as disclosed herein) and more from a conventional human 4-chain antibody; and/or may form part of Fc region, for example from IgG, from IgE, or from another human Ig. For example, WO 94/04678 describes heavy chain antibodies comprising a Camelid VHH domain or a humanized derivative thereof (i.e., a nanobody), in which the Camelidae CH2 and/or CH3 domain have been replaced by human CH2 and CH3 domains, so as to provide an immunoglobulin that consists of two heavy chains each comprising a nanobody and human CH2 and CH3 domains (but no CH1 domain), which immunoglobulin has the effector function provided by the CH2 and CH3 domains and which immunoglobulin can function without the presence of any light chains. Other amino acid sequences that can be suitably linked to the nanobodies of this disclosure to provide an effector function may be chosen based on the desired effector function(s). See, e.g., WO 04/058820, WO 99/42077, and WO 05/017148.

The further amino acid sequences may also form a signal sequence or leader sequence that directs secretion of the nanobody or the polypeptide of this disclosure from a host cell upon synthesis (for example, to provide a pre-, pro-, or prepro-form of the polypeptide of this disclosure, depending on the host cell used to express the polypeptide of this disclosure).

In some embodiments, a polypeptide of this disclosure can comprise the amino acid sequence of a nanobody, which is fused at its N-terminal end, at its C-terminal end, or both at its N-terminal end and at its C-terminal end with at least one further amino acid sequence. In some embodiments, the further amino acid sequence may include at least one further nanobody to provide a polypeptide that comprises at least two, such as three, four, or five, nanobodies, in which the nanobodies may optionally be linked via one or more linker sequences.

Polypeptides of this disclosure comprising two or more nanobodies will also be referred to herein as “multivalent” polypeptides. For example, a “bivalent” polypeptide comprises two nanobodies, optionally linked via a linker sequence, whereas a “trivalent” polypeptide comprises three nanobodies, optionally linked via two linker sequences; etc. In a multivalent polypeptide, the two or more nanobodies may be the same or different. For example, the two or more nanobodies in a multivalent polypeptide of this disclosure may be directed against the same antigen, i.e., against the same parts or epitopes of the antigen or against two or more different parts or epitopes of the antigen; and/or may be directed against the different antigens; or a combination thereof.

Polypeptides of this disclosure that contain at least two nanobodies, in which at least one nanobody is directed against a first antigen, and at least one nanobody is directed against a second nanobody different from the first antigen, will also be referred to as “multispecific” nanobodies. Thus, a “bispecific” nanobody is a nanobody that comprises at least one nanobody directed against a first antigen and at least one further nanobody directed against a second antigen, whereas a “trispecific” nanobody is a nanobody that comprises at least one nanobody directed against a first antigen, at least one further nanobody directed against a second antigen, and at least one further nanobody directed against a third antigen, etc.

Accordingly, a bispecific polypeptide is a bivalent polypeptide comprising a first nanobody directed against a first antigen and a second nanobody directed against a second antigen, in which the first and second Nanobody may optionally be linked via a linker sequence (as defined herein); whereas a trispecific polypeptide of this disclosure in its simplest form is a trivalent polypeptide of this disclosure (as defined herein), comprising a first nanobody directed against a first antigen, a second nanobody directed against a second antigen and a third nanobody directed against a third antigen, in which the first, second and third nanobody may optionally be linked via one or more, and in particular one and more in particular two, linker sequences.

However, a multispecific polypeptide of this disclosure may comprise any number of nanobodies directed against two or more different antigens. For multivalent and multispecific polypeptides containing one or more V_{HH} domains and their preparation, reference is also made to Conrath et al., J. Biol. Chem., Vol. 276, 10. 7346-7350, as well as to EP 0 822 985.

In some embodiments, the one or more nanobodies and the one or more polypeptides may be directly linked to each other (see, e.g., in WO 99/23221) and/or may be linked to each other via one or more suitable spacers or linkers, or any combination thereof. Suitable spacers or linkers for use in multivalent and multispecific polypeptides may be any linker or spacer used in the art to link amino acid sequences. In some embodiments, the linker or spacer is suitable for use in constructing proteins or polypeptides that are intended for pharmaceutical use.

Examples of spacers include the spacers and linkers that are used in the art to link antibody fragments or antibody domains. These include the linkers that are used in the art to construct diabodies or ScFv fragments. In this respect, however, it should be noted that, whereas in diabodies and in ScFv fragments, the linker sequence used should have a length, a degree of flexibility, and other properties that allow the pertinent V_{H} and V_{L} domains to come together to form the complete antigen-binding site, there is no particular limitation on the length or the flexibility of the linker used in the polypeptide of this disclosure, since each nanobody by itself forms a complete antigen-binding site.

Other suitable linkers generally comprise organic compounds or polymers, in particular those suitable for use in proteins for pharmaceutical use. For instance, polyethyleneglycol moieties have been used to link antibody domains. See, e.g., WO 04/081026.

In some embodiments, when two or more linkers are used in the polypeptides of this disclosure, these linkers may be the same or different. Based on the disclosure herein, the skilled person will be able to determine the optimal linkers for use in a specific polypeptide of this disclosure, optionally after some limited routine experiments.

Linkers for use in multivalent and multispecific polypeptides may include glycine-serine linkers, for example, of the type (glyₓser_{y})_{z}, such as (for example (gly₄ser)₃ or (gly₃ser₂)₃, as described in WO 99/42077, hinge-like regions such as the hinge regions of naturally occurring heavy chain antibodies or similar sequences. For other suitable linkers, reference is also made to the general background art cited above.

Given the specificity of the disclosed nanobody clones for IgG glycoforms, fusions of the nanobody with known endoglycosidases or proteinases are also contemplated. Such nanobody-endoglycosidase/proteinase fusions can be used as a therapeutic avenue for clearing pathogenic IgG. Examples of endoglycosidases or proteinases that can be used in this context include EndoS/EndoS2 and IdeS from Streptococcus pyogenes, with EndoS/EndoS2 having the capacity to hydrolyze the N-linked glycan on IgG and IdeS being able to efficiently degrade IgG. In some embodiments, nanobodies B7 or mC11 can be fused to EndoS, EndoS2, or IdeS or catalytic domain thereof. These fusion proteins can clear pathogenic IgG in the context of viral infections, for example, a dengue virus and SARS-CoV-2 infection, as well as other diseases that are driven by afucosylated IgG.

In some embodiments, the polypeptide comprises the nanobody or antigen-binding fragment thereof of or the antibody or antigen-binding fragment thereof, linked to an endoglycosidase or proteinase directly or via a linker. In some embodiments, the linker comprises a peptide linker, a nonpeptide linker, or a disulfide bond.

In some embodiments, the endoglycosidase or proteinase comprises EndoS, EndoS2, or IdeS from Streptococcus pyogene. In some embodiments, the endoglycosidase or proteinase comprises an amino acid sequence having at least 80% (e.g., 80%, 85%, 90%, 91%, 92%, 93%, 94%, 95%, 96%, 97%, 98%, 99%) sequence identity an the amino acid sequence set forth in Table 8, or comprises an amino acid sequence set forth in Table 8.

In some embodiments, the endoglycosidase or proteinase comprises an amino acid sequence having at least 80% (e.g., 80%, 85%, 90%, 91%, 92%, 93%, 94%, 95%, 96%, 97%, 98%, 99%) sequence identity with the amino acid sequence of SEQ ID NO: 64, 66, 68, 70, or 72, or comprises the amino acid sequence of SEQ ID NO: 64, 66, 68, 70, or 72.

In some embodiments, the polypeptide comprises an amino acid sequence having at least 80% (e.g., 80%, 85%, 90%, 91%, 92%, 93%, 94%, 95%, 96%, 97%, 98%, 99%) sequence identity an the amino acid sequence set forth in Table 8, or comprises an amino acid sequence set forth in Table 8.

In some embodiments, the polypeptide comprises an amino acid sequence having at least 80% (e.g., 80%, 85%, 90%, 91%, 92%, 93%. 94%. 95%. 96%, 97%. 98%, 99%) sequence identity with the amino acid sequence of SEQ ID NO: 48, 50, 52, 54, 56, 58, 60, or 62, or comprises the amino acid sequence of SEQ ID NO: 48, 50, 52, 54, 56, 58, 60, or 62.

In another aspect, this disclosure additionally provides an isolated antibody or antigen-binding fragment thereof that binds specifically to an IgG Fc glycoform. The antibody or antigen-binding fragment thereof comprises three heavy chain complementarity determining regions (HCDR1, HCDR2, and HCDR3), wherein: (i) HCDR1 comprises the amino acid sequence of SEQ ID NO: 1; HCDR2 comprises the amino acid sequence of SEQ ID NO: 2; and HCDR3 comprises the amino acid sequence of SEQ ID NO: 3; (ii) HCDR1 comprises the amino acid sequence of SEQ ID NO: 5; HCDR2 comprises the amino acid sequence of SEQ ID NO: 6; and HCDR3 comprises the amino acid sequence of SEQ ID NO: 7; (iii) HCDR1 comprises the amino acid sequence of SEQ ID NO: 9; HCDR2 comprises the amino acid sequence of SEQ ID NO: 10; and HCDR3 comprises the amino acid sequence of SEQ ID NO: 11; (iv) HCDR1 comprises the amino acid sequence of SEQ ID NO: 13; HCDR2 comprises the amino acid sequence of SEQ ID NO: 14; and HCDR3 comprises the amino acid sequence of SEQ ID NO: 15; (v) HCDR1 comprises the amino acid sequence of SEQ ID NO: 17; HCDR2 comprises the amino acid sequence of SEQ ID NO: 18; and HCDR3 comprises the amino acid sequence of SEQ ID NO: 19; (vi) HCDR1 comprises the amino acid sequence of SEQ ID NO: 21; HCDR2 comprises the amino acid sequence of SEQ ID NO: 22; and HCDR3 comprises the amino acid sequence of SEQ ID NO: 23; (vii) HCDR1 comprises the amino acid sequence of SEQ ID NO: 25; HCDR2 comprises the amino acid sequence of SEQ ID NO: 26; and HCDR3 comprises the amino acid sequence of SEQ ID NO: 27; (viii) HCDR1 comprises the amino acid sequence of SEQ ID NO: 29; HCDR2 comprises the amino acid sequence of SEQ ID NO: 30; and HCDR3 comprises the amino acid sequence of SEQ ID NO: 31; or (ix) HCDR1 comprises the amino acid sequence of SEQ ID NO: 33; HCDR2 comprises the amino acid sequence of SEQ ID NO: 34; and HCDR3 comprises the amino acid sequence of SEQ ID NO: 35.

In some embodiments, the antibody or antigen-binding fragment thereof comprises a heavy chain variable region (HCVR) that comprises an amino acid sequence having at least 80% sequence identity with an amino acid sequence set forth in Table 6, such as SEQ ID NO: 4, 8, 12, 16, 20, 24, 28, 32, or 36, or comprises an amino acid sequence set forth in Table 6, such as SEQ ID NO: 4, 8, 12, 16, 20, 24, 28, 32, or 36.

Nucleic Acids, Vectors, and Cells

A nucleic acid encoding a disclosed nanobody or polypeptide can be in the form of single or double-stranded DNA or RNA. For example, the nucleotide sequences of this disclosure may be genomic DNA, cDNA, or synthetic DNA (such as DNA with a codon usage that has been specifically adapted or optimized for expression in the intended host cell or host organism). In some embodiments, the nucleic acid of this disclosure is in essentially isolated form. The nucleic acid may also be in the form of, be present in and/or be part of a vector, such as, for example, a plasmid, cosmid, or YAC, which again may be in essentially isolated form.

The nucleic acids can be prepared or obtained in a known manner, based on the information on the amino acid sequences for the polypeptides given herein, and/or can be isolated from a suitable natural source. To provide analogs, nucleotide sequences encoding naturally occurring VHH domains can, for example, be subjected to site-directed mutagenesis to provide a nucleic acid encoding the analog. Also, to prepare a nucleic acid or several nucleotide sequences, such as at least one nucleotide sequence encoding a nanobody and, for example, nucleic acids encoding one or more linkers can be linked together in a suitable manner.

The nucleic acid may also be in the form of, be present in and/or be part of a genetic construct (e.g., vector). Such genetic constructs generally comprise at least one nucleic acid of this disclosure that is optionally linked to one or more elements of genetic constructs, such as one or more suitable regulatory elements (e.g., a suitable promoter(s), enhancer(s), terminator(s), etc.).

The nucleic acids and/or the genetic constructs may be used to transform a host cell or host organism. The host or host cell may be any suitable (fungal, prokaryotic or eukaryotic) cell or cell line or any suitable fungal, prokaryotic or eukaryotic organism, such as a bacterial strain, including but not limited to gram-negative strains such as strains of Escherichia coli; and gram-positive strains, such as strains of Bacillus, for example of Bacillus subtilis or of Bacillus brevis; strains of Streptomyces, e.g., Streptomyces lividans; strains of Staphylococcus, e.g., Staphylococcus carnosus; a fungal cell, including but not limited to cells from species of Trichoderma, for example from Trichoderma reesei; or from other filamentous fungi; a yeast cell, including but not limited to cells from species of Saccharomyces, e.g., Saccharomyces cerevisiae; an amphibian cell or cell line, such as Xenopus oocytes; an insect-derived cell or cell line, such as cells/cell lines derived from lepidoptera, including but not limited to Spodoptera SF9 and Sf21 cells or cells/cell lines derived from Drosophila, such as Schneider and Kc cells; a plant or plant cell, for example, in tobacco plants; and/or a mammalian cell or cell line, for example, derived a cell or cell line derived from a human, from the mammals including but not limited to CHO-cells, BHK-cells (e.g., BHK-21 cells), and human cells or cell lines such as HeLa, COS (for example COS-7) and PER. C6 cells; as well as all other hosts or host cells known for the expression and production of antibodies and antibody fragments (including but not limited to (single) domain antibodies and ScFv fragments).

The nanobodies and polypeptides of this disclosure can also be introduced and expressed in one or more cells, tissues, or organs of a multicellular organism. The nucleotide sequences may be introduced into the cells or tissues in any suitable way, e.g., using liposomes, or after they have been inserted into a suitable gene vector (for example, a vector derived from retroviruses, such as adenovirus, or parvoviruses, such as an adeno-associated virus). For expression of the nanobodies in a cell, they may also be expressed as “intrabodies.” See, e.g., WO 94/02610, WO 95/22618, and WO 03/014960.

Compositions and Kits

The nobodies or the polypeptides of this disclosure may be formulated as a pharmaceutical preparation comprising at least one nanobody or polypeptide of this disclosure and at least one pharmaceutically acceptable carrier, diluent, or excipient and/or adjuvant, and optionally one or more further pharmaceutically active polypeptides and/or compounds. For example, the nanobodies and polypeptides of this disclosure can be formulated and administered in any suitable known manner. See, e.g., WO 04/041862, WO 04/041863, WO 04/041865, WO 04/041867, Remington's Pharmaceutical Sciences, 18^{th} Ed., Mack Publishing Company, USA (1990), and Remington, the Science and Practice of Pharmacy, 21th Edition, Lippincott Williams and Wilkins (2005). In some embodiments, the nanobodies and polypeptides of this disclosure may be formulated and administered in any manner known for conventional antibodies and antibody fragments (including ScFv's and diabodies) and other pharmaceutically active proteins.

In another aspect, this disclosure further provides a kit, e.g., for diagnosis or prognosis (e.g., identifying, assisting in identifying, diagnosing, assisting in diagnosing, triaging, or assisting in triaging) of a disease or condition (e.g., dengue) in a subject.

In some embodiments, the kit comprises: (a) the nanobody or antigen-binding portion thereof, the polypeptide, the nucleic acid, the vector, the cell, or the pharmaceutical composition, as disclosed herein; and (b) a set of instructions. In some embodiments, the kit further comprises a detection means. In some embodiments, the detection means comprises a secondary antibody.

In some embodiments, the kit comprises: (i) an agent that binds specifically to an anti-DENV antibody or fragment thereof; and (ii) optionally a set of instructions. In some embodiments, the anti-DENV antibody is an IgG1 antibody (e.g., an afucosylated IgG1 anti-DENV antibody). In some embodiments, an agent is any molecule that binds specifically to an anti-DENV antibody or fragment thereof. An agent “specifically binds” to a target molecule if it binds with greater affinity, avidity, more readily, and/or with greater duration than it binds to other substances. In some embodiments, the agent binds specifically to an afucosylated anti-DENV antibody or fragment thereof. In some embodiments, the agent binds specifically to a fucosylated anti-DENV antibody or fragment thereof. In some embodiments, the agent is reactive to the fucose moiety on the CH2 domain of the anti-DENV antibody.

In another aspect, kits for diagnostic assays for detecting and analyzing afucosylated anti-DENV antibody or antigen-binding portion thereof are provided. Such assays may be carried out by any techniques known and available to the artisan, including but not limited to Western blots, ELISAs, radioimmunoassays, immunohistochemical assays, immunoprecipitations, or other immunochemical assays known in the art. These kits may comprise a nanobody or polypeptide disclosed herein, an afucosylated anti-DENV antibody or antigen-binding portion thereof, and/or detection means for the antibody.

The components of the kit as disclosed herein can be provided in any form, e.g., liquid, dried, or lyophilized form, preferably substantially pure and/or sterile. When the components of the kit are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit. In some embodiments, the kit may further include informational materials. The informational material of the kits is not limited in its form. For example, the informational material can include information about the production of the composition, concentration, date of expiration, batch or production site information, and so forth.

### Methods of Diagnosis and Prognosis

In yet another aspect, this disclosure further provides a method of identifying a patient as having an increased risk of a disease or condition. In some embodiments, the method comprises: (i) providing a sample from the patient; (ii) determining a level of an afucosylated IgG Fc glycoform or a sialylated IgG Fc glycoform in the sample using the nanobody or antigen-binding portion thereof or the polypeptide, as disclosed herein; (iii) comparing the determined level of the afucosylated IgG Fc glycoform or the sialylated IgG Fc glycoform to a reference level and determining whether the determined level of the afucosylated IgG Fc glycoform or the sialylated IgG Fc glycoform is elevated as compared to the reference level; and (iv) identifying the patient as having an increased risk of developing the disease or condition if the determined level is elevated as compared to the reference level.

In some embodiments, the step of determining the level of the afucosylated IgG Fc glycoform or the sialylated IgG Fc glycoform comprises determining a level of the afucosylated IgG1 Fc glycoform or the sialylated IgG1 Fc glycoform.

In some embodiments, the step of determining the level of the afucosylated IgG Fc glycoform or the sialylated IgG Fc glycoform comprises determining a level of the IgG1 Fc glycoform afucosylated at Asp297 (EU numbering).

In some embodiments, the afucosylated IgG Fc glycoform or the sialylated IgG Fc glycoform is an afucosylated IgG Fc glycoform or a sialylated IgG Fc glycoform of an anti-DENV antibody, an anti-SARS-CoV-2 antibody, or an anti-HIV antibody.

In some embodiments, the disease or condition is a severe dengue disease caused by a secondary infection by a DENV. In some embodiments, the severe dengue disease is characterized by a severity level of dengue disease selected from dengue fever (DF), dengue hemorrhagic fever (DHF), and dengue shock syndrome (DSS).

In some embodiments, the disease or condition is caused by SARS-CoV-2 or HIV.

In some embodiments, IgG1 Fc glycoforms comprise at least 1% (e.g., 1%, 2%, 3%, 4%, 5%, 6%, 7%, 8%, 9%10%, 11%, 12%, 13%, 14%, 15%, 16%, 17%, 18%, 19%, 20%) afucosylated IgG1 Fc glycoforms. In some embodiments, IgG1 Fc glycoforms comprise at least 3% afucosylated IgG1 Fc glycoforms. In some embodiments, IgG1 Fc glycoforms comprise at least 5% afucosylated IgG1 Fe glycoforms. In some embodiments, IgG1 Fe glycoforms comprise at least 8% afucosylated IgG1 Fe glycoforms. In some embodiments, IgG1 Fe glycoforms comprise at least 10% afucosylated IgG1 Fe glycoforms. In some embodiments, IgG1 Fe glycoforms comprise at least 12% afucosylated IgG1 Fe glycoforms. In some embodiments, IgG1 Fe glycoforms comprise at least 15% afucosylated IgG1 Fe glycoforms.

A level of afucosylation of an afucosylated or sialylated antibody (e.g., anti-DENV antibody or antigen-binding portion thereof) may be determined using the disclosed nanobody or polypeptide and one or more standard quantitative assays generally known in the art, including those described in WO 2007/055916 and US Application No. 20080286819, the contents which are incorporated by reference. Such assays may include, but are not limited to, competition or sandwich ELISA, a radioimmunoassay, a dot blot assay, a fluorescence polarization assay, a scintillation proximity assay, a homogeneous time-resolved fluorescence assay, a resonant mirror biosensor analysis, and a surface plasmon resonance analysis. For example, in the competition or sandwich ELISA, the radioimmunoassay, or the dot blot assay, the afucosylated anti-DENV antibody or antigen-binding portion thereof can be determined by coupling the assay with a statistical analysis method, such as, for example, Scatchard analysis. Scatchard analysis is widely known and accepted in the art and is described in, for example, Munson et al., Anal Biochem, 107:220 (1980), the contents of which are incorporated herein by reference.

The term “% afucosylation” refers to the level of afucosylation in the Fc region of an antibody (e.g., IgG antibodies). The % afucosylation can be measured by mass spectrometry (MS) and presented as the percentage of afucosylated glycan species (species without the fucose on one Fe domain (minus 1) and on both Fe domains (minus 2) combined) over the entire population of the antibody glycoforms. For example, % afucosylation can be calculated as the percentage of the combined area under the minus one fucose peak and minus two fucose peak over the total area of all glycan species analyzed with a nanobody or polypeptide disclosed herein.

The degree of sialylation refers to the degree of sialylation when the amount of sialic acid N-acetylneuraminic acid (NeuNc or NeuNAc) on the protein/antibody molecule. “Sialylation” refers to the type and distribution of sialic acid residues on polysaccharides and oligosaccharides, for example, N-glycans, O-glycans, and glycolipids.

As used herein, a reference level of afucosylated or sialylated IgG1 Fe glycoforms refers, in some embodiments, to a level of afucosylated or sialylated IgG1 Fe glycoforms in a sample obtained from one or more individuals who do not suffer from dengue infection or disease or suffer from inapparent dengue. The level may be measured on an individual-by-individual basis or on an aggregate basis such as an average. A reference level can also be determined by analysis of a population of individuals who have dengue infection but are not experiencing an acute phase of the disease. A reference sample may be used to obtain such a reference level. A reference sample may be obtained from one or more individuals who do not suffer from dengue infection or disease or suffer from inapparent dengue. A reference sample can also be obtained from a population of individuals who have dengue infection but are not experiencing an acute phase of the disease. In some embodiments, a reference level of a respective sample is from the same individual for whom a diagnosis is sought or whose condition is being monitored, but is obtained at a different time. In certain embodiments, a reference level or sample can refer to a level or sample obtained from the same patient at an earlier time, e.g., weeks, months, or years earlier.

As used herein, the determined level of the afucosylated or or sialylated IgG1 Fc glycoforms is elevated as compared to the reference level refers to a positive change in value from the reference level.

“Biological samples,” as used herein, refer to samples taken or derived from a subject. Examples of biological samples include tissue samples or fluid samples (e.g., blood, plasma, serum, urine, saliva, tears, and other bodily fluids). In some embodiments, the methods described herein comprise obtaining or providing a biological sample. In some embodiments, the biological sample is blood or plasma. In some embodiments, the biological sample is blood. In some embodiments, the biological sample is plasma. In some embodiments, the biological sample is collected at one time point. In some embodiments, the biological sample is collected at less than about 72 hours (e.g., within 24, 48, or 72 hours) after disease onset in the subject (>38 degrees Celsius). In some embodiments, the biological sample is collected at more than one time point (e.g., at less than about 72 hours after disease onset, at about 4-7 days after disease onset, and at about 3-4 weeks after disease onset). In some embodiments, a biological sample is one or more (e.g., 2, 3, 4, 5, or more) biological samples. In some embodiments, an assay is performed on a single sample (or samples from a single time point). However, assays can be performed on samples from two or more time points.

A subject is preferably a human. A subject may be an adult or a child. A subject may be a patient. A subject may present with one or more symptoms, e.g., dengue virus-associated symptoms. Dengue virus-associated symptoms include, but are not limited to, headache, muscle and joint pain, nausea, and rashes. A subject may already be known or suspected of having a dengue virus infection. Determining if a subject has dengue virus infection can be accomplished by methods well-known in the art, e.g., viral titer or serology (see, e.g., Dengue hemorrhagic fever: diagnosis, treatment, prevention, and control. Geneva: World Health Organization, 1997). In some embodiments, a subject may have been previously tested for the presence of dengue virus infection, e.g., by viral titer or serology assay. In some embodiments, a subject has or is suspected of having a dengue virus infection. In some embodiments, a subject is suspected of having a dengue virus infection. In some embodiments, a subject has a dengue virus infection.

In one aspect, this disclosure provides a method of identifying a patient as having an increased risk of developing a severe dengue disease, comprising: (a) providing a biological sample from the patient; (b) determining a level of afucosylated IgG1 Fc glycoforms in the biological sample; (c) comparing the determined level of the afucosylated IgG1 Fc glycoforms to a reference level and determining whether the determined level of the afucosylated IgG1 Fc glycoforms is elevated as compared to the reference level; and (d) determining that the patient has an increased risk of developing severe dengue disease if the determined level of the afucosylated IgG1 Fc glycoforms is elevated as compared to the reference level. In some embodiments, the IgG1 antibody is an anti-DENV antibody.

In some embodiments, severe dengue disease is caused by a secondary infection by a DENV. In some embodiments, the severe dengue disease is characterized by a severity level of dengue disease selected from dengue fever (DF), dengue hemorrhagic fever (DHF), and dengue shock syndrome (DSS).

As used herein, the terms “dengue” and “dengue fever (DF)” are used interchangeably. Dengue fever (DF) and dengue hemorrhagic fever (DHF) are acute febrile diseases found in the tropics, with a geographical spread similar to malaria. Caused by one of four closely related virus serotypes of the genus Flavivirus, family Flaviviridae, each serotype is sufficiently different that there is no cross-protection, and epidemics caused by multiple serotypes (hyperendemicity) can occur. Dengue is transmitted to humans by the mosquito Aedes aegypti (rarely Ae des albopictus).

In some embodiments, It may be desirable to distinguish between subjects with primary infection or secondary infection. Accordingly, in some embodiments, a subject has or is suspected of having a primary dengue virus infection. In some embodiments, a subject has or is suspected of having a secondary dengue virus infection (e.g., a subject who has been previously infected with one dengue virus serotype and now has or is suspected of having another infection with a different Dengue virus serotype). In some embodiments, a subject has or is suspected of having a primary or secondary infection. Primary and secondary dengue infections can be distinguished from each other using assays known in the art, e.g., a haemagglutination inhibition (HI) assay, an IgM antibody capture ELISA, or an IgG avidity assay (see, e.g., De Souza V A, Fernandes S, Araujo E S, Tateno A F, Olivera O M. Use of an immunoglobulin G avidity test to discriminate between primary and secondary dengue virus infections. J Clin Microbiol. 2004 April; 42: 1782-1784; Matheus S, Deparis X, Labeau B, Lelarge J, Movran J, Dussart P.

### Methods of Treatment or Prevention of Virus Infection

In yet another aspect, this disclosure additionally provides a method of treating or preventing a virus infection. In some embodiments, the method comprises administering to the patient an effective amount of the nanobody or antigen-binding fragment thereof, the antibody or antigen-binding fragment thereof, the polypeptide, the nucleic acid, the vector, the cell, or the pharmaceutical composition, as described herein. In some embodiments, the virus infection is caused by a dengue virus or a SARS-CoV-2 virus.

In some embodiments, the method comprises identifying the patient as having an increased risk of developing severe dengue disease by the method disclosed herein.

In some embodiments, the method comprises administering to the patient an additional agent or therapy. In some embodiments, the additional agent or therapy comprises an anti-viral agent (e.g., small organic or inorganic molecules, proteins, peptides, peptidomimetics, polysaccharides, nucleic acids, nucleic acid analogs, and derivatives, or peptides).

In some embodiments, the antiviral agent is selected from balapiravir, chloroquine, celgosivir, ivermectin, or Carica folia. In some embodiments, the antiviral agent is a second nanobody or antibody, or fragment thereof, as described herein, that is different from a first nanobody or antibody, or fragment thereof, as described herein. In some embodiments, the antiviral agent is selected from an alpha-glucosidase I inhibitor (e.g., celgosivir), an adenosine nucleoside inhibitor (e.g., NITD008); an RNA-dependent RNA polymerase (RdRp) inhibitor (e.g., NITD107), an inhibitor of host pyrimidine biosynthesis, e.g., host dihydroorotate dehydrogenase (DHODH) (e.g., NITD-982 and brequinar), an inhibitor of viral NS4B protein (e.g., NITD-618), and an iminosugar (e.g., UV-4).

In some embodiments, the method may further comprise administering a vaccine to the subject, e.g., a dengue virus vaccine, a SARS-CoV2 vaccine. In some embodiments, administration of the antibody molecule is parenteral or intravenous.

In some embodiments, the nanobody or antigen-binding fragment thereof, or the antibody or antigen-binding fragment thereof, or the pharmaceutical composition, as disclosed herein, is administered to the patient intratumorally, intravenously, subcutaneously, intraosseously, orally, transdermally, or sublingually.

In some embodiments, the nanobody or antigen-binding fragment thereof, or the antibody or antigen-binding fragment thereof, or the pharmaceutical composition, as disclosed herein, is administered prophylactically or therapeutically.

In some embodiments, the nanobody or antigen-binding fragment thereof, or the antibody or antigen-binding fragment thereof, or the pharmaceutical composition, as disclosed herein, is administered before, after, or concurrently with the additional agent or therapy.

In some embodiments, the method may comprise monitoring a level of afucosylated IgG1 Fc glycoforms in the patient following the treatment. In some embodiments, the method may additionally comprise evaluating the effect of the treatment by performing an assay on a biological sample. In some embodiments, the assay comprises measuring a level of one or more (e.g., 1, 2, 3, 4, 5, 6, 7, 8, 9, 10, 11, 12, 13, 14, 15, 16, 17, 18, 19, 20, 25, 30, 35, 40, 45, 50, 55, 60, 70, 80, 90, or 100) biomarkers (e.g., blood lymphocyte count, presence of anti-DENV IgG, 3L-1b, Il-4, IL-17, FGF-basic, G-CSF, IFN-gamma, RANTES, SAA serum protein, 3-nitrotyrosine protein adduct). Examples of the biomarkers include those described in WO2014081974A1, the content of which is incorporated by reference in its entirety. The level of biomarkers measured may be a nucleic acid, e.g., RNA, level or a protein level or both. Both protein and nucleic acid detection methods are well known in the art (see, e.g., Green and Sambrook. (2012) Molecular Cloning: A Laboratory Manual (Fourth Edition), Current Protocols in Cell Biology. Wiley Online Library. ISBN: 9780471143031, Current Protocols in Molecular Biology. Wiley Online Library. ISBN: 9780471142720, or Walker. Methods in Molecular Biology. Springer Press. ISSN: 1064-3745, which are incorporated herein by reference). Examples of protein assays/detection methods include immunoassays (also referred to herein as immune-based or immuno-based assays, e.g., Western blot and ELISA), Mass spectrometry, and multiplex bead-based assays. Binding partners for protein detection can be designed using methods known in the art and as described herein. Examples of nucleic acid detection methods include Northern blot analysis, quantitative RT-PCR, microarray or probe hybridization, sequencing, and multiplex bead-based assays. Designing nucleic acid binding partners, such as probes, is well known in the art. In some embodiments, the nucleic acid binding partners bind to a part of or an entire nucleic acid sequence of one or more biomarkers.

In some embodiments, the methods may be those known in the art for detecting clinical features as described above (see, e.g., Geneva (1997) World Health Organization. Dengue Hemorrhagic Fever: diagnosis, treatment, prevention, and control, McPhee (2012). Current Medical Diagnosis & Treatment. McGraw-Hill Medical; 52 edition, or Longo et al. (2011) Harrison's Principles of Internal Medicine: Volumes 1 and 2, McGraw-Hill Professional; 18th Edition.).

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “nanobody,” as used herein, is not limited to a specific biological source or to a specific method of preparation. For example, as will be discussed in more detail below, the nanobodies of this disclosure can be obtained (1) by isolating the V_{HH} domain of a naturally occurring heavy chain antibody; (2) by expression of a nucleotide sequence encoding a naturally occurring V_{HH} domain; (3) by “humanization” (as described below) of a naturally occurring V_{HH} domain to or by expression of a nucleic acid encoding a such humanized V_{HH} domain; (4) by “camelization” (as described below) of a naturally occurring V_{H} domain from any animal species, in particular a species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) using synthetic or semi-synthetic techniques for preparing proteins, polypeptides or other amino acid sequences; (7) by preparing a nucleic acid encoding a nanobody using techniques for nucleic acid synthesis, followed by expression of the nucleic acid thus obtained; and/or (8) by any combination of the foregoing.

One class of nanobodies of this disclosure comprises nanobodies with an amino acid sequence that corresponds to the amino acid sequence of a naturally occurring V_{HH} domain, but that has been “humanized,” i.e., by replacing one or more amino acid residues in the amino acid sequence of the naturally occurring V_{HH} sequence by one or more of the amino acid residues that occur at the corresponding position(s) in a V_{H} domain from a conventional 4-chain antibody from a human being (e.g., indicated above). This can be performed in a known manner, for example, on the basis of the further description below and the prior art on humanization referred to herein. Another class of nanobodies of this disclosure comprises nanobodies with an amino acid sequence that corresponds to the amino acid sequence of a naturally occurring VH domain that has been “camelized,” i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a known manner, for example, on the basis of the further description below. Reference is also made to WO 94/04678. Such camelization may occur at amino acid positions which are present at the VH-VL interface and at the so-called Camelidae hallmark residues (see, e.g., WO 94/04678), as also mentioned below.

The term “immunoglobulin sequence,” whether it is used herein to refer to a heavy chain antibody (e.g., nanobody) or to a conventional 4-chain antibody, is used as a general term to include both the full-size antibody, the individual chains thereof, as well as all parts, domains or fragments thereof (including but not limited to antigen-binding domains or fragments such as VHH domains or VH/VL domains, respectively). In addition, the term “sequence” as used herein (for example, in terms like “immunoglobulin sequence,” “antibody sequence,” “variable domain sequence,” “VHH sequence,” or “protein sequence”), should generally be understood to include both the relevant amino acid sequence as well as nucleic acid sequences or nucleotide sequences encoding the same, unless the context requires a more limited interpretation.

The amino acid residues of a nanobody can be numbered according to the general numbering for VH domains given by Kabat et al. (US Public Health Services, NIH Bethesda, Md., Publication No. 91). Alternative methods for numbering the amino acid residues of VH domains, which methods can also be applied in an analogous manner to VHH domains from Camelids and to nanobodies, are the method described by Chothia et al. (Nature 342, 877-883 (1989)), the so-called “AbM definition” and the so-called “contact definition.” However, in the present description, claims, and figures, the numbering according to Kabat as applied to VHH domains by Riechmann and Muyldermans will be followed, unless indicated otherwise.

An “isolated antibody” or “isolated nanobody,” as used herein, is intended to refer to an antibody that is substantially free of other antibodies having different antigenic specificities. An isolated antibody can be substantially free of other cellular material and/or chemicals.

The variable domains present in naturally occurring heavy chain antibodies (or nanobodies) will also be referred to as “VHH domains” in order to distinguish them from the heavy chain variable domains that are present in conventional 4-chain antibodies (which will be referred to hereinbelow as “VH domains”) and from the light chain variable domains that are present in conventional 4-chain antibodies (which will be referred to hereinbelow as “VL domains”).

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is composed of a heavy chain variable region (abbreviated herein as V_{H}) and a heavy chain constant region. The heavy chain constant region is composed of three domains, C_{H}1, C_{H}2, and C_{H}3. Each light chain is composed of a light chain variable region (abbreviated herein as V_{L}) and a light chain constant region. The light chain constant region is composed of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

Accordingly, the terms “antibody” and “antibodies” include full-length antibodies, antigen-binding fragments of full-length antibodies, and molecules comprising antibody CDRs, VH regions or VL regions. Examples of antibodies include monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), scFv-Fcs, camelid antibodies (e.g., llama antibodies), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies disclosed herein refer to polyclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies disclosed herein are IgG antibodies, or a class (e.g., human IgG₁ or IgG₄) or subclass thereof. In a specific embodiment, the antibody is a humanized monoclonal antibody.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993)); (iv) a Fd fragment consisting of the V_{H} and C_{H}I domains; (v) a Fv fragment consisting of the V_{L} and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, et al. (2004). Nat Med. 10(8):871-5.

As used herein, the term “variable region” (variable region of a light chain (VL), variable region of a heavy chain (VH)) denotes each of the pair of light and heavy chains which is involved directly in binding the antibody to the antigen.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.” Antibodies of this disclosure can be of any isotype (e.g., IgA, IgG, IgM, i.e., an α, γ or μ heavy chain). For example, the antibody is of the IgG type. Within the IgG isotype, antibodies may be IgG1, IgG2, IgG3 or IgG4 subclass, for example, IgG1. Antibodies of this disclosure may have a κ or a λ light chain. In some embodiments, the antibody is of IgG1 type and has a κ light chain.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgA1 antibody) and the constant region sequence or Fc is derived from a different source (e.g., a different antibody, such as an IgG, IgA2, IgD, IgE or IgM antibody).

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In some embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “epitope” as used herein refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In some embodiments, epitopes may include determinants that are chemically active surface groupings of molecules such as amino acids, sugar side chains, phosphoryl groups, or sulfonyl groups, and, In some embodiments, may have specific three-dimensional structural characteristics, and/or specific charge characteristics. An epitope typically includes at least 3, 4, 5, 6, 7, 8, 9, 10, 11, 12, 13, 14, or 15 amino acids in a unique spatial conformation. Methods for determining what epitopes are bound by a given antibody (i.e., epitope mapping) are well known in the art and include, for example, immunoblotting and immune-precipitation assays, wherein overlapping or contiguous peptides from a Spike or S protein are tested for reactivity with a given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide; for example, unique subsequences (e.g., as determined by standard sequence comparison and alignment techniques) of the polynucleotides disclosed herein are also encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide; for example, unique subsequences (e.g., as determined by standard sequence comparison and alignment techniques) of the polypeptides disclosed herein are also encompassed by the invention.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

The percent identity between two amino acid sequences can be determined using the algorithm of E. Meyers and W. Miller (Comput. Appl. Biosci., 4:11-17 (1988)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of this disclosure. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

For determination of protein sequence identify, the values included are those defined as ‘Identities” by NCBI and do not account for residues that are not conserved but share similar properties. In some embodiments, the detectable tag can be an affinity tag. The term “affinity tag,” as used herein, relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, and MBP-tag (MBP: maltose-binding protein). Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of the nanobody or polypeptide. The detectable tag and the affinity tag may also be separated by one or more amino acids. In some embodiments, the detectable tag can be conjugated or linked to the variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins that are joined by a peptide bond or via several peptides. The fusion protein may also comprise a peptide linker between the two domains.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “diagnosis” refers to a predictive process in which the presence, absence, severity or course of treatment of a disease, disorder or other medical condition is assessed. For purposes herein, diagnosis also includes predictive processes for determining the outcome resulting from a treatment. Likewise, the term “diagnosing” refers to the determination of whether a sample specimen exhibits one or more characteristics of a condition or disease. The term “diagnosing” includes establishing the presence or absence of, for example, a target antigen or reagent bound targets, or establishing, or otherwise determining one or more characteristics of a condition or disease, including type, grade, stage, or similar conditions. As used herein, the term “diagnosing” can include distinguishing one form of a disease from another. The term “diagnosing” encompasses the initial diagnosis or detection, prognosis, and monitoring of a condition or disease.

The term “prognosis,” and derivations thereof, refers to the determination or prediction of the course of a disease or condition. The course of a disease or condition can be determined, for example, based on life expectancy or quality of life. “Prognosis” includes the determination of the time course of a disease or condition, with or without a treatment or treatments. In the instance where treatment(s) are contemplated, the prognosis includes determining the efficacy of a treatment for a disease or condition.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In this context, a “normal,” “control,” or “reference” subject, patient or population is/are one(s) that exhibit(s) no detectable disease or disorder, respectively.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The terms “prevent,” “preventing,” “prevention,” “prophylactic treatment” and the like refer to reducing the probability of developing a disorder or condition in a subject, who does not have, but is at risk of or susceptible to developing a disorder or condition.

The terms “decrease,” “reduced,” “reduction,” “decrease,” or “inhibit” are all used herein generally to mean a decrease by a statistically significant amount. However, for avoidance of doubt, “reduced,” “reduction,” “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “composition” or “pharmaceutical composition” refers to a mixture of at least one component useful within this disclosure with other components, such as carriers, stabilizers, diluents, dispersing agents, suspending agents, thickening agents, and/or excipients. The pharmaceutical composition facilitates administration of one or more components of this disclosure to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound and are physiologically acceptable to the subject. Supplementary active compounds may also be incorporated into the compositions.

As used herein, the term “risk” refers to a predictive process in which the probability of a particular outcome is assessed.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of this disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, l2%, 1%, 10%, 9%, 8%, 7%, 6%, 5%, 4% 3% 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in the subsequent EXAMPLES.

### Dengue Patient Recruitment, Diagnosis, and Classification

The design and recruitment of patients have been described before (E. Simon-Loriere et al., Sci Transl Med 9, (2017); S. Ly et al., Asymptomatic Dengue Virus Infections, Cambodia, 2012-2013; Emerg Infect Dis 25, 1354-1362 (2019)). Briefly, hospitalized dengue cases were identified from patients presenting with acute dengue-like illness between June and October of 2012 and 2013 at Kampong Cham City Provincial Hospital and two district hospitals in Kampong Cham province. Plasma specimens were tested for the presence of DENV using nested qRT-PCR at the Institut Pasteur du Cambodge, the National Reference Center for arboviral diseases in Cambodia (K. D. Hue et al., J Virol Methods 177, 168-173 (2011)). Patients were diagnosed as acute DENV-infected as following: a positive qRT-PCR or NS1 positive by rapid test (SD Bioline Dengue Duo kits from Standard Diagnostics, Abbott, Chicago, IL, USA) at hospital admission, or seroconversion from DENV-IgM negative to IgM positive during the hospital stay (admittance and discharge sample). Platelet counts and hematocrit were determined by complete blood count at hospital admittance, and patients were classified for severity according to WHO 1997 criteria upon discharge (W. H. Organization, Dengue hemorrhagic fever: diagnosis, treatment, prevention and control (ed. 2nd Edition, 1997)). For the current study, 48 patients were included. Total IgG and anti-DENV IgG characteristics were analyzed at 6-10 days after onset of symptoms, as well as at 2-6 days after onset of symptoms (day of hospital admission) and at convalescence (23-100 days after onset of symptoms) (Table 1). A cluster investigation was initiated, enrolling all family members in the household and people living within a 200-meter radius of the home of the hospitalized dengue cases. Here, individuals were diagnosed as acute DENV-infected by nested qRT-PCR at the time of blood sampling. Individuals were questioned about the history of symptoms 4 days before and were followed up until 10 days after sampling for the occurrence of symptoms (including but not limited to fever, rash, headache, retro-orbital pain). For this study, 23 individuals were included. Individuals were classified as inapparent dengue if they remained asymptomatic (n=19) or developed mild fever (n=4) during the follow up period. Total IgG and anti-DENV IgG characteristics were investigated on day 4-9 after RT-qPCR confirmed infection. In addition, a blood sample was obtained 4 days before qRT-PCR confirmed DENV infection in 11 of the 23 inapparent cases and of 7 additional individuals requiring medical attention (Table 1). In all individuals, immune status to DENV was determined by hemagglutinin-inhibition test to DENV2 and DENV3 and to other flaviviruses circulating in the area, such as Japanese encephalitis of paired acute and convalescent samples (W. H. Organization, Dengue hemorrhagic fever: diagnosis, treatment, prevention and control (ed. 2nd Edition, 1997)). For all individuals, plasma was separated by centrifugation and stored at −80° C. until further analysis. Sample collection was approved by the National Ethics Committee of Health Research of Cambodia, and written informed consent of all participants or legal representatives for participants under 16 years of age was obtained before inclusion in the study.

TABLE 1
Demographics and clinical parameters of DENV-infected patients.

| | | | | | Hospitalized ^{#} | | | | | |
|---|---|---|---|---|---|---|---|---|---|---|
| | Inapparent | | DF | | DHF | | DSS | | Pre-Post | |
| Number | 23 | | 20 | | 16 | | 12 | | 18 | |
| Age at diagnosis | | | | | | | | | | |
| Mean (SD) | 9.8 | (6) | 9.6 | (3) | 9.3 | (3) | 9.8 | (3) | 9.7 | (6) |
| Gender n, (%) | | | | | | | | | | |
| Male | 14 | (60.9) | 9 | (45) | 8 | (50) | 7 | (58.3) | 7 | (38.9) |
| Female | 9 | (39.1) | 11 | (55) | 8 | (50) | 5 | (41.7) | 11 | (61.1) |
| Immune Status* n, (%) | | | | | | | | | | |
| Primary | 11 | (47.8) | 9 | (45) | 0 | (0) | 0 | (0) | 9 | (50) |
| Secondary | 10 | (43.5) | 9 | (45) | 15 | (93.8) | 10 | (83.3) | 7 | (38.9) |
| UD | 2 | (8.7) | 2 | (10) | 1 | (6.3) | 2 | (16.6) | 2 | (11.1) |
| Infecting Serotype\hat{ } n, (%) | | | | | | | | | | |
| DENV1 | 16 | (69.6) | 17 | (85) | 13 | (81.3) | 4 | (33.3) | 11 | (61.1) |
| DENV2 | 5 | (21.7) | 0 | (0) | 2 | (12.5) | 3 | (25) | 4 | (22.2) |
| DENV4 | 2 | (8.7) | 3 | (15) | 0 | (0) | 0 | (0) | 3 | (16.7) |

| | | | | | | | | | |
|---|---|---|---|---|---|---|---|---|---|
| UD | 0 | 0 | (0) | 1 | (6.3) | 5 | (41.7) | 0 | (0) |

| | | | | | |
|---|---|---|---|---|---|
| Viral load\hat{ } (median copies/ml) | 2.4 × 10³ | 1.1 × 10⁶ | 8.3 × 10⁴ | 6.2 × 10⁴ | 4.0 × 10⁴ |

^{#} Categorized according to the WHO 1997 criteria;
*Determined by an HI test on acute and convalescent samples;
\hat{ }Determined by qRT-PCR.
UD: undetermined;
N/A: not applicable

### ZIKV and WNV Patient Cohorts

All plasma samples were de-identified prior to use in the present study. Experiments were performed in compliance with federal laws and institutional guidelines and have been approved by the IRB of the Rockefeller University.

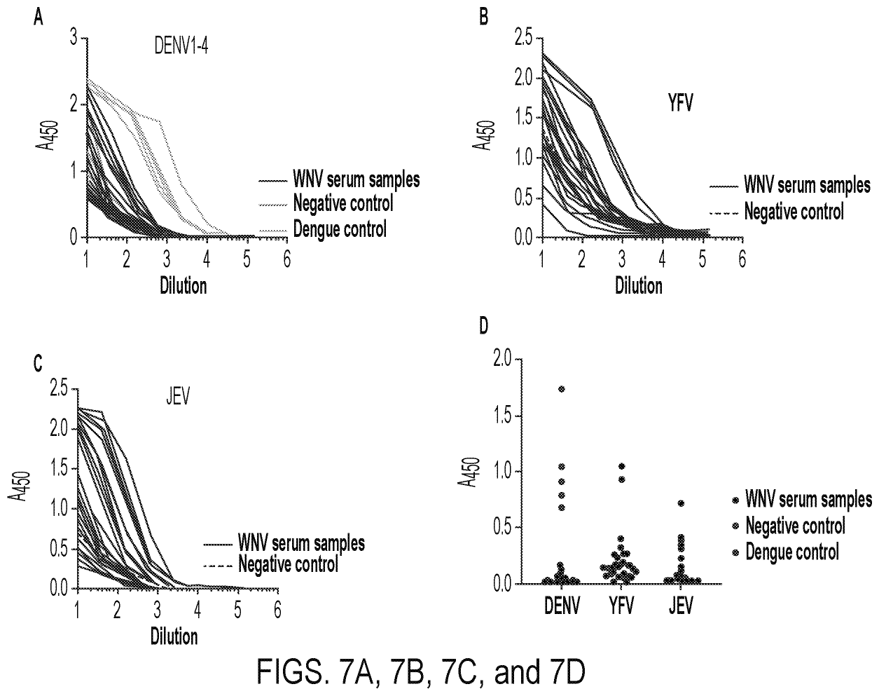

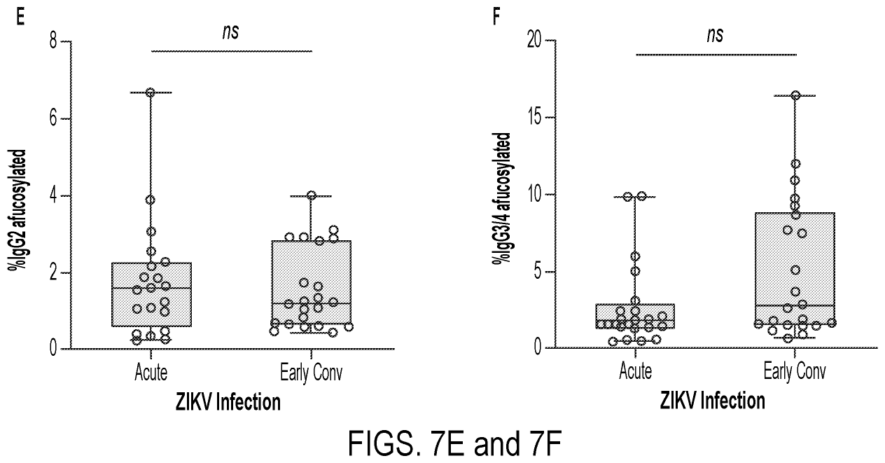

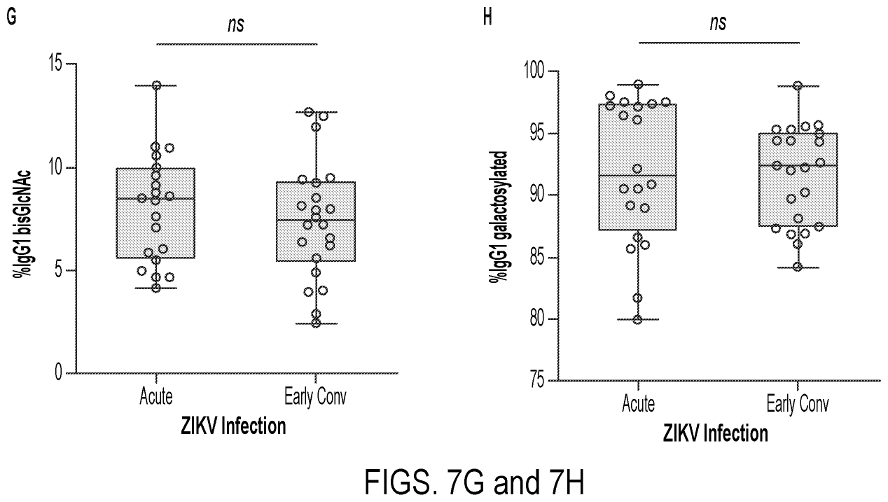

Plasma samples from patients with confirmed WNV infection were obtained from the NHLBI Biologic Specimen and Data Repository Information Coordinating Center (BioLINCC)—WNV study Accession Number HLB01941414a. Details on the design of the clinical study are described in previous publications (H. J. Ramos et al., PLoS Pathog 8, e1003039 (2012).) and at the BIOLINCC portal (biolincc.nhlbi.nih.gov/studies/wnv/). Briefly, all study participants were positive for WNV RNA, and WNV immune status was determined by anti-WNV IgG and IgM ELISA. Anti-WNV IgG and IgM ELISA reagents likely exhibit cross-reactivity against other flaviviruses, and it is possible that subjects classified as secondary WNV cases might actually reflect prior infection with other flaviviruses. Therefore, NV plasma samples were analyzed by ELISA against NS-1 from other flaviviruses (dengue virus (DENV), yellow fever virus (YFV), Japanese encephalitis virus (JEV)) to determine the immune status of the WNV cohort against these flaviviruses (FIGS. 7A-D). Based on clinical questionnaires on patient symptoms, WNV-infected cases were categorized as symptomatic if they exhibited at least one neurological symptom (memory problems, disorientation, confusion, muscle weakness) and/or persistent (lasting >1 w) headache and eye pain. Details on the WNV cohort are presented in Table 2.

TABLE 2
Characteristics of asymptomatic and
symptomatic WNV-infected patients.

| | Asymptomatic | Symptomatic |
|---|---|---|
| Number | 26 | 28 |
| Females (%) | 38.5 | 46.4 |

Immune status (%)

| | | |
|---|---|---|
| Primary | 65.4 | 57.1 |
| Secondary | 34.6 | 42.9 |

Symptoms %

| | | |
|---|---|---|
| Muscle weakness | N/A | 60.7 |
| Confusion | | 21.4 |
| Disorientation | | 10.7 |
| Memory problems | | 7.1 |
| Headache | | 89.3 |
| Eye pain | | 42.9 |
| Fever | | 35.7 |
| Nausea/vomiting | | 39.3 |

Plasma samples from patients with confirmed ZIKV infection were obtained from the BEI Resources, NIAID, NIH. Sample catalog number and information on the ZIKV IgG and IgM reactivity, as well as on DENV immune status (DENV IgG) are presented in Tables 3 and 4.

TABLE 3
Catalogue number (BEI resources, NIAID, NIH) and ZIKV IgM and IgG
reactivity of plasma samples obtained from ZIKV-infected patients
at the acute phase of infection and at early convalescence.

| | | |
|---|---|---|
| Cat Number | ZIKV IgG | ZIKV IgM |

Acute phase of infection

| | | |
|---|---|---|
| NR-50808 | − | − |
| NR-50809 | − | − |
| NR-50810 | − | − |
| NR-50811 | − | − |
| NR-50812 | − | − |
| NR-50813 | − | − |
| NR-50814 | − | − |
| NR-50815 | − | − |
| NR-50816 | − | − |
| NR-50817 | − | − |
| NR-50818 | − | − |
| NR-50819 | − | − |
| NR-50820 | − | − |
| NR-50821 | − | − |
| NR-50822 | − | − |
| NR-50823 | − | − |
| NR-50824 | − | − |
| NR-50825 | − | − |
| NR-50826 | − | − |
| NR-50827 | − | − |

Early Convalescence

| | | |
|---|---|---|
| NR-50828 | + | + |
| NR-50829 | − | + |
| NR-50830 | − | ? |
| NR-50831 | − | ? |
| NR-50832 | − | ? |
| NR-50833 | − | ? |
| NR-50834 | ? | + |
| NR-50835 | + | + |
| NR-50836 | + | ? |
| NR-50837 | − | + |
| NR-50838 | + | + |
| NR-50839 | + | + |
| NR-50840 | + | + |
| NR-50841 | + | + |
| NR-50842 | − | ? |
| NR-50843 | + | + |
| NR-50844 | + | + |
| NR-50845 | + | + |
| NR-50846 | + | + |
| NR-50847 | + | + |
| NR-50850 | + | + |
| NR-50848 | + | + |

+: positive;
−: negative;
?: equivocal

TABLE 4
Catalogue number (BEI Resources, NIAID, NIH), DENV IgG, and
ZIKV IgM and IgG reactivity of plasma samples obtained from
ZIKV-infected patients with differential DENV immune history.

| Cat Number | DENV IgG | ZIKV IgG | ZIKV IgM |
|---|---|---|---|
| NR-50847 | − | + | + |
| NR-50850 | − | + | + |
| NR-50863 | − | + | + |
| NR-50864 | − | + | + |
| NR-50865 | − | + | + |
| NR-50867 | − | + | + |
| NR-50828 | + | + | + |
| NR-50835 | + | + | + |
| NR-50836 | + | + | ? |
| NR-50838 | + | + | + |
| NR-50839 | + | + | + |
| NR-50840 | + | + | + |
| NR-50841 | + | + | + |
| NR-50843 | + | + | + |
| NR-50844 | + | + | + |
| NR-50845 | + | + | + |
| NR-50846 | + | + | + |
| NR-50852 | + | + | + |
| NR-50853 | + | + | + |
| NR-50854 | + | + | + |
| NR-50855 | + | + | + |
| NR-50856 | + | + | + |
| NR-50857 | + | + | + |
| NR-50858 | + | + | + |
| NR-50859 | + | + | + |
| NR-50860 | + | + | + |
| NR-50868 | + | + | + |
| NR-50869 | + | + | + |
| NR-50870 | + | + | + |

+: positive;
−: negative;
?: equivocal

### Foci Reduction Neutralization Test

The FRNT assay was performed using Vero cells (ATCC CCL-81) and DENV-1 and DENV-2 (strain Hawaii and New Guinea, respectively) (H. Auerswald et al., Emerg Microbes Infect 7, 13 (2018)). Foci were stained using polyclonal anti-DENV mouse hyperimmune ascites fluids (IPC, Cambodia) and followed by anti-mouse IgG antibody conjugated to horseradish peroxidase (Biorad). Neutralization was defined as the plasma dilution that induced a 90% reduction in the number of virus-induced foci (foci reduction neutralization test 90%; FRNT90 titer) compared to controls (virus alone and flavivirus-negative control plasma alone) and was calculated via log probit regression analysis (SPSS for Windows v.16.0; SPSS Inc., Chicago, USA). Mean FRNT90 against DENV-1 and DENV-2 is shown.

### IgG Fc Glycan and IgG Subclass Analysis

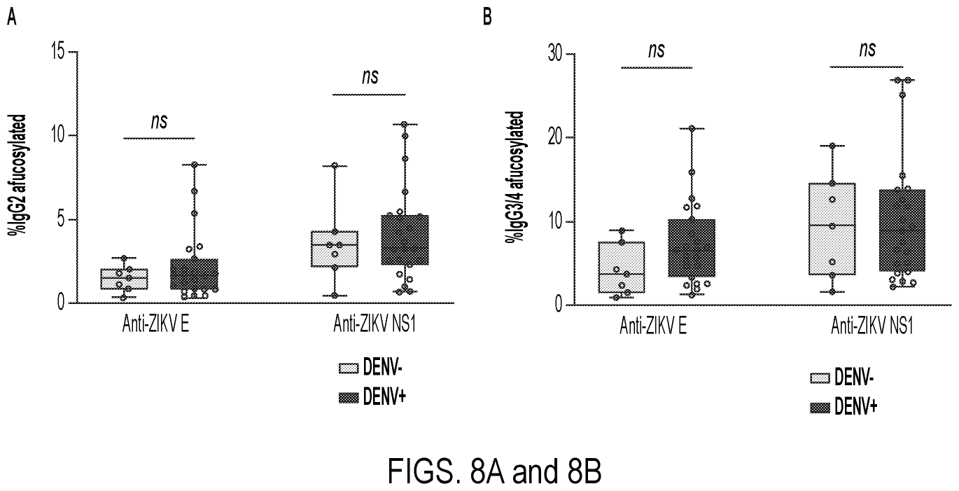

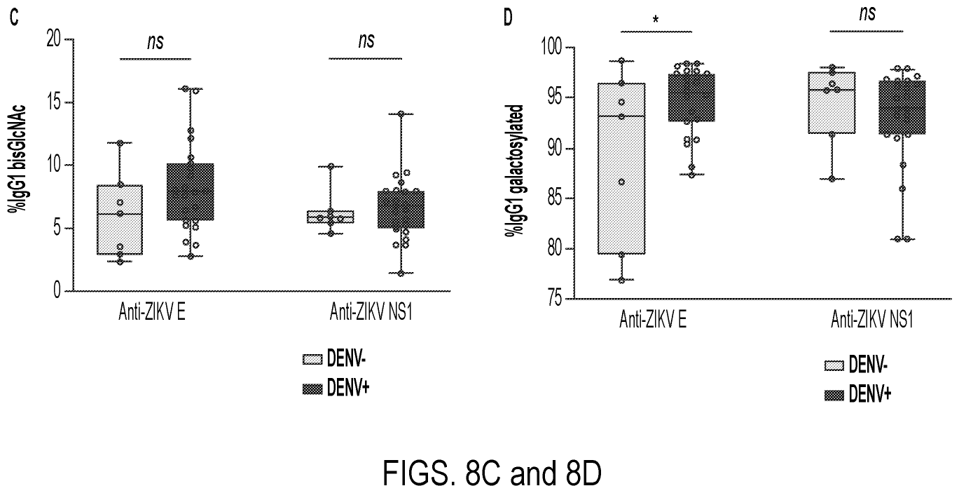

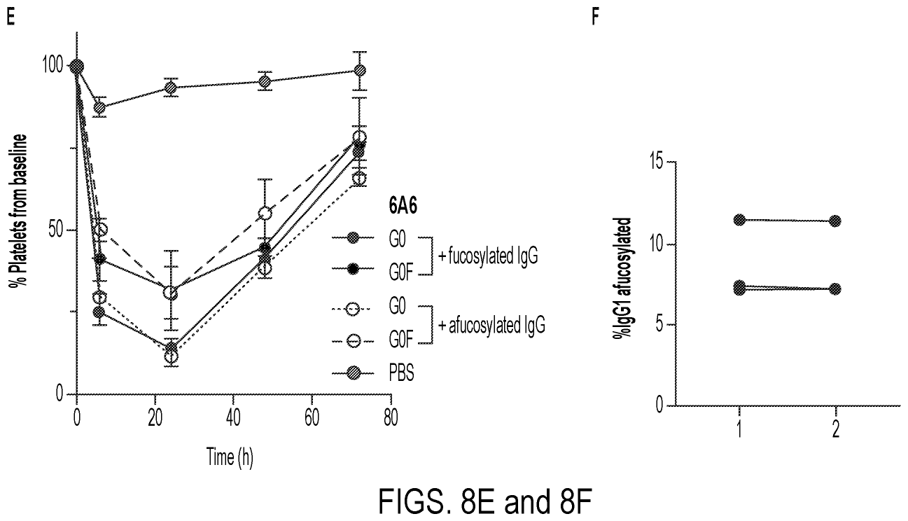

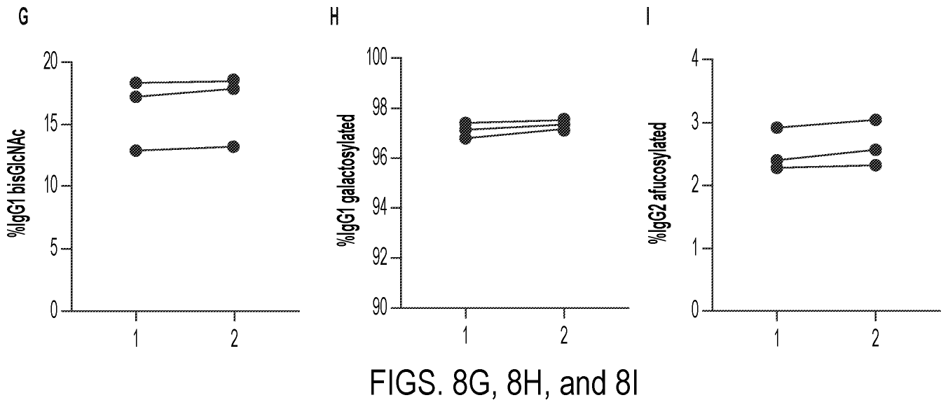

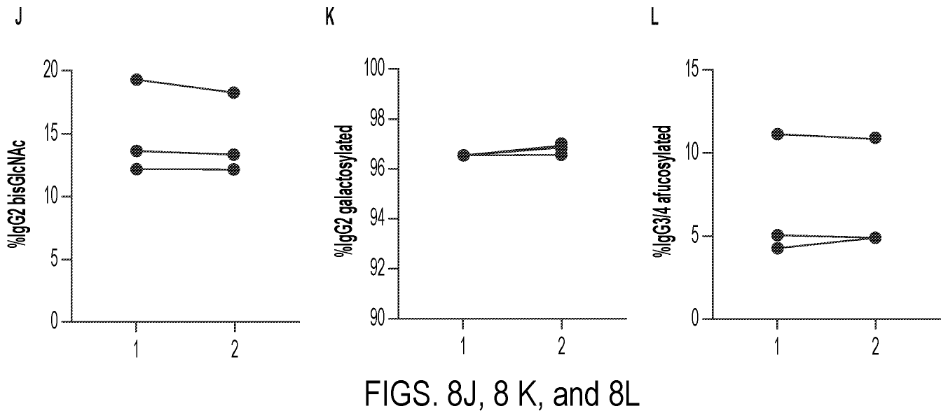

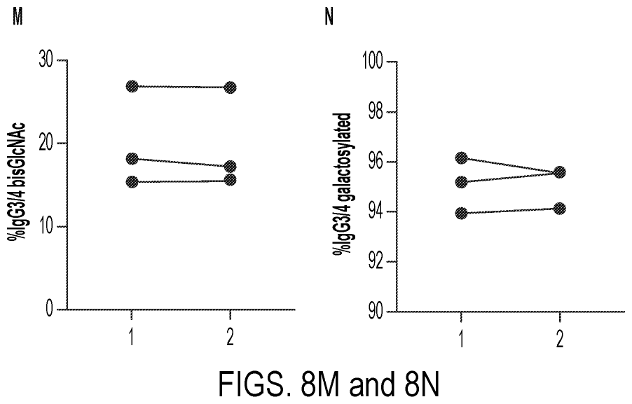

The subclass distribution and Fc glycan composition of total and antigen-specific IgGs were determined by mass spectrometry at the Institute of Biotechnology of the Cornell University, as described previously (18, 31). Briefly, IgGs were purified from plasma or serum samples by protein G purification and dialyzed against PBS. Antigen-specific IgGs were isolated on NHS agarose resin (ThermoFisher) coupled to the relevant protein (DENV1-4 or ZIKV E protein or NS1; Sinobiological or Propecbio). Following tryptic digestion of purified IgGs, nanoLC-MS/MS analysis was performed on tryptic peptides containing the N279 glycan using an UltiMate3000 nanoLC (Dionex) coupled with a hybrid triple quadrupole linear ion trap mass spectrometer, the 4000 Q Trap (SCIEX). Data were acquired using Analyst 1.6.1 software (SCIEX) for precursor ion scan triggered information-dependent acquisition (IDA) analysis for initial discovery-based identification. For quantitative analysis of the glycoforms at the N297 site across the three IgG subclasses (IgG1, IgG2, and IgG3/G4), multiple-reaction monitoring (MRM) analysis for selected target glycopeptides, was applied using the nanoLC-4000 Q Trap platform to the samples after trypsin digestion. The m/z of 4-charged ions for all different glycoforms of the core peptides from three different subclasses as Q1 and the fragment ion at m/z 366.1 as Q3 for each of transition pairs were used for MRM assays. A native IgG tryptic peptide (131-GTLVTVSSASTK-142) (SEQ ID NO: 46) with transition pair of m/z 575.9+2 to mz 780.4 (y8+) was used as a reference peptide for normalization purposes. The IgG subclass distribution was quantitatively determined by nano LC-MRM analysis of tryptic peptides following removal of glycans from purified IgGs with PNGase F. Here, the m/z value of fragment ions for monitoring transition pairs was always larger than that of their precursor ions being multi-charged to enhance the selectivity for unmodified targeted peptides and the reference peptide. All raw MRM data was processed using MultiQuant 2.1.1 (SCIEX). MRM peak areas were automatically integrated and manually inspected. In the event that automatic peak integration by MultiQuant failed, manual integration was performed using the MultiQuant software. Assay reproducibility was determined by assessing the Fc glycan profile from three subjects in two independent experiments. Results are presented in FIGS. 8F-N. Research personnel involved in Fe glycan analysis had no access to clinical information and characteristics of the patient samples.

### ELISA

IgG antibodies were measured with the commercially available Panbio Dengue IgG Indirect ELISA (PanBio; cat no: 01PE30) according to the manufacturer's instructions. Antibody titers were calculated from a dilution range of the kit positive control. Data is reported as Arbitrary Units/ml (AU/ml). To determine flavivirus immune status of the WNV-infected cases, NS-1 from DENV (serotypes 1-4; Biorad), Yellow Fever Virus (Biorad), or Japanese Encephalitis virus (Abcam) was immobilized into high-binding 96-well microtiter plates (Nunc; 5 μg/ml) and after overnight incubation at 4° C., plates were blocked with PBS plus 2% (w/v) BSA and 0.05% (v/v) Tween20 for 2 h. After blocking, plates were incubated for 1 h with serially diluted plasma samples, followed by HRP-conjugated goat anti-human IgG (1 h; 1:5,000; Jackson Immunoresearch; cat no: 109-036-088). Plates were developed using the TMB two-component peroxidase substrate kit (KPL), and reactions were stopped with the addition of 1 M phosphoric acid. Absorbance at 450 nm was immediately recorded using a SpectraMax Plus spectrophotometer (Molecular Devices), and background absorbance from negative control samples was subtracted. Data were collected and analyzed using SoftMax Pro v.7.0.2 software (Molecular Devices).

### Recombinant Antibody Expression and Purification

Recombinant antibodies were generated following previously described protocols (S. Bournazos, et al. Cell 165, 1609-1620 (2016).). Briefly, antibodies were generated by transient transfection of Expi293 cells (ThermoFisher, Cat no: A14635) with heavy- and light-chain expression plasmids. Prior to transfection, plasmid sequences were validated by direct sequencing (Genewiz). Recombinant IgG antibodies were purified from cell-free supernatants by affinity purification using Protein G Sepharose beads (GE Healthcare). Purified proteins were dialyzed in phosphate-buffered saline (PBS) and filter-sterilized (0.22 m), and purity was assessed by sodium dodecyl sulfate/polyacrylamide gel electrophoresis followed by Coomassie blue staining. All antibody preparations were >90% pure, and endotoxin levels were <0.005 endotoxin units/mg, as determined by the Limulus Amebocyte Lysate assay. For the generation of afucosylated Fc domain variants of the anti-HA mAb FI6, CHO cells (ATCC CCL61) were transfected with heavy chain and light chain expression plasmids in the presence of 100 μM 2-fluorofucose peracetate (N. M. Okeley et al., Proc Natl Acad Sci USA 110, 5404-5409 (2013).). The glycoforms of the anti-platelet mAb 6A6 were synthesized by the chemoenzymatic glycan remodeling method (T. Li et al., Proc Natl Acad Sci USA 114, 3485-3490 (2017)).

### In Vivo Platelet Depletion

All in vivo experiments were performed in compliance with federal laws and institutional guidelines and have been approved by the Rockefeller University Institutional Animal Care and Use Committee (Protocol number 20029-H). Mice were bred and maintained at the Comparative Bioscience Center at the Rockefeller University. FcγR humanized mice (FcγRαnull, hFcγRI⁺, FcγRIIaR131⁺, FcγRIIb⁺, FcγRIIIaF158+, and FcγRIIIb⁺) were generated in the C57BL/6 background and have been extensively characterized in previous studies (P. Smith, et al., Proc Natl Acad Sci USA 109, 6181-6186 (2012).). For the platelet depletion model, FcγR humanized mice (males or females, 7-12 weeks old; randomized based on weight, age, and gender) were injected intravenously (i.v.) with 10 μg of recombinant 6A6 human IgG1 mAb glycovariants (GO or GOF). Mice were bled at the indicated time points before and after 6A6 mAb administration, and platelet counts were measured using an automated hematologic analyzer (Heska HT5). Anti-Dengue HA mAbs (clone FI6, expressed as fucosylated or afucosylated) were injected i.p. (600 μg) to mice 6 h prior to 6A6 treatment.

### Statistical Analysis

One- or two-way ANOVA was used to test for differences in the mean values of quantitative variables, and where statistically significant effects were found, posthoc analysis using Bonferroni multiple comparison test was performed. A two-tailed t-test was used to test for differences in datasets with 2 groups (unpaired or paired for matched samples). Pearson correlation analysis was used to assess for correlation between clinical parameters and the abundance of Fc glycoforms. Data were analyzed with Graphpad Prism software (Graphpad), and P values of <0.05 were considered to be statistically significant. No sample size determination analysis was performed.

### Expression and Purification of IgG

Recombinant antibodies were generated using the Expi293 or Expi293 FUT8^{-/-} system (ThermoFisher) using previously described protocols (S. Chakraborty et al., Sci Transl Med, eabm7853 (2022)). Briefly, an equal ratio of heavy and light chain plasmids was complexed with Expifectamine in OptiMEM and added to Expi293 cells in culture at 3×10⁶ cells/ml. Enhancer 1 and Enhancer 2 were added 20 hours after transfection. After 6 days, recombinant IgG antibodies were purified from cell-free supernatants by affinity purification using protein G sepharose beads (GE Healthcare), dialyzed in PBS, filter-sterilized (0.22 m), concentrated with 100 kDa MWCO spin concentrator (Millipore), purified with Superdex 200 Increase 10/300 GL (GE Healthcare), and finally assessed by SDS-PAGE followed by SafeBlue staining (ThermoFisher). All antibody preparations were more than 95% pure, and endotoxin levels were less than 0.05 EU/mg, as measured by the Limulus amebocyte lysate (LAL) assay. Purified IgG was fluorescently labeled with Alexa647-NHS or FITC-NHS (ThermoFisher) at a 15-fold molar excess for 1 hour at room temperature and double-dialyzed into PBS.

### Chemoenzymatic Glycoengineering of IgG

Preparation of (Fucα1,6)GlcNAc-Rituximab with immobilized Endo-S2 WT. Commercial Rituximab (22.0 mg, 100 mg/mL, RefDrug Inc.) was incubated with immobilized (on agarose resin) wild-type Endo-S2 (200:1, wt/wt) at 37° C. with gentle shaking for 6 h, when LC-MS analyses indicated complete cleavage of the N-glycans on the Fc. The resin was centrifuged, and the deglycosylated antibody was purified by protein A chromatography, exchanged to Tris buffer (100 mM, pH 7.2) to yield (Fucα1,6)GlcNAc-Rituximab (21.6 mg, 94%). ESI-MS: calcd for Ides treated Fc of (Fucα1,6)GlcNAc-Rituximab, M=24,108 Da; found (m z), 24,102 Da (deconvolution data).

Preparation of GlcNAc-Rituximab with immobilized Endo-S2 WT and AlfC α-fucosidase in a one-pot manner. To generate GlcNAc-Rituximab, commercial Rituximab (RefDrug Inc., 18.0 mg, 100 mg/mL) was incubated with immobilized wild-type Endo-S2 following the procedure above. After the Fc glycan was completely removed, α-fucosidase AlfC from Lactobacillus casei (50:1, wt/wt) was added to the mixture and incubated at 37° C. for 16 h, when LC-MS analyses indicated complete cleavage of the core fucose on the Fc. The resin was centrifuged down, and the antibody was isolated by purified by protein A chromatography, exchanged to Tris buffer (100 mM, pH 7.2) to yield GlcNAc-Rituximab (15.2 mg, 86%). ESI-MS: caled for Ides treated Fe of GlcNAc-Rituximab, M=23,962 Da; found (m z), 23,956 Da (deconvolution data).

Enzymatic Transglycosylation of (Fucα1,6)GlcNAc-Rituximab or GlcNAc-Rituximab to Generate Rituximab Glycoforms. A solution of (Fucα1,6)GlcNAc-Rituximab (9.0 mg) or GlcNAc-Rituximab (9.0 mg) in a Tris buffer (100 mM, pH 7.2, final antibody concentration 15 mg/mL) and G2-glycan oxazoline (30 eq) was incubated with Endo-S2 D184M mutant (0.05 mg/mL) at 30° C. for 15 min. LC-MS analyses indicated the complete transglycosylation. The mixture was purified by protein A chromatography and exchanged to PBS buffer (100 mM, pH 7.4) to yield G2F-Rituximab (8.1 mg, 88%) or G2-Rituximab (8.3 mg, 90%). ESI-MS: calcd for Ides treated Fc of G2F-Rituximab, M=25,528 Da; found (m z), 25,522 Da (deconvolution data); calcd for Ides treated Fc of G2-Rituximab, M=25,382 Da; found (m z), 25,376 Da (deconvolution data).

### Identification of IgG Fc Glycoform Specific Nanobodies

A previously published yeast surface display library (>5×10⁸ variants) that recapitulates the native llama VHH repertoire (S. Bournazos et al., Science 372, 1102-1105 (2021)) was used. The library displays an HA-tagged nanobody at the terminus of a synthetic stalk sequence, whose expression is controlled by an inducible Gal promoter. In the presence of galactose, 12-18% of the naïve library typically expresses the nanobody protein.

For round 1.5×10⁹ yeast (10× expected diversity) were induced for 48 hours in YEP-galactose tryptophan dropout (−Trp) medium, and washed in staining buffer (20 mM HEPES, pH 7.5, 150 mM sodium chloride, 0.1% (w/v) bovine serum albumin). For negative selection, yeast were resuspended in 5 mL of staining buffer containing 500 nM Rituximab-G2F-Alexa647. Yeast were incubated for 1 hour at 4° C., washed in cold staining buffer, and resuspended in 4.5 mL of staining buffer with 500 uL anti-Alexa647 microbeads (Miltenyi). Yeast were incubated with microbeads for 20 minutes at 4° C., washed in cold staining buffer, and depleted of G2F-binders on a MACS LS column (Miltenyi). For positive selection, yeast were resuspended in 5 mL staining buffer with 500 nM Rituximab-G2-FITC or Rituximab-S2G2F-FITC. Yeast were incubated for 1 hour at 4° C., washed in cold staining buffer, and resuspended in 4.5 mL of staining buffer with 500 uL anti-FITC microbeads. Yeast were incubated with microbeads for 20 minutes at 4° C., washed in cold staining buffer, and G2- or S2G2F-binders were captured on a MACS LS column and recovered in YEP-glucose (−Trp) medium.

For round 2 of selection, 1.5×10⁸ induced yeast, the procedure outlined in round 1 was performed with the fluorophores swapped (i.e., Rituximab-G2F-FITC and Rituximab-G2-Alexa647 or Rituximab-S2G2F-Alexa647). For rounds 3-5, fluorescence-activated cell sorting (FACS) was used in place of MACS. For round 3, 1.5×10⁷ induced yeast were stained with 500 nM Rituximab-G2F-Alexa647 and 250 nM Rituximab-G2-FITC or Rituximab-S2G2F-FITC. FITC⁺Alexa647⁻ clones were sorted into YEP-glucose (−Trp) and expanded. For round 4, 1.5×10⁷ induced yeast were stained with 500 nM Rituximab-G2F-FITC and 250 nM Rituximab-G2-Alexa647 or 250 nM Rituximab-S2G2F-Alexa647. FITC⁻Alexa647⁺ clones were sorted into YEP-glucose (−Trp) and expanded. For round 5, 1.5×10⁷ induced yeast were stained with 500 nM Rituximab-G2F-Alexa647 and 100 nM Rituximab-G2-FITC or 100 nM Rituximab-S2G2F-FITC. FITC⁺Alexa647⁻ clones were sorted into YEP-glucose (−Trp) and expanded.

8×10⁶ yeast were spun down and resuspended in 30 uL 0.2% sodium dodecyl sulfate (v/v) and heated at 94° C. for 4 minutes to lyse yeast. Yeast were spun down at 10000×g, and 1 uL of supernatant was used as a template for a PCR reaction using [primer3, primer4]. Next-generation sequencing of post-round 5 nanobody sequences was performed by a MiSeq Nano (Illumina) with 10% PhiX to yield dominant clones (G2: C11 and D3) and (S2G2F: H9 and C5).

### Expression and Purification of Nanobodies

nanobodies were expressed and purified similarly to previously reported methods (2-4). Nanobody sequences were amplified with [primer 5, primer 6] and cloned into pET26-b(+) expression vector with His tag and AviTag using Gibson Assembly (NEB) and transformed into BL21(DE3) E. coli (NEB). Nanobody multimers were generated using multi-part Gibson Assembly with unique linker regions to preserve correct orientation. Bacteria were grown in terrific broth at 37° C. overnight, and the next day a 1:100 culture was grown until an OD of 0.7-0.9, when 1 mM IPTG was added. After 20-24 hours of shaking at 25° C., E. coli were pelleted and resuspended in SET buffer (200 mM Tris, pH 8.0, 500 mM sucrose, 0.5 mM EDTA, 1× cOmplete protease inhibitor (Sigma)) and rocked for 30 minutes at room temperature, followed by the addition of 2× volume of deionized water and 45 minutes more rocking. NaCl was added to 150 mM, MgCl2 to 2 mM, and imidazole to 20 mM before pelleting cell debris at 17,000×g for 20 minutes. The periplasmic fraction was filtered with a 0.22 um filter and incubated with 4 mL 50% Ni-NTA resin equilibrated in wash buffer (20 mM HEPES, pH 7.5, 150 mM NaCl, 40 mM imidazole) (Qiagen) per liter of initial bacterial culture. Supernatant and resin were rocked for 1 hour at room temperature and then pelleted at 50×g for 1 minute. Resin was washed on a column with 10 volumes of wash buffer before elution with elution buffer (20 mM HEPES, pH 7.5, 150 mM NaCl, 250 mM imidazole). Eluted protein was concentrated with 3 kDa MWCO filters (Amicon) before size-exclusion chromatography (GE Healthcare). Proteins were stable at 4° C. For tetramerization, nanobody monomers were biotinylated in vitro with BirA (Avidity) for 1 hour at room temperature according to the manufacturer's directions, double desalted using Zeba Spin Desalting columns 7K MWCO (ThermoFisher), and purified by size-exclusion chromatography. For in vivo biotinylation, CVB-T7 POL E. coli (Avidity) were used to express nanobodies, and at the time of induction, 50 μM of D-biotin was added to the culture. Streptavidin conjugates were complexed in a 1:4 ratio with biotinylated monomers by adding ¼^{th} volume of conjugate every 10 minutes for a total of 40 minutes.

### Surface Plasmon Resonance

Surface Plasmon Resonance (SPR) was performed on a Biacore T200 machine (Cytiva Life Sciences). In some experiments, purified IgG glycoforms diluted in HBS-EP+ were immobilized on the surface of a Protein A or Protein G CM5 sensor chip at 1000 RU (˜50 nM). Purified nanobodies were flowed over IgG-bound sensor chips at the indicated concentrations at 30 uL/minute for 60 seconds, followed by 600 seconds of dissociation. Sensor chips were regenerated with 10 mM Glycine-HCl pH 1.5.

In other experiments, purified His-tagged nanobodies were immobilized on the Ni²⁺-activated surface of NTA sensor chips at 500 RU (50 nM). Purified IgG was flowed over nanobody-bound sensor chips at the indicated concentrations at 30 uL/minute for 60 seconds, followed by 600 seconds of dissociation. Sensor chips were regenerated with 350 mM EDTA.

For nanobody monomer binding, sensorgrams were fit using a 1:1 Langmuir binding model, and kinetic constants were reported.

### Affinity Maturation of C11

Using degenerate NNK oligos, assembly PCR was used to generate a site saturation mutagenesis library of C11, where one codon in each CDR was mutated at a time, for a total of 0-3 amino acid CDR mutations per nanobody clone. The pooled assembly PCR reaction was amplified so that its ends overlapped with the surface display vector used in the initial rounds of selection. Vector and insert DNA were electroporated into Saccharomyces cerevisiae strain BJ5465 (ATCC 208289) to generate a library of 1.4×10⁷ transformants, which were plated on YEP-glucose (−Trp) agar. Plates were scraped and 1.4×10⁸ induced in YEP-galactose (−Trp) for 48 hours. For round 1, yeast were washed in staining buffer and co-stained with 125 nM Rituximab-G2F-FITC and 2.5 nM Rituximab-G2-Alexa647 (50-fold excess G2F). FITC⁻Alexa647⁺ clones were sorted into YEP-glucose (−Trp), expanded, and induced for round 2. Clones were induced and co-stained with 37.5 nM Rituximab-G2F-Alexa647 and 750 pM Rituximab-G2-FITC (50-fold excess G2F). FITC⁺Alexa647⁻ clones were sorted and plated onto YEP-glucose (−Trp) agar. 288 individual clones were induced in duplicate 96-well plates and stained with 200 pM Rituximab-G2-A647 or 10 nM Rituximab-G2F-A647. Highly selective clones were selected and sequenced for further experiments.

### Nanobody ELISA

For some experiments, half-well 96-well plates were coated with 30 uL of 10 ug/mL mouse anti-IgG1 (ThermoFisher) overnight. Plates were washed with PBST (0.05% Tween-20) 3 times, blocked with 2% BSA in PBS for 1 hour at room temperature, washed, incubated with recombinant IgG, patient purified IgG, or patient serum, washed, incubated with nanobody-streptavidin-HRP conjugates (1:1000, Biolegend), washed, developed with TMB substrate, quenched with 1M phosphoric acid, and read at 450 nm on a spectrophotometer.

For other experiments, half-well 96-well plates were coated with 30 uL of 10 ug/mL nanobody overnight. Plates were washed with PBST (0.05% Tween-20) 3 times, blocked with 2% BSA in PBS for 1 hour at room temperature, washed, incubated with recombinant IgG, patient purified IgG, or patient serum, washed, incubated with anti-human IgG-HRP conjugates (1:5000, JacksonImmunoResearch), washed, developed with TMB substrate, quenched with 1M phosphoric acid, and read at 450 nm on a spectrophotometer.

### Nanobody Luminex

Magplex microspheres (region 45) were conjugated to mouse anti-human IgG1 (ThermoFisher) using xMAP Ab Coupling kit, per manufacturer's instructions, and blocked with 1% BSA in PBS overnight. 50 uL microspheres and 50 uL of diluted recombinant IgG, patient purified IgG, or patient serum were shaken at 500 rpm in a 96-well plate for 1 hour. Microspheres were washed 3 times with 1% BSA in PBS and shaken with nanobody-streptavidin-PE conjugates for 30 minutes. Microspheres were washed, and median fluorescent intensities were calculated using Luminex 200 Instrument System (ThermoFisher).

ELISA-based FcγR binding assay

Recombinant FcγR ectodomains were expressed in Expi-293F and purified with Ni-NTA resin as in previously described protocols (S. Chakraborty et al., Sci Transl Med, eabm7853 (2022)). High-binding 96-well microtiter plates (Nunc) were incubated with 10 ug/mL of recombinant FcγRI or FcγRIIIA(V) overnight at 4° C. Plates were then blocked with PBS plus 2% (w/v) BSA. IgG immune complexes were prepared by incubation of an anti-NP (4-hydroxy-3-nitrophenylacetyl) antibody 3B62 with NP-BSA (27 conjugations) at a 10:1 molar ratio for 1 h at 4° C. Nanobodies were serially diluted 1:3 in PBS, with a starting concentration of 19.2 nM. IgG immune complexes or monomeric 3B62 were brought to a concentration of 20 ug ml⁻¹ or 2 μg/ml respectively and pre-complexed at a 1:1 (v/v) ratio for 1 h at room temperature and then captured on FcγR-coated plates. Following 1 h incubation, bound IgG was detected using a 1:5000 dilution of horseradish peroxidase (HRP)-conjugated goat F(ab′)2 anti-human IgG (H+L) (Jackson Immunoresearch). Plates were developed with TMB (3,3′,5,5′-tetramethylbenzidine) two-component peroxidase substrate kit. Reactions were quenched with 1M phosphoric acid. Absorbance at 450 nm was recorded using a SpectraMax Plus spectrophotometer (Molecular Devices). Background absorbance was subtracted for samples, and % maximum binding was determined against an IgG or immune complex only control.

### IgG Fc Glycan and IgG Subclass Analysis

The subclass distribution and Fc glycan composition of IgGs was determined by mass spectrometry at the Institute of Biotechnology of the Cornell University, as described previously (5, 6). Briefly, IgGs were purified from plasma or serum samples by protein G purification and dialyzed against PBS. Assay reproducibility was determined by assessing the Fc glycan profile from three subjects in two independent experiments. Research personnel involved in Fc glycan analysis had no access to clinical information and characteristics of the patient samples.

### Glycan Array

N-glycan arrays (Z-Biotech) were used according to manufacturer instructions. Briefly, slides were blocked with Glycan Array Blocking Buffer for an hour on a shaker at 85 rpm. After an hour, blocking buffer was removed and 200 uL of B7 (0.5 mg/mL or 0.05 mg/mL) or biotinylated-AAL (10 ug/mL) was added. Slides were incubated for 2 hours, shaking at 200 rpm, and then washed three times with Wash Buffer (50 mM Tris-HCl, 137 mM NaCl, 0.05% Tween 20, pH 7.6). 200 ul of 1 ug/mL of Streptavidin-Cy3 (Vector Labs) was added for 1 hour, shaking at 85 rpm. Slides were washed three times with Wash Buffer, dried, and then scanned with a Typhoon FLA-9500 (GE Healthcare).

### Comigration Studies

Binding of B7 to recombinant human IgG1 G2 Fc was qualitatively assessed by mixing the proteins at a 1:3 molar ratio. The resulting mixture was separated with the use of a Superdex 200 10/300 gel filtration column (GE Healthcare) in HEPES-buffered saline. 1 mL fractions were collected and analyzed on a NuPAGE 4-12% Bis-Tris gel (ThermoFisher).

### Immunoprecipitation

Streptavidin-coated Dynabeads (ThermoFisher) were incubated with 5:1 molar excess biotinylated nanobody for 1 hour at room temperature, then washed,

### B7-IgG1 Fc Crystallography and Structure Determination

B7 was purified from E. coli as described above. The B7-IgG1 Fc complex was created by mixing the two at a molar ratio of 3:1, then subjected to size-exclusion chromatography purification. Purified complex was concentrated to 16 mg/mL and mixed in 200 nL+200 nL drops with Index HT™ screen (Hampton Research HR2-134). Crystals were grown in a sitting-drop format, and subsequent work-up was performed to find ideal crystallization conditions. The final precipitant solution consisted of 0.2 M Sodium citrate tribasic dihydrate, 21% w/v Polyethylene glycol 3,350. Crystals were soaked with 25% glycerol as a cryoprotectant before flash freezing in liquid nitrogen.

Data collection was performed at The Northeastern Collaborative Access Team (NE-CAT) facility at the Advanced Photon Source at Argonne National Laboratory. Diffraction data were collected at an energy of 12.66 keV with a 0.2-s exposure per frame and each frame covered a 0.4° oscillation. The structure of the complex was solved by molecular replacement in Phaser using the solved structures of a nanobody derived from the same synthetic library (PDB 5VNV) and IgG1 Fc portion only from the IgG1 Fc-FcγRIIIB complex (PDB 6EAQ) as search models.

Structural refinement was initially performed in Phenix by rigid body refinement using the Cγ2 and Cγ3 domains of IgG1 Fc as well as the nanobody as independent rigid molecules. Group B-factor refinement with two groups per residue was used in the final stages of refinement. Crystallographic data analysis was performed with xds and phenix.refine, using standard metrics to assess structure quality. Full details of crystallographic statistics are summarized in Table S1.

### Generation of FUT8 Knockout Expi-293F Cell Lines

CRISPR-Cas9 guide RNAs targeting human FUT8 were assembled with Cas9-3NLS nuclease (Synthego) via incubation at 37° C. for 15 min. Cas9/RNP complexes were nucleofected into 2×10⁶ cells using the SF Cell Line 4D-Nucleofector kit according to manufacturer's instructions (Lonza). After a week of culture, indel frequencies were quantified using TIDE software as described previously (S. Chakraborty et al., Nat Immunol, (2020)). The sequence for the single-guide RNA (sgRNA) molecule used is as follows: ACAGCCAAGGGTAAATATGG (SEQ ID NO: 47).

### Patient Samples

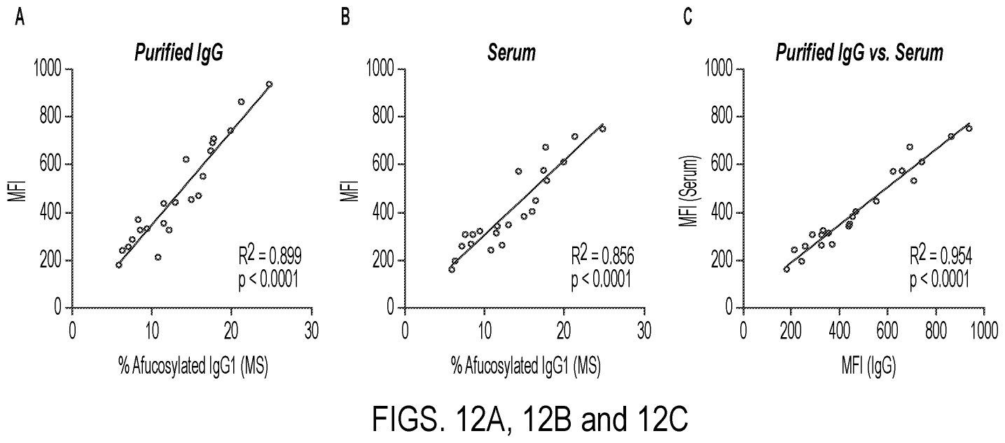

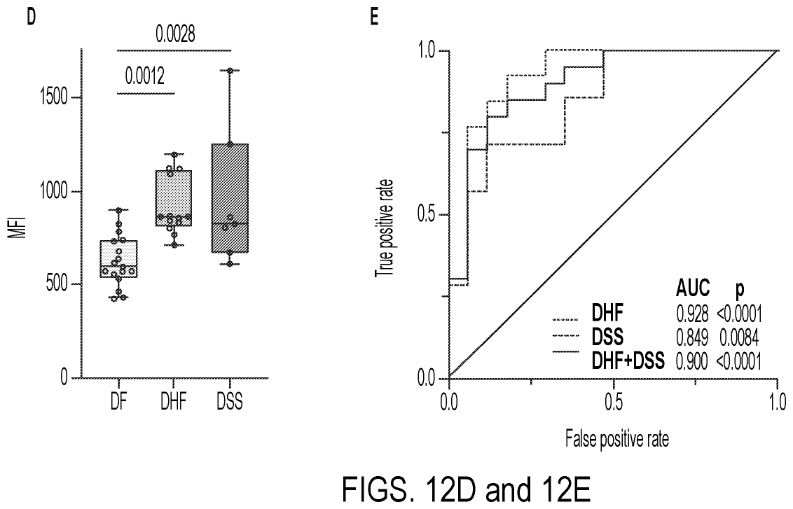

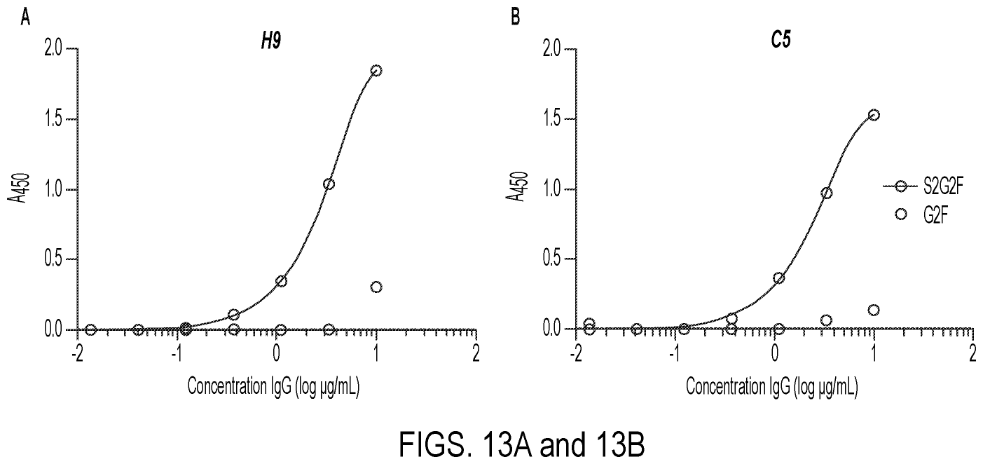

For serum or purified IgG in FIGS. 12A-C, samples were obtained from a previously described patient cohort (T. T. Wang et al., Science 355, 395-398 (2017)). For dengue virus-infected patients in FIGS. 12D-E, purified IgG from a previously published dengue virus-infected cohort was used.

TABLE 5
Data collection and refinement statistics.
Data collection

| Space group | P61 |
|---|---|
| Cell dimensions | |
| a, b, c (Å) | 127.323, 127.323, 100.4 |
| α, β, γ (°) | 90, 90, 120 |
| Resolution (Å) | 110.265-4.71 |
| Rmeas (%) | 14.3 (178.2) |
| I/-(I)I/σ(I) | 6.4 (1.1) |
| CC 1/2 | 99.7 (44.4) |
| Completeness (%) | 98.5 (99.7) |
| Multiplicity | 4 (4.2) |

Refinement

| | |
|---|---|
| No. reflections | 4,782 |
| Rwork/Rfree (%) | 33.95/35.99 |
| No. atoms | |
| Protein | 4,415 |
| B factors | |
| IgG1 Fc | 336.24 (chain A), 348.78 (chain B) |
| B7 | 402.96 (chain C) |
| Solvent | 367.01 |
| R.m.s. deviations | |
| Bond lengths (Å) | 0.012 |
| Bond angles (°) | 1.384 |

### Example 2

Mass Spectrometry Analysis of the Distribution of IgG Subclasses and Fc-Associated Glycoforms

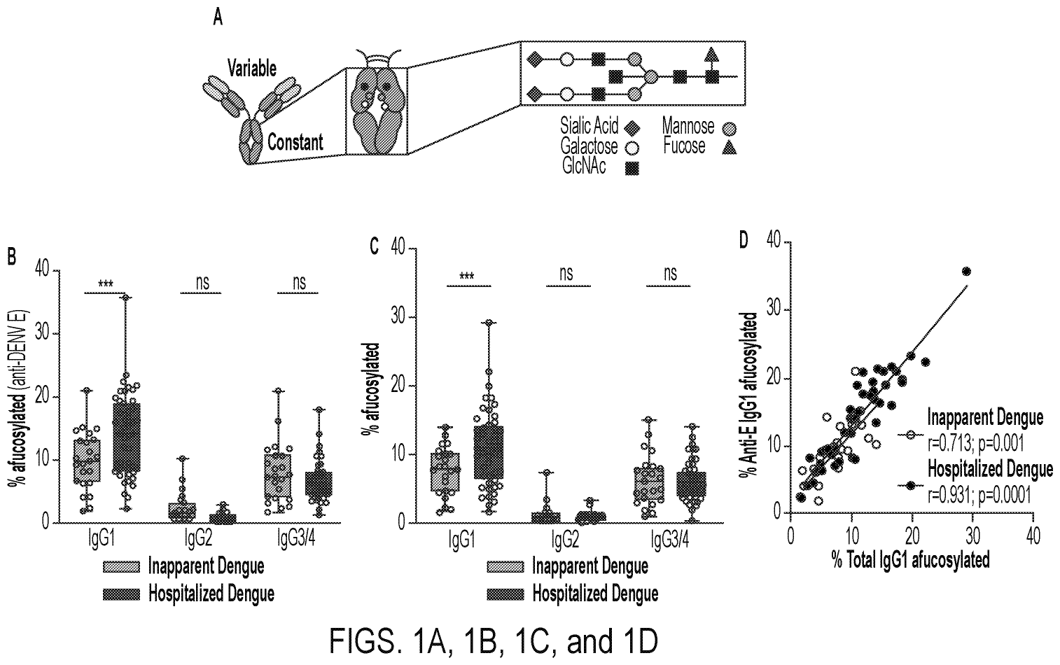

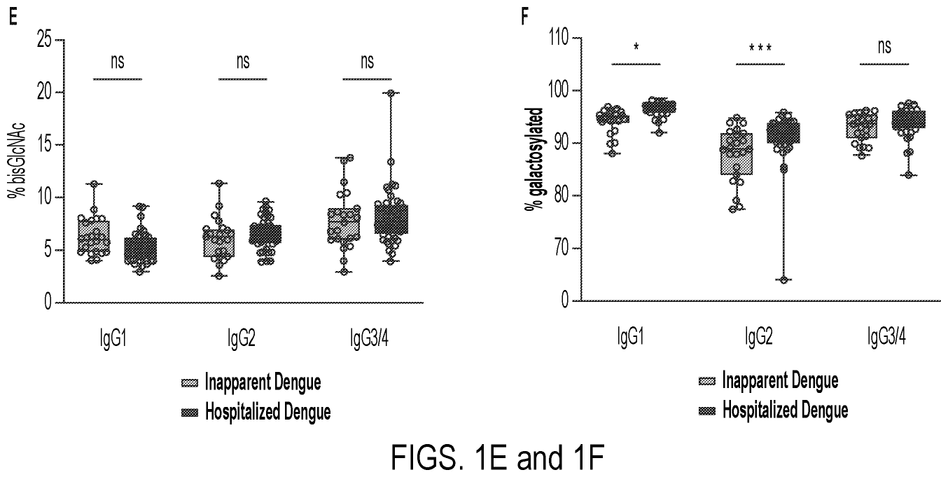

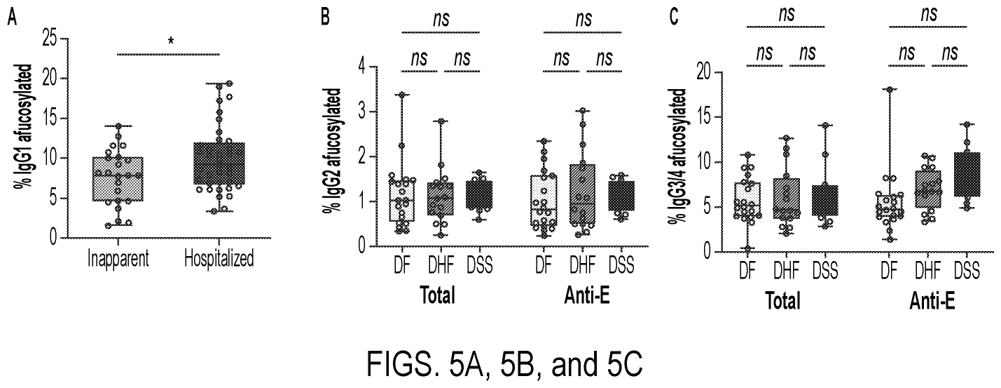

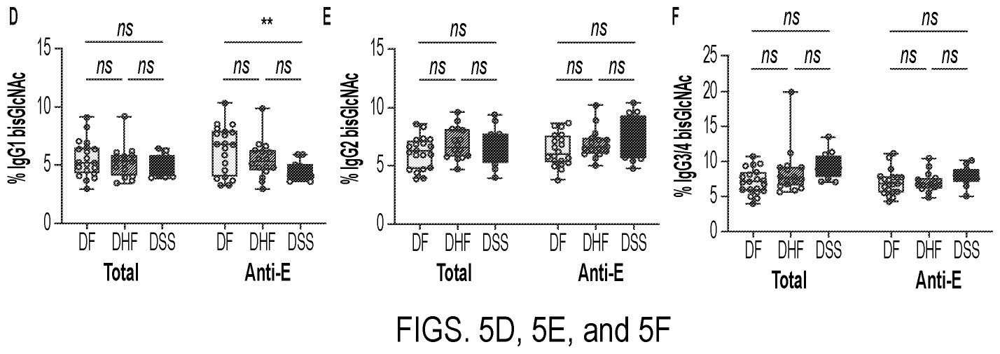

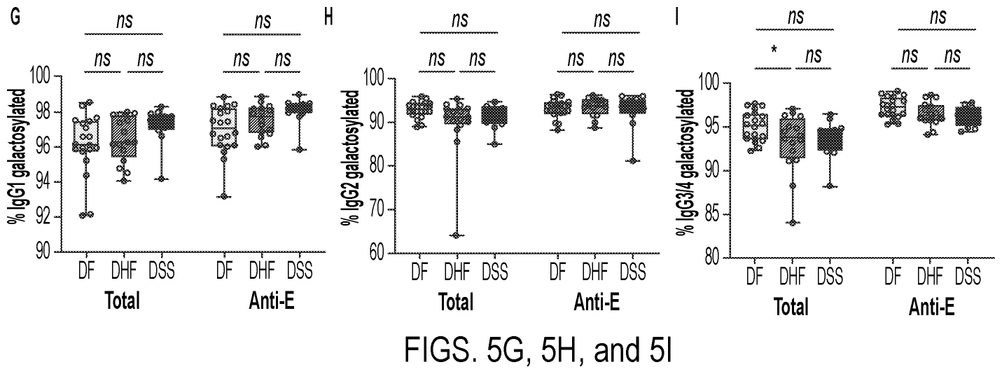

In order to investigate the individual contribution of immune status and IgG Fc glycoforms to the development of severe dengue requiring hospitalization, the distribution of IgG subclasses and Fc-associated glycoforms from individuals with a variable degree of disease severity, ranging from asymptomatic individuals and mildly symptomatic cases (inapparent dengue, n=23 sampled on day 4-9 post-detection) to severe cases of dengue disease that required hospitalization (hospitalized dengue, n=48 sampled at the critical phase of the disease (6-10 d post-symptom onset)) (Table 1), were analyzed by mass spectrometry. Since several factors have been described to influence IgG glycan heterogeneity, including gender, age, and genetic variation among different ethnic groups, all subjects included in this study were from the same geographic region, and the various patient cohorts exhibited comparable age and gender distribution (Table 1). Analysis of the Fc glycan composition of IgGs isolated from these patient groups revealed that hospitalized cases of dengue disease are characterized by a global elevation in the plasma levels of afucosylated IgG1 Fc glycoforms; an effect observed for both antigen-specific (anti-DENV E protein), as well as for total IgGs (FIGS. 1B-D). Elevated levels of IgG1 afucosylation were also observed in hospitalized dengue patients at the time of hospital admission (2-6 d post-symptom onset) (FIG. 5A), indicating that the observed effects are not related to differences in sample timing and are not induced in response to clinical management of hospitalized cases (FIG. 5A).

Differences in the abundance of afucosylated glycoforms among inapparent and hospitalized dengue cases were limited to the IgG1 subclass, as comparable levels of afucosylation of the other IgG subclasses were observed (FIGS. 1B and 1C), indicating the existence of subclass-specific regulatory mechanisms for Fc fucosylation, likely associated with the immunologic conditions that drive IgG class switch (cytokines, T-cell help, nature of antigen (protein vs. carbohydrate) etc.). In addition to afucosylation, hospitalized dengue cases were characterized by elevated levels of IgG1 and IgG2 galactosylation (but not in bisecting GlcNAc)(FIGS. 1E and 1F); however, given the minimal impact of galactosylation on FcγR binding, such differences likely have limited biological significance

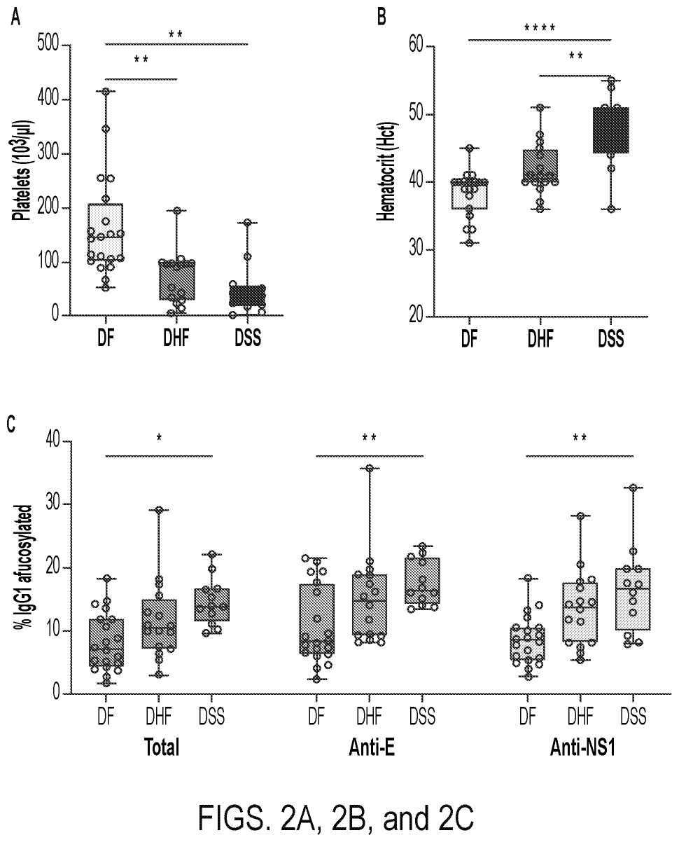

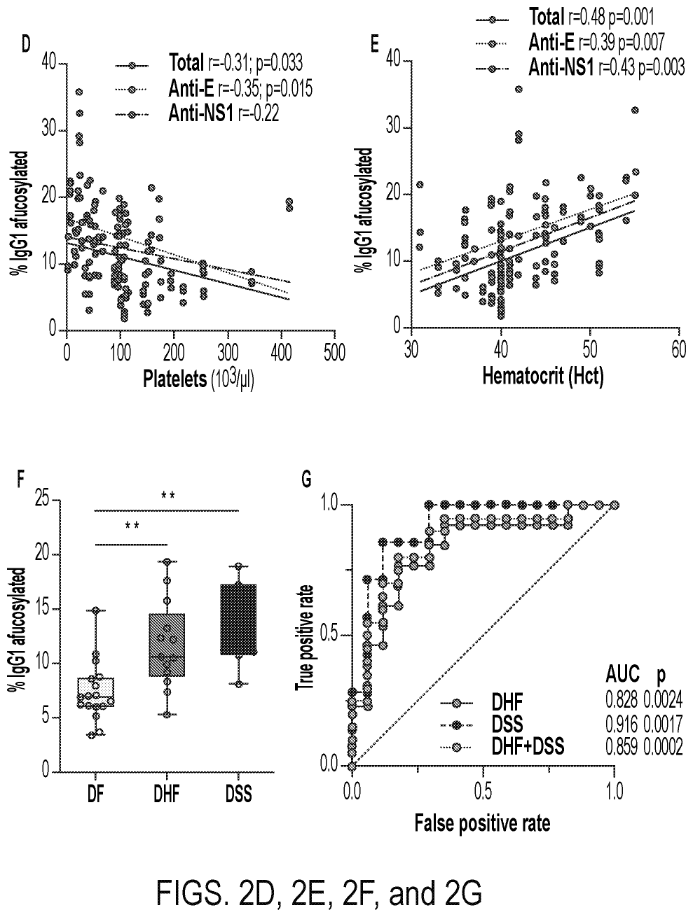

Based on available clinical, biological, and ultrasound data, hospitalized dengue cases were classified according to disease severity using the WHO 1997 classification criteria into dengue fever (DF), dengue hemorrhagic fever (DHF), and dengue shock syndrome (DSS)(24). As expected, disease severity was associated with lower platelet levels and increased hematocrit (Hct), indicating thrombocytopenia and hemoconcentration due to vascular leakage, respectively (FIGS. 2A and 2B). Analysis of the Fc glycan structure of total and DENV-specific IgGs (E protein and NS1) revealed that dengue disease severity is associated with elevated levels of afucosylated IgG1, with severe cases (DSS) characterized by higher levels, whereas milder symptomatic cases (DF) by significantly lower abundance (FIG. 2C). These effects were specific for the IgG1 subclass, and no major differences were noted in the abundance of other Fc glycoforms among the different dengue clinical disease classifications (FIGS. 5B-5I). It was observed that among all hospitalized cases, the abundance of afucosylated IgG1 levels (total and DENV-specific) was inversely correlated with platelet levels, whereas a positive association between IgG1 afucosylation and Hct was seen (FIGS. 2D and 2E), indicating that the abundance of afucosylated IgG1 antibodies represents not only a risk factor for susceptibility to symptomatic disease, but also correlates with the clinical severity of symptomatic dengue disease.

Plasma samples analyzed from hospitalized dengue cases were obtained several days following symptom onset, and it is, therefore, unclear whether or not the increase in afucosylation truly represents a prognostic factor of dengue disease severity or is the outcome of severe disease. To address this issue and provide support on the prognostic value of IgG1 afucosylation in determining susceptibility to severe dengue disease, IgG samples from hospitalized dengue patients obtained at the time of hospital admission (febrile phase; day 2-6 of fever) were analyzed. The analysis revealed that aberrant IgG glycosylation precedes symptom development in severe dengue patients and contributes to disease pathogenesis, as patients that developed DHF or DSS had significantly higher abundance of afucosylated IgG1 glycoforms at admission compared to DF patients (FIG. 2F). The ROC analysis also confirmed that the levels of IgG1 afucosylation at hospital admission are predictive of severe dengue disease (FIG. 2G), contributing to the pathogenesis of severe dengue disease.

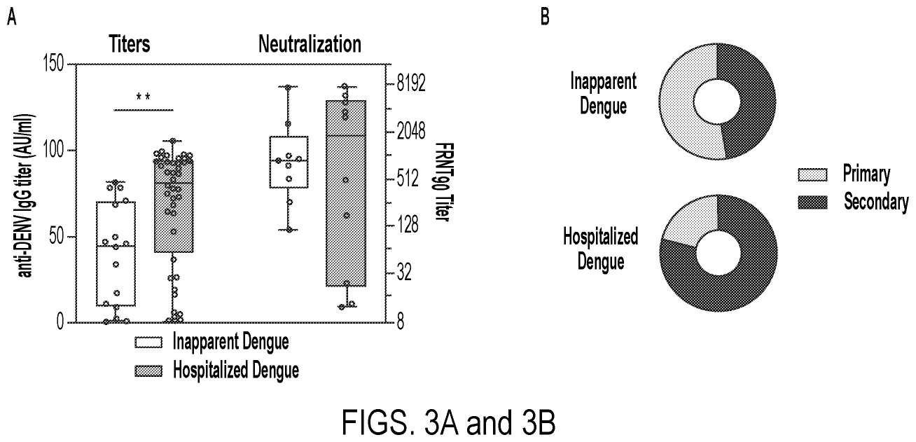

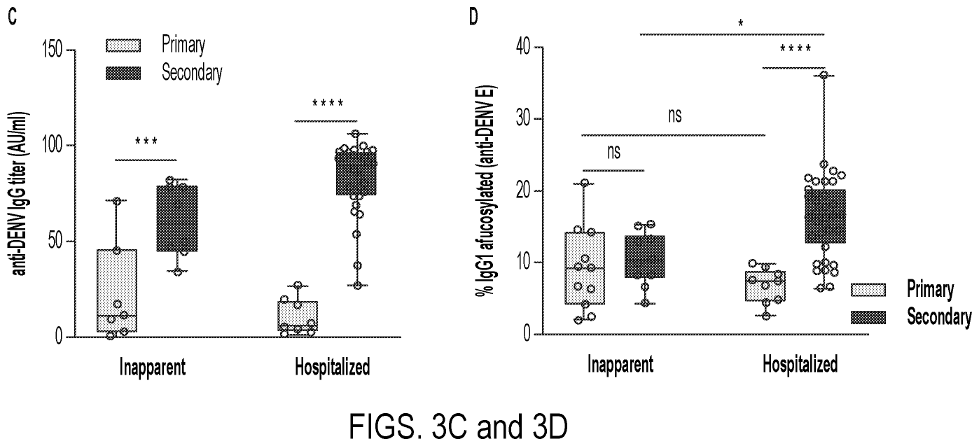

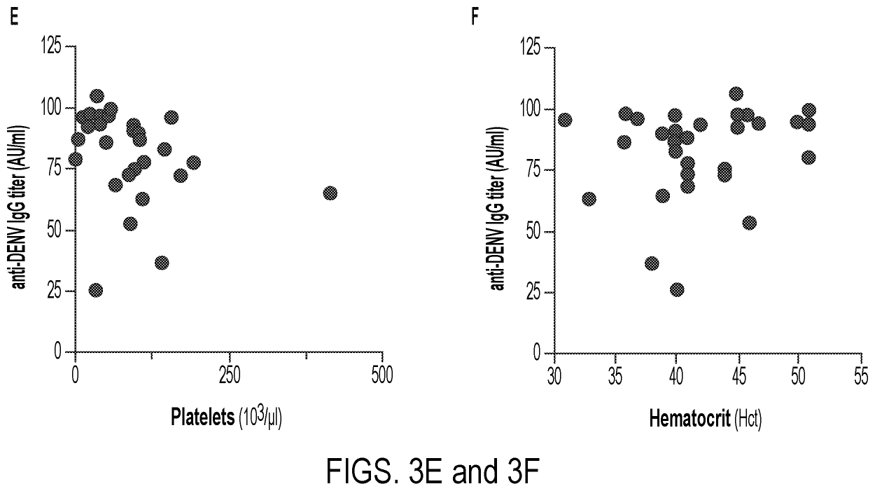

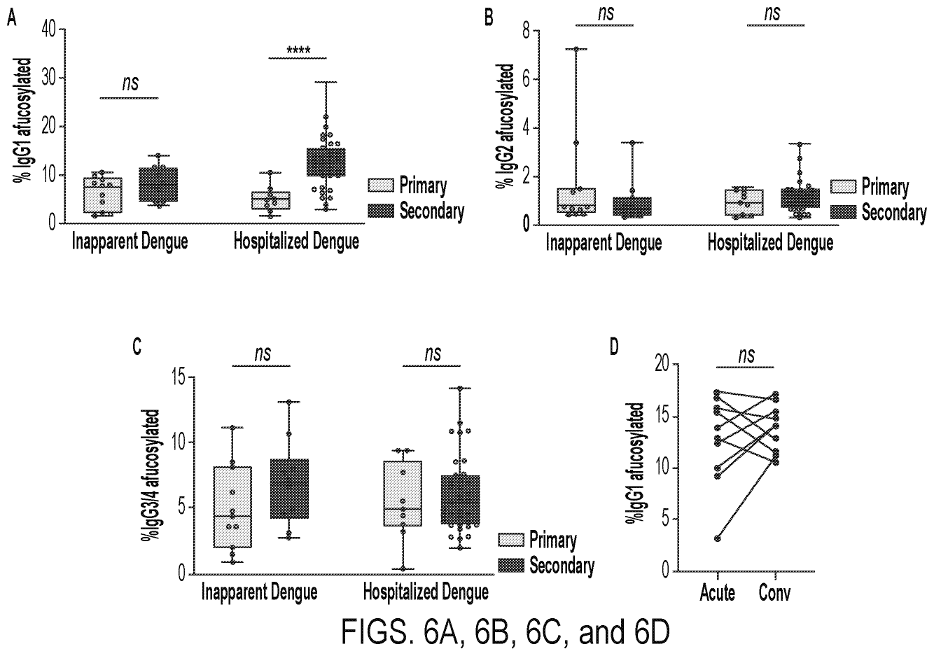

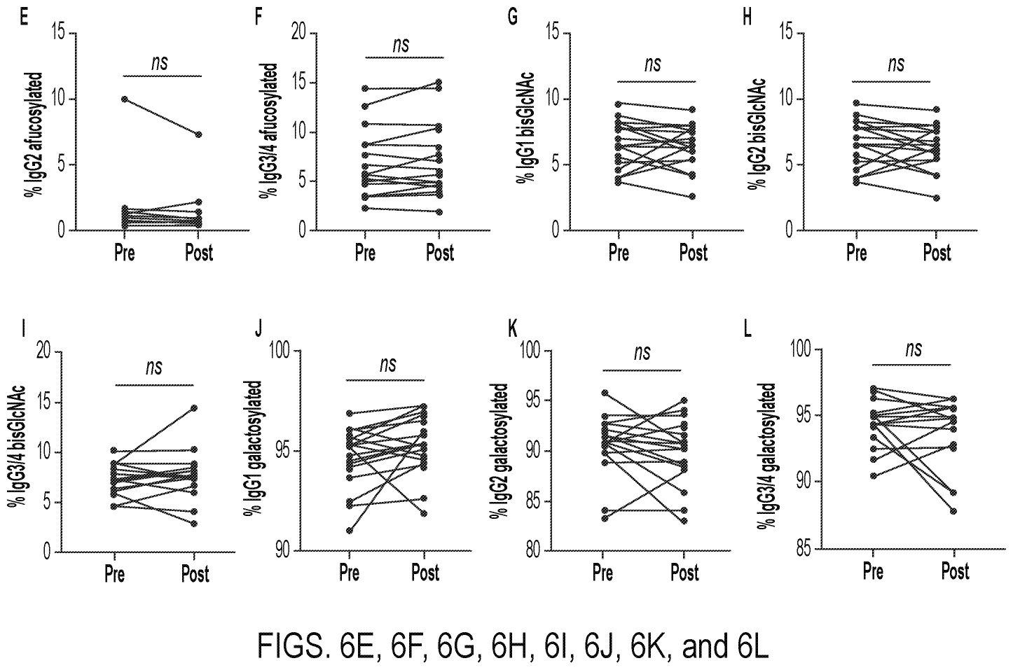

Whether elevated levels of afucosylated IgG1 glycoforms observed in severe dengue cases are associated with prior immune history to DENV or with the titers of anti-DENV IgG in these patients were analyzed. The anti-DENV titers and immune status to DENV in cohorts with inapparent and hospitalized dengue infection were determined. Consistent with prior reports (25, 26), elevated levels of anti-DENV IgGs (FIG. 3A), as well as increased frequency of secondary DENV infection (FIG. 3B) in hospitalized dengue cases, compared to patients with inapparent dengue disease was observed, indicating a pathogenic role of pre-existing anti-DENV IgGs. However, when patients were stratified based on immune history, it became apparent that the elevated anti-DENV titers observed in hospitalized dengue cases were due to the higher frequency of secondary DENV infection in these patients. Indeed, secondary DENV infection was characterized by comparable anti-DENV IgG titers between inapparent and hospitalized dengue cases, indicating that neither the anti-DENV IgG titers nor the DENV immune history alone can sufficiently predict susceptibility to dengue disease (FIG. 3C). In contrast, IgG1 afucosylation was specifically elevated only in hospitalized, but not in inapparent dengue cases with a prior immune history of DENV infection (FIG. 3D; FIGS. 6 A-C). Likewise, while IgG1 afucosylation among hospitalized dengue patients is associated with platelet levels or Hct (FIGS. 2D-2E), no such association was observed for anti-DENV IgG titers (FIGS. 3E-3F). These findings indicate that the IgG1 afucosylation, when combined with DENV immune status, represents a more sensitive and accurate determinant for susceptibility to dengue disease and is associated with clinical severity of symptomatic dengue disease.

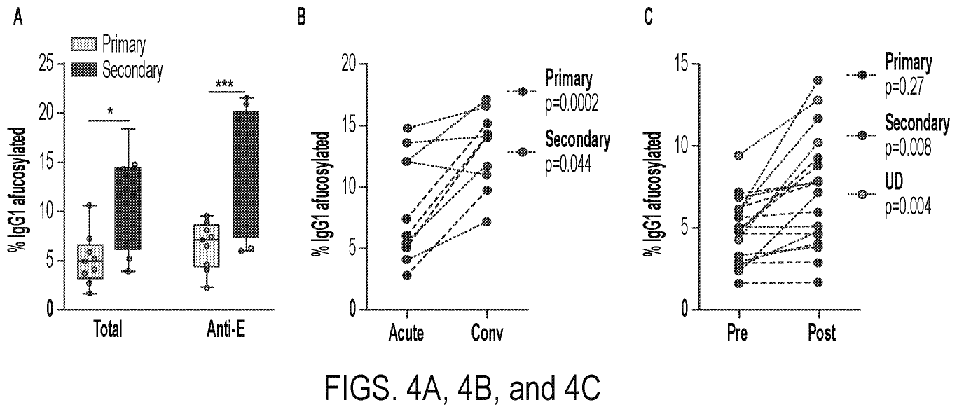

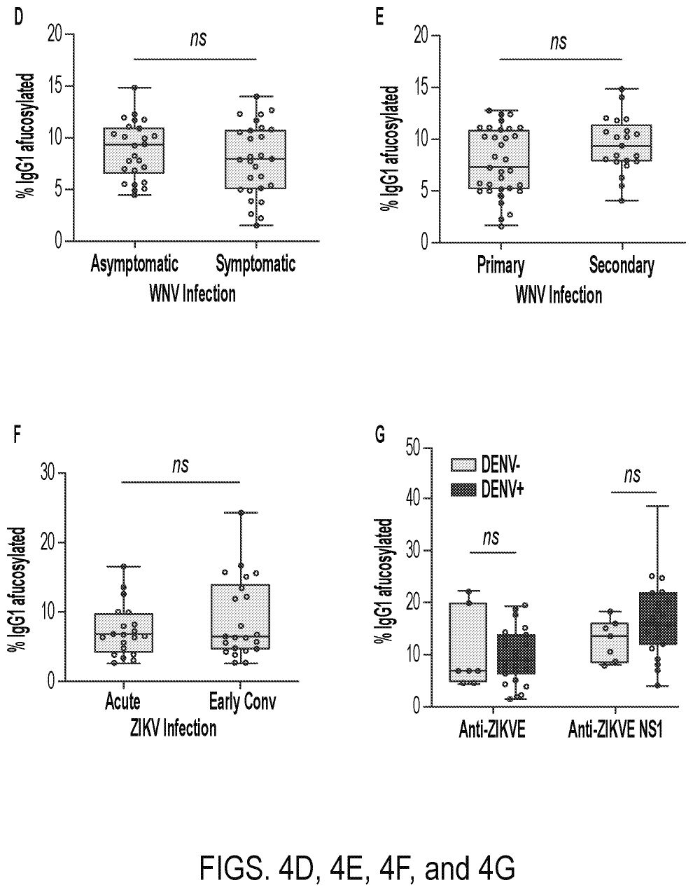

Although the instant disclosure indicates that afucosylated IgG1 is specifically elevated in secondary DENV infection, it is unknown whether it is the severity of the disease that is inducing higher afucosylation or afucosylated IgG antibodies are elicited upon secondary DENV exposure. To address this hypothesis, hospitalized dengue patients with identical clinical classification (DF) were compared for the levels of IgG1 afucosylation among patients with differential DENV immune history (naïve or DENV-experienced). It was observed that DF patients with prior DENV exposure were characterized by elevated levels of IgG1 afucosylation, indicating that it is the immune history, rather than disease severity, that determines the fucosylation status of IgG1 antibodies (FIG. 4A). Consistent with these findings, analysis of the same DF cohort at convalescence (day 23-100 after symptom onset) revealed that primary DF cases exhibit significantly elevated levels of afucosylated IgG1 glycoforms at convalescence compared to the acute phase of infection; an effect that was not observed in secondary DF cases or in DHF/DSS cases (entirely secondary cases), which were characterized by persistently high levels of IgG1 afucosylation during both the acute, as well as the convalescent phase (FIGS. 4B and 6D).

Fc glycosylation is dynamically regulated during an immune response with specific Fc glycoforms becoming enriched upon vaccination or during certain inflammatory diseases or infections. Although the determinants that regulate Fc fucosylation are poorly characterized, it is likely that the observed elevation in the levels of IgG1 afucosylation in secondary dengue disease patients might reflect specific modulation by DENV infection of the pathways that regulate Fc fucosylation. To test whether secondary DENV infection has the capacity to impact IgG1 afucosylation, 18 individuals were included in this study (with either primary or secondary DENV immune status) from whom blood samples were obtained 4 days before and 7-9 days after confirmed (by qRT-PCR) DENV infection (Table 1). Analysis of matched pre- and post-infection plasma IgGs revealed that afucosylated IgG1 glycoforms are specifically induced following DENV infection (FIG. 4C) mainly in the subset of subjects with prior DENV immune history. In contrast, no changes were noted in the levels of afucosylation of other IgG subclasses, as well as in other glycan modifications, like galactosylation and bisection (FIGS. 6E-L), indicating that DENV infection specifically modulates IgG1 afucosylation, without inducing any global, non-specific changes in IgG Fc glycosylation.

These findings on dengue patients indicate that secondary DENV infection impacts the levels of IgG1 afucosylation, which in turn modulates susceptibility to severe symptomatic dengue disease. However, it is unknown whether these effects are specific for DENV or extend to other flaviviruses, like West Nile (WNV) and Zika (ZIKV) viruses. The Fc glycan structure of IgGs from WNV patients (n=54) with either asymptomatic or symptomatic disease and differential immune status (primary vs. secondary WNV infection, as determined by IgG/IgM ratio; immunity against other flaviviruses was assessed to ensure that secondary WNV cases do not represent prior flavivirus infection) were analyzed (32) (FIGS. 7A-D; Table 2). In contrast to dengue patients, no difference in the levels of IgG1 afucosylation was observed among WNV patients with asymptomatic or symptomatic disease (FIG. 4D). Likewise, prior exposure to WNV was not associated with increased abundance of IgG1 afucosylation (FIG. 4E). To determine whether ZIKV infection was associated with an increase in the levels of IgG1 afucosylation, the Fc glycan structure of IgGs from serum samples obtained from ZIKV infected patients (defined as ZIKV RNA⁺) at the acute phase of infection (n=20; ZIKV RNA⁺, anti-ZIKV IgM⁻) and at early convalescence (n=21; anti-ZIKV IgG/IgM+) were compared (Table 3). It was observed that comparable IgG1 afucosylation levels in IgGs from ZIKV-infected patients obtained at the acute and early convalescence, indicating that in contrast to DENV, ZIKV infection has no impact on Fc fucosylation (FIG. 4F; FIGS. 7E-H).

Widespread ZIKV and DENV infections co-exist in endemic tropical areas, raising the possibility that prior exposure to DENV might lead to a global dysregulation of IgG1 afucosylation, which in turn might result in higher abundance of afucosylated Fc glycoforms at baseline, as well as upon antigenic exposure to DENV or ZIKV. To investigate the impact of pre-existing anti-DENV immunity on the Fc glycosylation of IgGs elicited upon ZIKV infection, the Fc glycan structure of ZIKV-infected patients with a differential immune history of DENV infection were analyzed (Table 4). Analysis of anti-ZIKV E and NS1 IgGs purified from convalescent plasma samples revealed comparable levels of afucosylated IgG1 Fc glycoforms among DENV-naïve or experienced patients (FIG. 4G). Likewise, no differences in the abundance of other Fc glycoforms were observed in ZIKV patients with a differential immune history of DENV infection (FIGS. 8A-D).

Fc glycosylation represents a key determinant for the affinity of the Fc domain for the various FcγRs, and even small changes in the structure and composition of the Fc glycan have a significant immunomodulatory impact. Several studies on IgG function have previously determined that the Fc glycan structure is dynamically regulated during an immune response and specific Fc glycoforms become enriched following vaccination or infection, as well as in chronic inflammatory responses. For example, increased abundance of afucosylated IgG1 glycoforms has been reported following infection with enveloped viruses, including HIV and SARS-CoV-2. Although the mechanisms that regulate Fc domain glycosylation remain poorly characterized, previous studies have determined that in the context of vaccination, antigen exposure differentially regulates the expression and activity of glycosyltransferases in the various B-cell subsets, which in turn modulates the addition of specific saccharide units to the Fc-associated glycan structure. It is therefore anticipated that during an immune response, B cells and plasma cells become exposed to distinct immunological mediators that dynamically regulate Fc domain glycosylation and account for the enrichment of certain Fc glycoforms, like afucosylated Fcs, upon vaccination or during disease.

In the context of dengue disease, the present findings indicate that DENV infection has a profound impact on Fc glycosylation, by specifically inducing afucosylation of IgG1 antibodies, whereas it has no effects on other glycoforms. This data supports that in addition to antigen-specific IgG1, severe dengue patients are characterized by a global increase in IgG1 afucosylation, indicating that non-antigen-specific afucosylated IgGs, that are present in excess, exert competition effects that could limit the protective or pathogenic Fc effector activity of anti-DENV IgGs. However, due to the nature of the IgG1-FcγRIIIa interaction, competition from bulk serum IgG is expected to be minimal, as FcγRIIIa is a low-affinity receptor for IgG1 (either fucosylated or afucosylated) and cannot be engaged by monomeric IgG1. Instead, FcγRIIIa crosslinking occurs only via multiple low-affinity, high-avidity interactions that trigger receptor clustering and downstream signaling. Therefore, the binding strength of IgG immune complexes (as in the case of anti-DENV IgG complexed to DENV virions) is expected to be several orders of magnitude greater than monomeric, non-antigen-specific IgG, overcoming any potential competition effects. Indeed, when the in vivo cytotoxic activity in FcγR humanized mice of cytotoxic anti-platelet mAbs in the presence of excess, non-antigen-specific IgG, were evaluated, it was observed that their cytotoxic activity was not influenced by the presence of excess irrelevant fucosylated or afucosylated mAb, indicating that competition effects from serum IgG (even if afucosylated) are expected to be minimal (FIG. 8E).

It is likely that DENV infection elicits unique inflammatory cues, which in turn shape B cell responses that are characterized by aberrant induction of afucosylated IgG1 antibodies. In addition to indirect effects, DENV could have direct immunomodulatory consequences via infection of B cells. For example, scRNAseq analysis of PBMCs from dengue patients has previously determined that B cells can be efficiently infected by DENV with a significant fraction of them being positive for viral RNA. Infection of B cells by DENV could modulate Fc glycosylation by inappropriate activation of cellular antiviral responses, as well as dysregulated B cell selection, survival, and differentiation, thereby accounting for elevated serum levels of afucosylated IgG1 glycoforms. Analysis of convalescent plasma samples from recovered dengue patients revealed persistently high levels of IgG1 afucosylation, indicating that dengue infection has profound consequences on the Fc glycan structure of IgG antibodies that last several weeks post-infection. Since increased abundance of afucosylated IgG1 glycoforms has been associated with increased risk for autoimmunity, it is likely that elevated levels of IgG1 afucosylation might put dengue patients at risk for developing autoimmune pathologies. Indeed, previous studies have shown that severe dengue disease is associated with the presence of autoantibodies against platelets and endothelial cells, as well as against coagulation proteins, whereas a large population-based cohort study reported higher incidence of several autoimmune diseases in symptomatic dengue patients compared to control subject). These findings, along with the data presented in the present application, indicate that dysregulated IgG1 fucosylation during dengue infection, not only represents a risk factor for developing severe dengue disease, but might also be associated with increased susceptibility to autoimmune disorders, which are often characterized by elevated afucosylation levels.

The comparative analysis on Fc glycosylation in ZIKV and WNV-infected patients revealed that the increased abundance of afucosylated IgG1 glycoforms that is associated with symptomatic disease is restricted to DENV and does not extend to other flaviviruses. DENV, ZIKV, and WNV are all mosquito-transmitted RNA viruses of the flavivirus genus; however, in contrast to DENV, for which substantial experimental and epidemiological evidence indicates the contribution of ADE mechanisms in modulating disease pathogenesis, the role of pre-existing IgGs in driving disease susceptibility to symptomatic ZIKV and WNV remains elusive. For example, recent studies have shown that anti-ZIKV monoclonal antibodies or highly cross-reactive flavivirus-immune plasma samples with neutralizing activity against ZIKV have the capacity to mediate ADE of ZIKV infection in vitro, whereas passive administration of flavivirus-immune plasma at sub-neutralizing doses exacerbates disease severity upon ZIKV challenge in mouse disease models. However, these findings have not been replicated in studies using non-human primates, which failed to demonstrate evidence for ADE of ZIKV infection. Similarly, epidemiologic studies in humans revealed no correlation of viral load and cytokine response during ZIKV infection with prior flavivirus immunity. Since the observed effects on Fc glycosylation were evident only in DENV-, but not in ZIKV- or WNV-infected patients, alterations in Fc fucosylation likely represent an immune evasion mechanism that is unique to DENV, which drives disease pathogenesis through specific modulation of the capacity of anti-DENV IgG antibodies to interact with FcγRs and mediate ADE of dengue disease.

Although prior immune history to DENV has been proposed to be a major risk factor for progression to symptomatic dengue disease, anti-DENV titers alone cannot predict susceptibility to severe dengue disease, as the majority of DENV-experienced patients still develop asymptomatic or mild symptomatic disease upon re-infection. Instead, the present findings support that dengue infection causes a specific increase in the abundance of afucosylated IgG1 glycoforms and the afucosylation status of IgG1 antibodies when combined with information on DENV immune history, represents a robust prognostic tool for predicting risk to develop severe symptomatic dengue disease upon hospitalization. More importantly, afucosylated IgG1 levels are not only associated with susceptibility to symptomatic disease, but are also correlated with the specific clinical manifestations of severe dengue disease. In summary, the present findings support a key role for the Fc glycan structure in mediating ADE of DENV disease and indicate that analysis of the abundance of afucosylated Fc could predict susceptibility for severe dengue disease in high-risk patient groups, guiding the development of approaches to prevent or reduce disease-associated clinical manifestations.

### Example 3

Nanobody Probes for Detecting Specific IgG Fc Glycoforms Provide Rapid Prognostic Tools for Acute Viral Infections

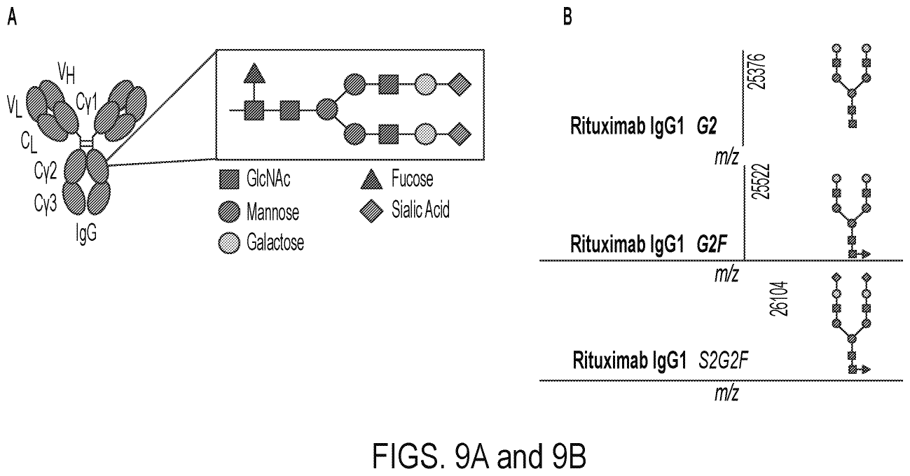

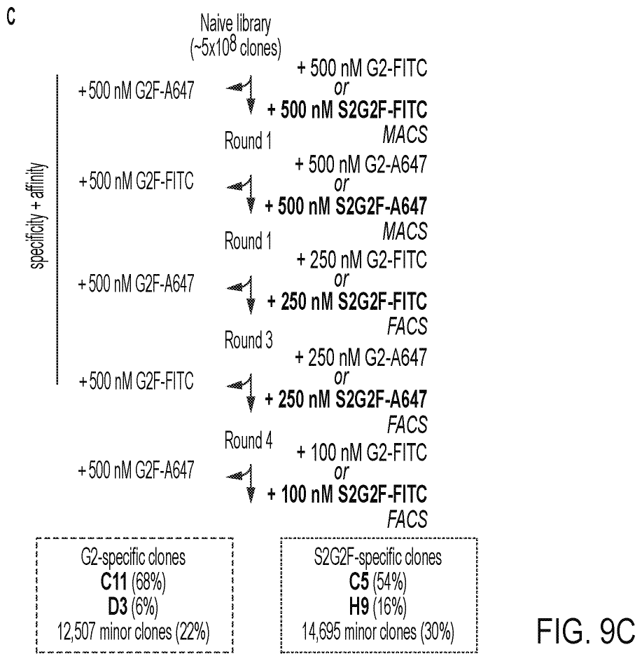

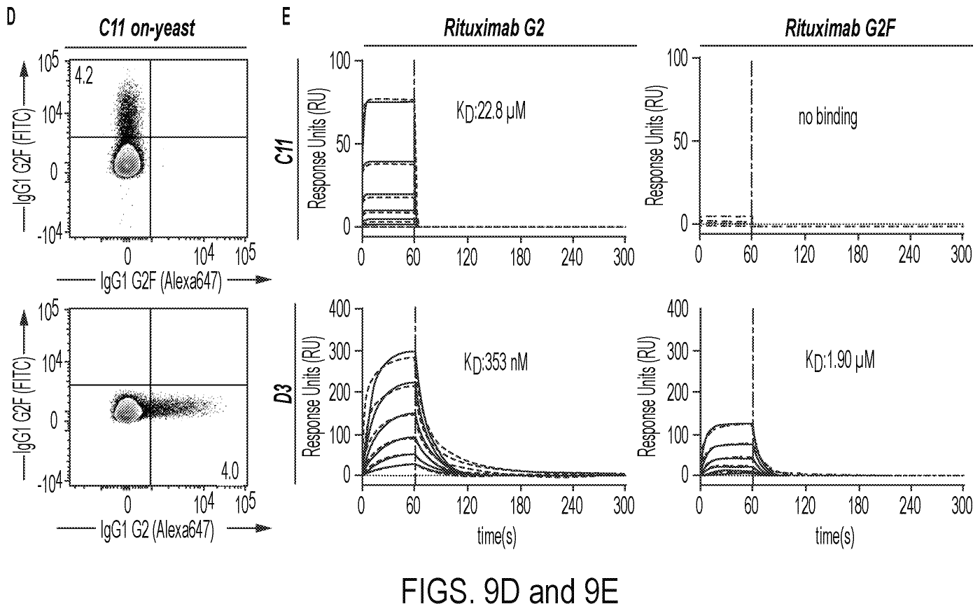

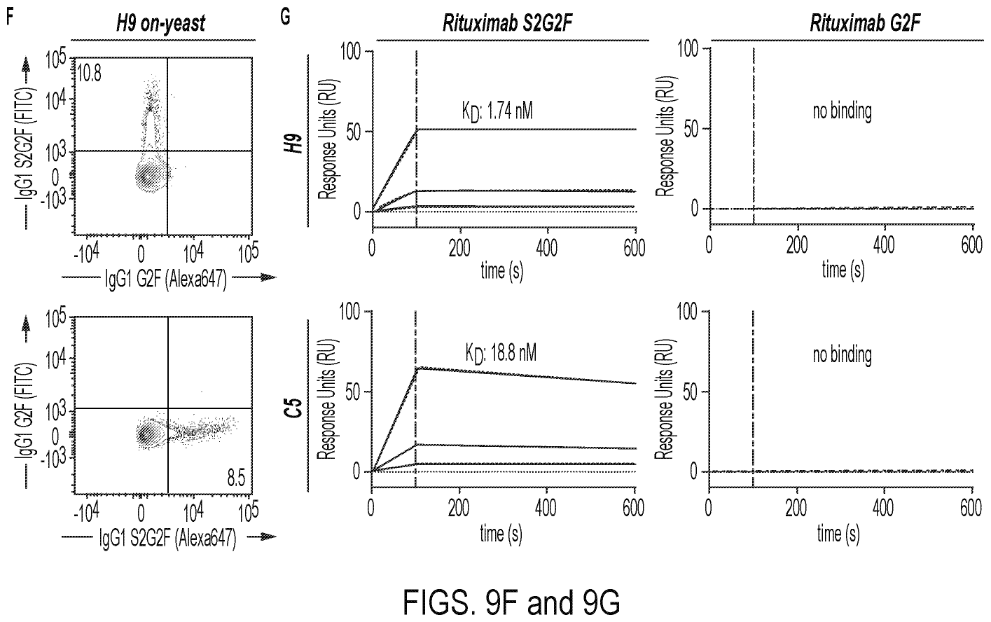

The structure of the immunoglobulin G (IgG) Fc domain is a key determinant of antibody effector function. Both the peptide backbone sequence and the complex, biantennary N-linked glycan on Asp297 (FIG. 9A) influence the affinity and selectivity of Fc-Fc gamma receptor (FcγR) interactions, thereby dictating the protective or pathogenic activity of antibodies. More specifically, IgG lacking its core fucose residue has ˜10-20-fold higher affinity for the activating FcγRIIIA, while terminal sialylation allows for engagement of Type II FcRs. While it is well established that Fc glycan modifications are dynamically regulated both in health and disease, recent reports have provided support for the role of these modifications as prognostic indicators of disease progression in viral illness. In dengue virus-positive patients, levels of afucosylated IgG1 antibodies at admission predict whether a patient will progress to severe disease, namely dengue hemorrhagic fever (DHF) or dengue shock syndrome (DSS). This same modification also stratifies and serves as a prognostic indicator of clinical severity in PCR-positive COVID-19 patients. Serum IgG Fc glycoforms are typically studied using highly accurate but labor-intensive and costly methods such as electrospray ionization mass spectrometry (ESI-MS) and high-performance liquid chromatography (HPLC). Though additional high-throughput methods have been proposed, they are neither accurate nor sensitive enough nor suited for clinical, point-of-care deployment. These methodological barriers have limited the integration of these promising biomarkers into general use, necessitating a more efficient and scalable approach. Further, because the abundance of afucosylated IgG has predictive power in dengue virus and SARS-CoV-2 infection, a probe for this glycoform would open the door for rapid point-of-care tools that could be used to stratify patient risk based on disease-related changes to the IgG Fc glycome.

Nanobodies are used as both therapeutic agents and diagnostic probes due to their small size, ease of production, and excellent specificity and affinity. Derived from camelid species, they share a similar molecular architecture with human and mouse immunoglobulin variable-heavy chain (VH) domains, with four conserved framework regions surrounding three hypervariable complementarity determining regions (CDRs). However, the CDR3 in most camelids is substantially longer than that of mouse or human variable regions, enabling greater structural flexibility for recognition of recessed or otherwise inaccessible epitopes, as may be the case with the N-linked Fc glycan. To capitalize on these advantages, a synthetic yeast nanobody display library that approximates camelid nanobody diversity in vitro was utilized.

To precisely select for nanobodies specific for afucosylated and sialylated IgG Fc, clinical grade rituximab was chemoenzymatically engineered into its galactosylated afucosylated (G2), galactosylated fucosylated (G2F), or a galactosylated sialylated fucosylated (S2G2F) glycoforms (FIG. 9B). The three glycoforms were fluorescently labeled with FITC and Alexa647 and yeast displaying nanobodies with specific affinity for the G2 or S2G2F glycoforms were selected through two rounds of magnetic enrichment (MACS) and three rounds of fluorescence-activated cell sorting (FACS)-based enrichment (FIG. 9C). High affinity clones were obtained by successively lowering the target glycoform concentration, while specificity was maintained throughout each round by counter-selecting against a high fixed concentration of the undesirable G2F glycoform. After the final round of selection, the resulting library was sequenced and single yeast clones were characterized by flow-cytometry (FIGS. 9D and 9F). This screening strategy yielded two nanobodies specific for the G2 glycoform (C11, D3) and two nanobodies specific for the S2G2F glycoform (C5, H9) (FIGS. 9D-G). Although D3 bound the G2 glycoform with higher affinity than C11 (KD=323 nM vs. 22.8 μM), affinity for the G2F glycoform was demonstrably higher (KD=1.9 μM vs. n.b.). Based on these properties, affinity mature C11 was further pursued. Sialylated IgG Fc-specific clones H9 and C5 were sufficiently high affinity (KD=1.74 nM and 18.8 nM) and did not require further improvement (FIGS. 1F-G).

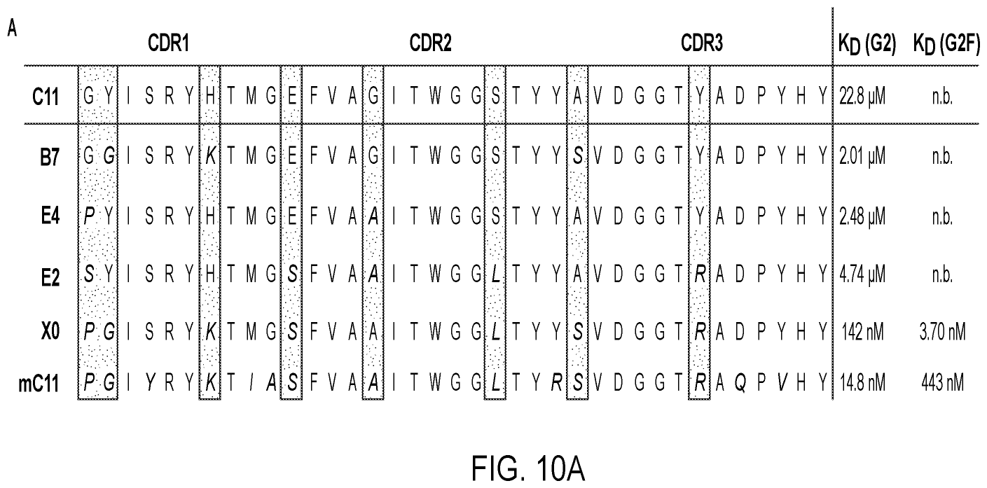

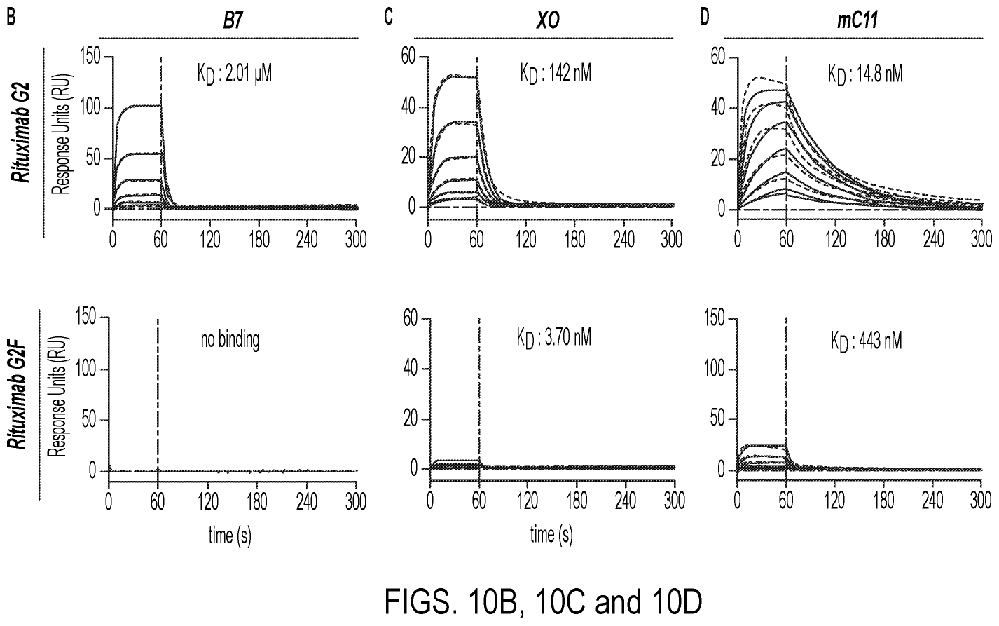

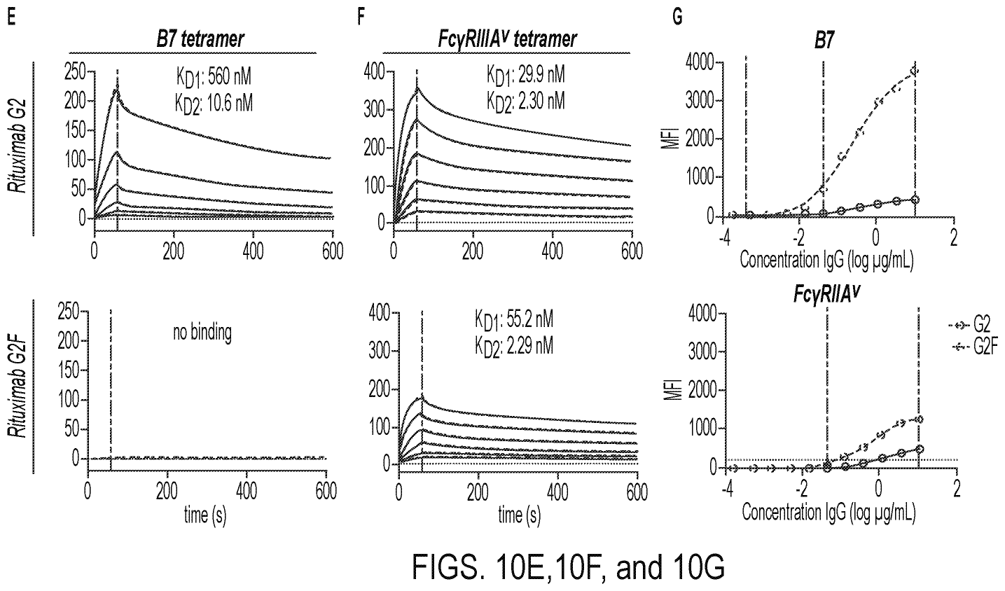

To further affinity mature clones specific for afucosylated IgG, a site-saturation mutagenesis library of the CDRs of C11 was designed. Two rounds of selection of the resulting library, in which G2F was maintained in 50-fold molar excess of G2 bait, yielded numerous clones with penetrant mutations at specific ‘hotspots’ within each CDR. These clones demonstrated 10 to 1000-fold affinity for G2 while retaining similar levels of specificity as C11 (FIGS. 10A-C). Combinatorial assembly of the mutations present in the top clones resulted in a dominant clone, mC11, which exhibited a 1000-fold improvement in affinity for G2 when compared to the C11 parental clone at the cost of marginal specificity (FIG. 10D). Based on its higher specificity, clone B7 was chosen and further engineered for increased affinity. Nanobody multimers have been shown to possess drastically higher binding affinities, largely through avidity. To take advantage of this property, biotin-streptavidin tetramers of the most specific nanobody clone, B7, were generated. Notably, tetramerization greatly enhanced binding affinity for G2 (KD1=560 nM, KD2=10.6 nM), while preserving specificity (FIG. 2E).

Though some have proposed the use of soluble Fcγ receptor IIIA (FcγRIIIA) as a detection reagent for afucosylated IgG due to its higher affinity for these glycoforms, B7 tetramers demonstrated much greater specificity by SPR as well as greater sensitivity in immunoassays (FIGS. 10E-F), demonstrating the advantages of the nanobody approach.

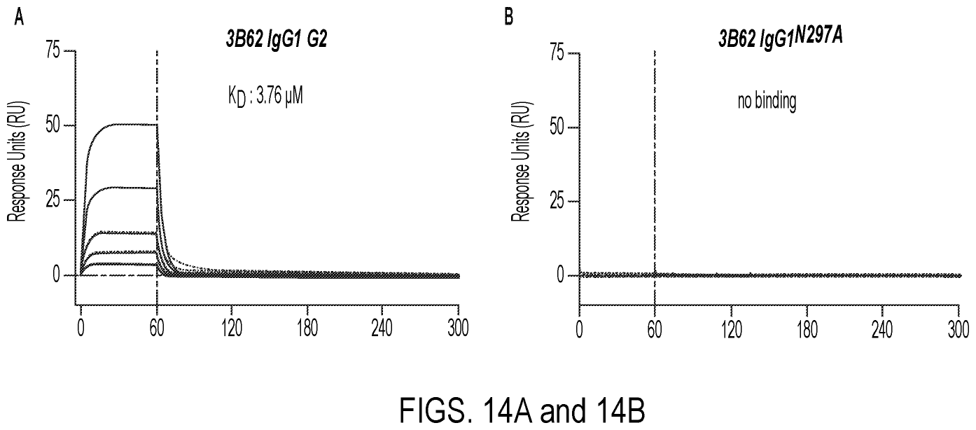

Antibodies and lectins specific for glycan residues are ubiquitous in research. To determine the specificity of B7 and confirm that it recognizes not only the N-glycan but also the IgG protein backbone, an N-linked glycan array was performed using B7 as a probe. As expected, B7 only recognized the human IgG positive control and did not bind any of the N-glycans, regardless of fucosylation status. The specificity of the glycan array was confirmed using the fucose-binding lectin Aleuria Aurantia Lectin (AAL). Expectedly, binding to B7 of the aglycosylated N297A IgG1 mutant abolished all binding, confirming its dependency on the glycan (FIGS. 14A-B).

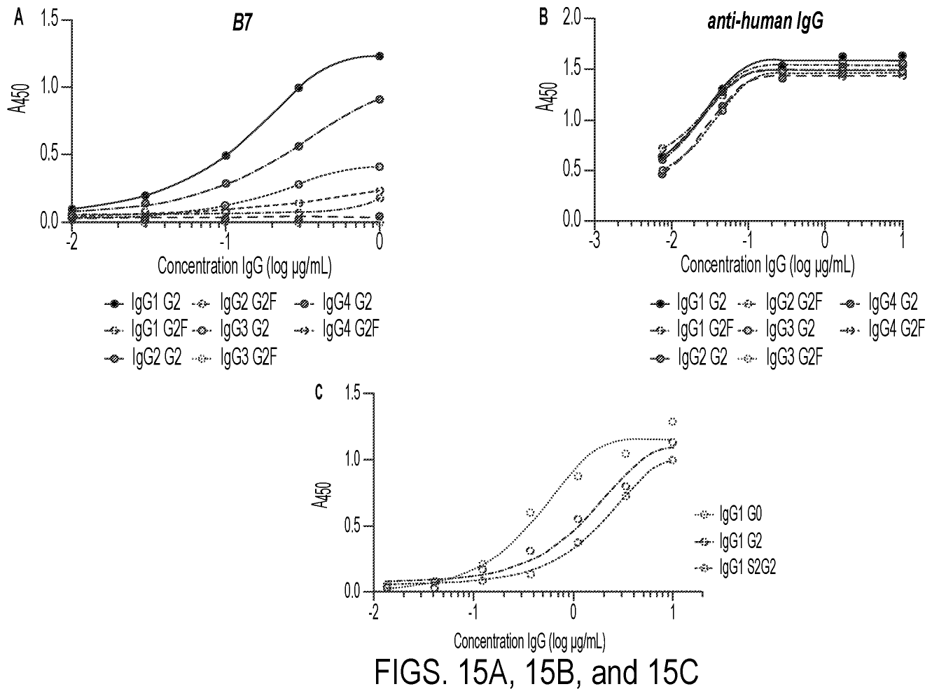

Human IgG is comprised of four subclasses-IgG1, IgG2, IgG3, and IgG4-which share over 90% homology within their Fc domain. To test the subclass specificity of B7, G2 and G2F glycoforms of 6A6, an anti-mouse platelet glycoprotein IIb mAb formatted with a human IgG1-4 Fc domain were used. Because the library screening strategy used rituximab, a human IgG1 mAb, B7 exhibited preferential binding (IgG1>IgG2>IgG3>>IgG4) (FIGS. 15A-B). However, specificity for afucosylated glycoforms was maintained across all subclasses, with the largest fold-change in specificity for IgG1 and IgG2. In contrast to IgG1, specific glycoforms of other subclasses have a limited biological role in disease, either due to their low abundance in serum or weak FcγR binding. Further, only afucosylated IgG1 has been correlated with the clinical course of inflammatory diseases, while analysis of afucosylated glycoforms of IgG2-4 has demonstrated insignificant predictive power. Finally, it was verified that B7 retains binding to all afucosylated forms of IgG1 (GO, G2, and S2G2) regardless of galactosylation or sialylation, demonstrating its specificity for all glycoforms lacking the core fucose residue (FIG. 15).

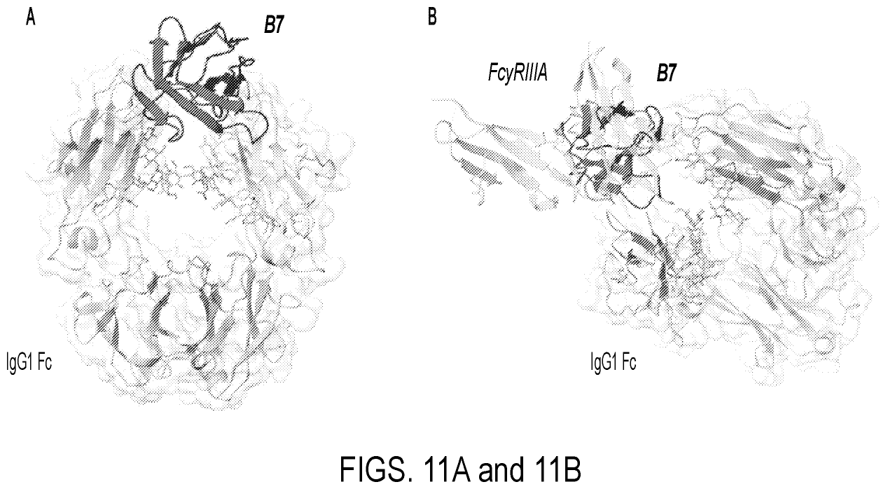

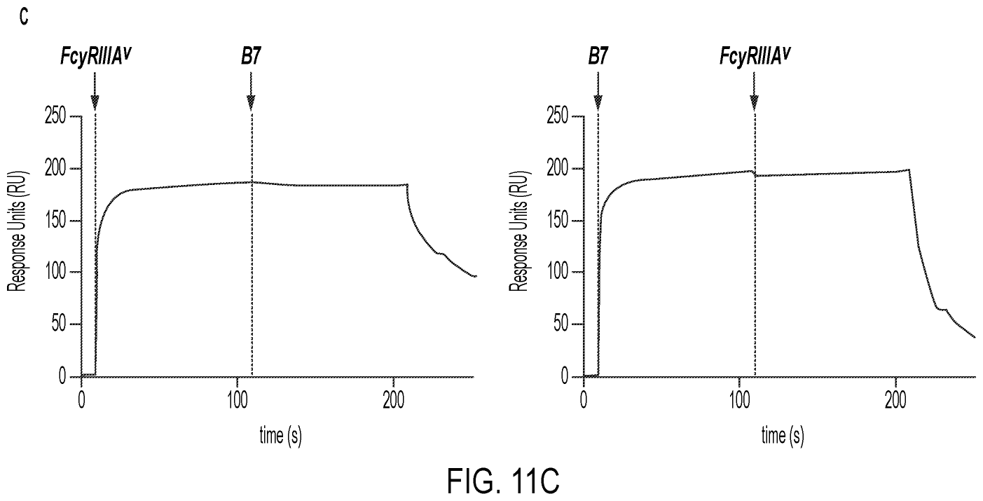

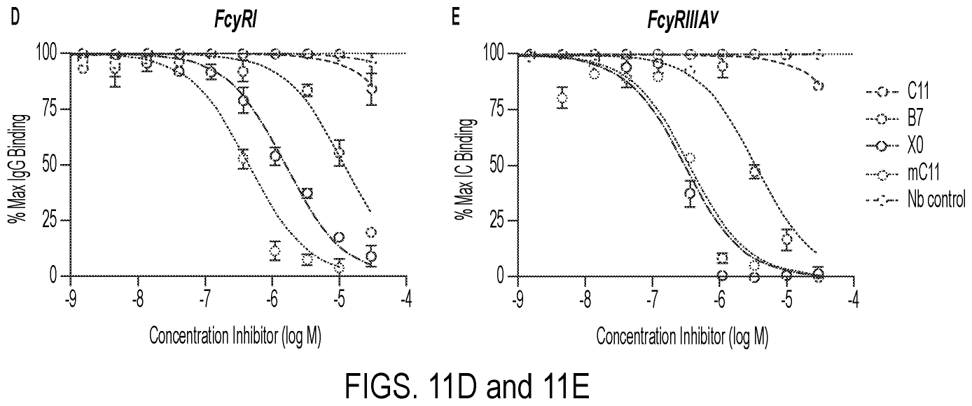

To better understand how B7 discriminates between fucosylated and afucosylated IgG glycoforms, B7 with afucosylated IgG1 Fc were co-crystallized. X-ray data collection and subsequent refinement yielded a structure of the B7-IgG1 Fc complex (FIG. 11A and Table 5). Superimposition of this structure with those of previously published IgG1 Fc-FcγR complexes (PDB 6EAQ, 3SGK, 5VU0) revealed that B7 occupies a similar epitope as FcγRs with asymmetrical binding and 1:1 stoichiometry at the C72-hinge interface (FIG. 11B). Mutually exclusive binding of B7 and FcγRIIIa was further confirmed by SPR epitope mapping experiments (FIG. 11C). Similarly, clones B7, X0, and mC11 were capable of blocking FcγR binding of monomeric IgG or pre-formed immune complexes, indicating direct competition for IgG binding (FIG. 11D).

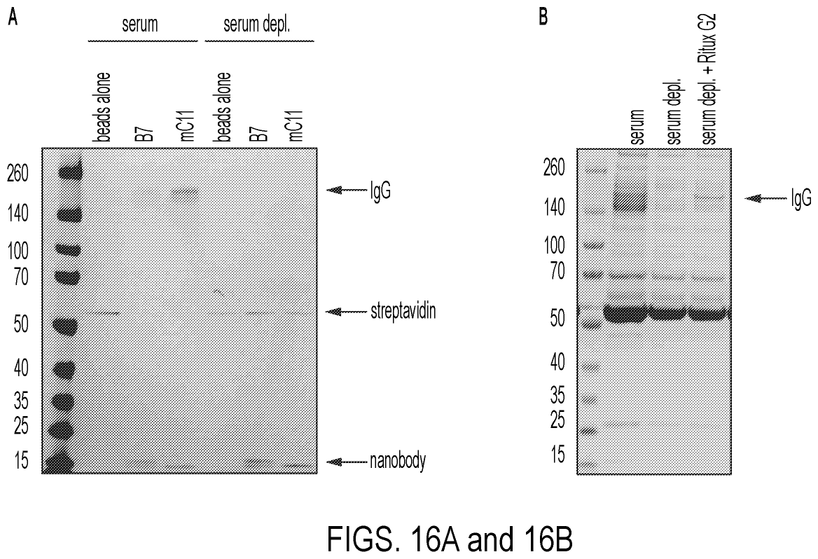

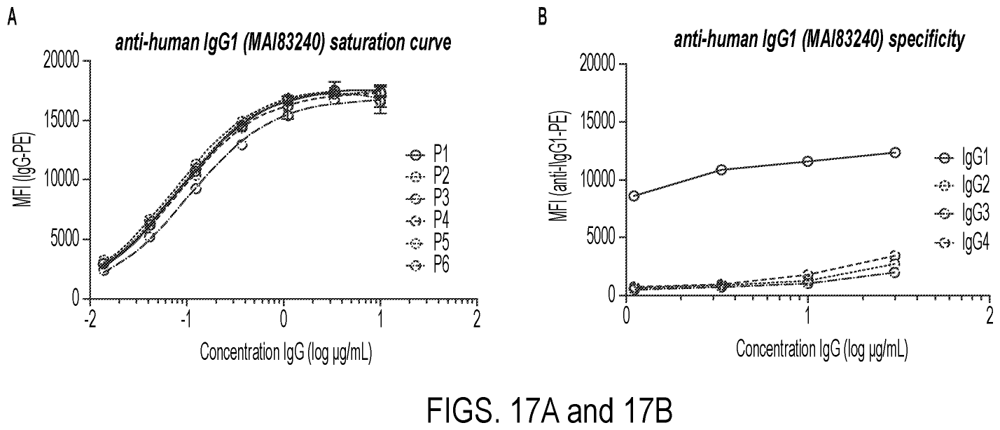

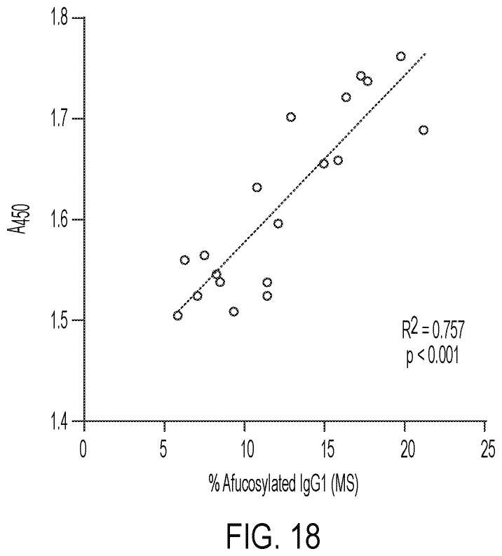

The level of afucosylated IgG1 can be a robust prognostic marker for severe dengue virus infection. A high level in newly admitted patients predicts disease progression to life-threatening dengue hemorrhagic fever (DHF) or dengue shock syndrome (DSS). However, previous studies have largely relied on low-throughput mass spectrometry methods to characterize levels of afucosylated IgG in patients. To provide a rapid and inexpensive alternative that can be delivered at point-of-care, B7 was adapted to standard clinical assays, such as sandwich ELISA or Luminex, to quantify afucosylated IgG1 in patient samples. First, the specificity of the leading nanobody candidates was confirmed by immunoprecipitation of IgG from human serum or IgG-depleted serum, demonstrating no binding to other serum glycoproteins (FIG. 16A). Using serum or purified IgG samples from outpatients from a previously published cohort whose IgG Fc glycan profiles have been characterized by mass spectrometry, both immunoassays capturing human IgG1 (FIGS. 17A-B) were performed, using B7 as the detection reagent. Consistent with the studies of homogeneous IgG glycoforms, nanobody-based quantification of afucosylated IgG in both patient purified IgG and serum demonstrated robust correlation with mass spectrometry values (FIGS. 12A-B and FIG. 18) and using purified IgG or diluted serum had minimal impact on assay output (FIG. 12C). To demonstrate the use of B7 as a rapid clinical prognostic, the nanobody-based assay was performed to quantify afucosylated IgG1 in purified IgG samples collected from dengue-infected pediatric patients upon hospital admission. Using the levels of afucosylated IgG1 derived from the assay, patients who eventually developed the mildest form of disease, dengue fever (DF), from those who progressed to DHIF or DSS could be distinguished (FIG. 12D). Receiver operating characteristic (ROC) analysis of the assay output of both ELISA and Luminex confirmed the prognostic value of Fc glycoform-specific nanobodies in predicting severe dengue disease progression (FIG. 12E), comparable to values determined by mass spectroscopy of purified IgG.

IgG Fc glycosylation continues to emerge as a dynamic and critical determinant of Fc-FcγR-mediated effector functions. Although the Fc glycome has been extensively characterized in several disease contexts, its study has been limited by the complexity and expense of conventional methods. For this reason, screening large patient cohorts is currently infeasible without tremendous resources and time. Thus, there is a need for a cheap and rapid method to measure the abundance of Fc glycoforms. The unique structural properties of nanobodies were exploited to engineer high affinity probes that specifically bind to afucosylated and sialylated IgG glycoforms with minimal cross-reactivity to other glycoforms. To the present inventors' knowledge, these probes are first-in-class as molecules that exclusively bind certain protein glycoforms. In characterizing these probes, it was demonstrated that binding is dependent on both protein and glycan structure, and that the lead candidate for afucosylated IgG binding recognizes a similar epitope on IgG as FcγRs, indicating its use as a potential therapeutic to disrupt pathogenic Fc-FcγR interactions, such as those proposed in antibody-dependent enhancement of dengue virus infection.

Due to their high affinity and selectivity, these nanobodies can be adapted to standard biochemical assays to measure the abundance of Fc glycoforms in patient serum samples. B7 accurately reported levels of afucosylated IgG1 in serum from dengue-infected patients, and in turn, predicted whether those patients progressed to severe disease, proving the capability of this reagent as a rapid prognostic tool.

### Example 4

### Therapeutic Applications Based on Blocking I2G Fc-Fcγ Receptor Interactions

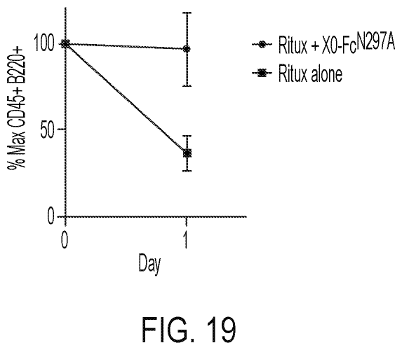

Afucosylated IgG has a proposed pathogenic function that enhances dengue virus and SARS-CoV-2 infection and causes more severe disease. In addition, an increase in afucosylated IgG is observed in some autoimmune diseases such as neonatal alloimmune thrombocytopenia (R. Kapur et al., Blood 123, 471-480 (2014)). These instances provide the basis for the development of therapeutics that specifically target IgG glycoforms. Based on structural studies that show the disclosed afucosylated IgG-specific nanobodies occlude the Fcγ receptor binding site on IgG, it was hypothesized that IgG Fc-Fcγ receptor interactions can be blocked with the nanobodies. To demonstrate the disclosed nanobodies as a therapeutic that specifically targets afucosylated IgG, the ability of the disclosed nanobodies in blocking antibody-mediated B cell depletion was tested (FIG. 19). This process is dependent on Fcγ receptor engagement by the administered antibody. Mice were injected intravenously with afucosylated rituximab (anti-CD20, 20 μg) with or without clone X0-Fc^{N297A} (200 μg). B cell depletion was measured after one day by flow cytometry (B cells counted as CD45⁺B220⁺). Addition of the nanobody-Fc fusion abrogated B cell depletion, demonstrating the efficacy of IgG glycoform-specific nanobodies as therapeutics. The results indicate that this disruption of IgG Fc-Fcγ receptor interactions is useful and clinically relevant in viral and autoimmune diseases where specific IgG glycoforms drive pathogenesis.

TABLE 6
Example amino acid sequences of representative nanobodies

| | | | SEQ |
|---|---|---|---|
| NANOBODY | REGION | SEQUENCE | ID NO |
| B7 | CDR1 | GGISRYKTM | 1 |
| | CDR2 | EFVAGITWGGSTYY | 2 |
| | CDR3 | SVDGGTYADPYHY | 3 |
| | Antigen- | QVQLQESGGGLVQAGGSLRLSCAASGGISRYKTMG | 4 |
| | binding | WYRQAPGKEREFVAGITWGGSTYYADSVKGRFTIS | |
| | fragment^{§} | RDNAKNTVYLQMNSLKPEDTAVYYCSVDGGTYAD | |
| | | PYHYYWGQGTQVTVSS | |
| mC11 | CDR1 | PGIYRYKTI | 5 |
| | CDR2 | SFVAAITWGGLTYR | 6 |
| | CDR3 | SVDGGTRAQPVHY | 7 |
| | Antigen- | QVQLQESGGGLVQAGGSLRLSCAASPGIYRYKTIA | 8 |
| | binding | WYRQAPGKERSFVAAITWGGLTYRADSVKGRFTV | |
| | fragment | SRDNAKNTVYLQMNSLKPEDTAVYYCSVDGGTRA | |
| | | QPVHYYWGQGTQVTVSS | |
| C11 | CDR1 | GYISRYHTM | 9 |
| | CDR2 | EFVAGITWGGSTYY | 10 |
| | CDR3 | AVDGGTYADPYHY | 11 |
| | Antigen- | QVQLQESGGGLVQAGGSLRLSCAASGYISRYHTMG | 12 |
| | binding | WYRQAPGKEREFVAGITWGGSTYYADSVKGRFTIS | |
| | fragment | RDNAKNTVYLQMNSLKPEDTAVYYCAVDGGTYAD | |
| | | PYHYYWGQGTQVTVSS | |
| E4 | CDR1 | PYISRYHTM | 13 |
| | CDR2 | EFVAAITWGGSTYY | 14 |
| | CDR3 | AVDGGTYADPYHY | 15 |
| | Antigen- | QVQLQESGGGLVQAGGSLRLSCAASPYISRYHTMG | 16 |
| | binding | WYRQAPGKEREFVAAITWGGSTYYADSVKGRFTIS | |
| | fragment | RDNAKNTVYLQMNSLKPEDTAVYYCAVDGGTYAD | |
| | | PYHYYWGQGTQVTVSS | |
| E2 | CDR1 | SYISRYHTM | 17 |
| | CDR2 | SFVAGITWGGLTYY | 18 |
| | CDR3 | AVDGGTRADPYHY | 19 |
| | Antigen- | QVQLQESGGGLVQAGGSLRLSCAASSYISRYHTMG | 20 |
| | binding | WYRQAPGKERSFVAGITWGGLTYYADSVKGRFTV | |
| | fragment | SRDNAKNTVYLQMNSLKPEDTAVYYCAVDGGTRA | |
| | | DPYHYYWGQGTQVTVSS | |
| X0 | CDR1 | PGISRYKTM | 21 |
| | CDR2 | SFVAAITWGGLTYY | 22 |
| | CDR3 | SVDGGTRADPYHY | 23 |
| | Antigen- | QVQLQESGGGLVQAGGSLRLSCAASPGISRYKTMG | 24 |
| | binding | WYRQAPGKERSFVAAITWGGLTYYADSVKGRFTV | |
| | fragment | SRDNAKNTVYLQMNSLKPEDTAVYYCSVDGGTRA | |
| | | DPYHYYWGQGTQVTVSS | |
| D3 | CDR1 | GNISADRYM | 25 |
| | CDR2 | EFVAAIGYGGTTYY | 26 |
| | CDR3 | AVVDGAHSRHRY | 27 |
| | Antigen- | QVQLQESGGGLVQAGGSLRLSCAASGNISADRYMG | 28 |
| | binding | WYRQAPGKEREFVAAIGYGGTTYYADSVKGRFTIS | |
| | fragment | RDNAKNTVYLQMNSLKPEDTAVYYCAVVDGAHSR | |
| | | HRYWGQGTQVTVSS | |
| C5 | CDR1 | GTISYGYVM | 29 |
| | CDR2 | ELVAGINRGSSTYY | 30 |
| | CDR3 | AASGDWYDWRSRYFLY | 31 |
| | Antigen- | QVQLQESGGGLVQAGGSLRLSCAASGTISYGYVMG | 32 |
| | binding | WYRQAPGKERELVAGINRGSSTYYADSVKGRFTIS | |
| | fragment | RDNAKNTVYLQMNSLKPEDTAVYYCAASGDWYD | |
| | | WRSRYFLYWGQGTQVTVSS | |
| H9 | CDR1 | GSISPLYNM | 33 |
| | CDR2 | EFVAGINSGSTTYY | 34 |
| | CDR3 | AAYTDGYEGLDY | 35 |
| | Antigen- | QVQLQESGGGLVQAGGSLRLSCAASGSISPLYNMG | 36 |
| | binding | WYRQAPGKEREFVAGINSGSTTYYADSVKGRFTISR | |
| | fragment | DNAKNTVYLQMNSLKPEDTAVYYCAAYTDGYEGL | |
| | | DYWGQGTQVTVSS | |

^{§}CDR1, CDR2, and CDR3 sequences are bolded and underlined.

TABLE 7
Example nucleic acid sequences of representative nanobodies

| | | SEQ |
|---|---|---|
| NANOBODY | SEQUENCE | ID NO |
| B7 | CAAGTACAACTGCAAGAGTCTGGAGGTGGACTTGTCCAGGCAG | 37 |
| | GCGGCTCGCTTCGTCTTTCTTGCGCTGCTAGTGGCGGGATCAGC | |
| | CGCTATAAAACAATGGGATGGTATCGTCAAGCGCCAGGCAAAG | |
| | AACGTGAATTTGTAGCTGGAATTACCTGGGGGGGATCTACATAT | |
| | TACGCTGACTCTGTCAAAGGCCGTTTCACTATCAGCCGTGACAA | |
| | CGCAAAAAATACCGTATATTTGCAAATGAATTCACTGAAACCCG | |
| | AAGACACAGCGGTGTATTATTGCTCCGTTGACGGGGGGACCTAC | |
| | GCTGACCCATACCATTACTACTGGGGGCAAGGGACCCAGGTAA | |
| | CAGTGTCCTCC | |
| mC11 | CAGGTCCAGTTACAAGAGTCAGGCGGCGGCTTGGTTCAAGCCG | 38 |
| | GCGGCAGTCTGCGTTTATCGTGTGCCGCATCCCCTGGGATTTAC | |
| | CGCTATAAAACCATCGCCTGGTATCGTCAGGCGCCTGGGAAAGA | |
| | ACGCAGCTTTGTTGCTGCAATCACATGGGGAGGGTTAACGTACC | |
| | GCGCAGATTCGGTTAAGGGGCGTTTTACCGTGTCCCGCGACAAT | |
| | GCAAAAAACACGGTATATCTTCAGATGAACTCGTTGAAACCAG | |
| | AAGACACAGCTGTTTACTACTGCTCGGTCGATGGTGGGACACGC | |
| | GCCCAGCCTGTGCATTACTACTGGGGCCAGGGTACGCAGGTTAC | |
| | AGTGTCGTCT | |
| C11 | CAGGTCCAGTTACAGGAGTCTGGGGGCGGCTTAGTCCAGGCCG | 39 |
| | GAGGGAGCTTGCGCTTGTCTTGTGCAGCTTCGGGCTATATTTCA | |
| | CGCTATCACACAATGGGATGGTATCGCCAAGCACCTGGAAAAG | |
| | AACGTGAATTTGTCGCTGGGATCACCTGGGGTGGATCTACCTAT | |
| | TATGCTGACAGTGTCAAGGGGCGCTTCACGATCTCGCGCGACAA | |
| | CGCAAAAAACACGGTTTACCTGCAAATGAACAGTCTTAAACCA | |
| | GAGGATACAGCCGTATATTACTGTGCAGTGGACGGAGGTACTTA | |
| | TGCTGACCCTTACCATTACTATTGGGGACAAGGAACCCAGGTAA | |
| | CTGTATCCAGC | |
| E4 | CAAGTGCAGCTTCAAGAGTCGGGCGGAGGTTTAGTACAAGCAG | 40 |
| | GGGGCTCGCTGCGCCTTTCATGTGCGGCAAGTCCCTACATTAGC | |
| | CGCTATCACACGATGGGATGGTATCGCCAAGCGCCAGGCAAAG | |
| | AACGCGAGTTCGTTGCAGCCATTACCTGGGGAGGCAGCACCTAC | |
| | TATGCTGATAGCGTAAAGGGCCGTTTCACGATCTCCCGTGATAA | |
| | CGCCAAAAACACGGTGTATTTGCAGATGAATTCTCTTAAACCGG | |
| | AGGATACTGCTGTATATTACTGCGCCGTGGACGGGGGAACGTAT | |
| | GCCGACCCCTATCACTATTATTGGGGACAAGGTACGCAAGTTAC | |
| | TGTTTCTAGC | |
| E2 | CAGGTACAATTGCAAGAGTCTGGAGGCGGCCTGGTCCAGGCAG | 41 |
| | GAGGTAGTTTACGCTTATCTTGTGCCGCGTCGTCCTACATTAGCC | |
| | GTTATCACACAATGGGATGGTACCGTCAAGCACCAGGGAAAGA | |
| | GCGTTCCTTTGTTGCTGGCATCACCTGGGGTGGCTTAACTTACTA | |
| | TGCAGATAGTGTCAAGGGGCGTTTCACGGTAAGTCGTGACAATG | |
| | CTAAGAACACTGTTTACTTACAAATGAACTCCCTTAAACCAGAA | |
| | GACACCGCCGTTTATTACTGCGCGGTGGACGGCGGCACCCGTGC | |
| | CGATCCTTACCATTATTACTGGGGGCAGGGGACACAAGTAACGG | |
| | TAAGTAGT | |
| X0 | CAAGTACAACTGCAAGAGTCTGGAGGTGGACTTGTCCAGGCGG | 42 |
| | GTGGCTCACTTCGCCTTTCATGTGCCGCTTCACCCGGGATCTCGC | |
| | GCTATAAGACAATGGGCTGGTACCGCCAAGCACCTGGAAAGGA | |
| | ACGTTCCTTCGTTGCCGCAATCACCTGGGGAGGTTTGACCTATT | |
| | ATGCCGATTCTGTTAAAGGGCGCTTCACAGTGTCGCGTGATAAC | |
| | GCAAAAAATACAGTGTATTTGCAGATGAACAGTTTGAAGCCTGA | |
| | AGACACGGCGGTTTACTATTGCAGTGTGGACGGTGGTACCCGTG | |
| | CCGATCCGTATCACTACTACTGGGGGCAAGGGACCCAGGTAAC | |
| | AGTGTCCTCC | |
| D3 | CAGGTTCAACTTCAAGAATCGGGAGGAGGACTGGTCCAAGCGG | 43 |
| | GAGGCAGCTTACGCCTTAGTTGTGCTGCCTCTGGAAATATCTCG | |
| | GCTGACCGCTACATGGGTTGGTACCGCCAGGCCCCTGGGAAAG | |
| | AGCGTGAGTTCGTGGCTGCAATCGGATACGGCGGAACCACTTAT | |
| | TATGCTGACAGTGTTAAGGGACGTTTCACTATCTCGCGCGATAA | |
| | TGCTAAGAATACAGTGTACCTTCAAATGAATTCTCTTAAACCAG | |
| | AGGACACCGCTGTTTATTACTGTGCTGTTGTGGACGGGGCGCAT | |
| | TCACGTCATCGTTACTGGGGACAAGGTACGCAGGTTACCGTAAG | |
| | TAGC | |
| C5 | CAGGTCCAGTTACAAGAATCAGGCGGCGGACTGGTCCAGGCTG | 44 |
| | GAGGCTCCCTTCGTTTAAGCTGCGCCGCTTCAGGAACGATTTCA | |
| | TACGGGTACGTCATGGGCTGGTACCGCCAAGCACCTGGCAAAG | |
| | AGCGCGAGCTTGTCGCGGGTATCAATCGCGGATCTTCGACGTAT | |
| | TATGCCGACAGCGTCAAAGGGCGTTTCACTATCTCCCGCGACAA | |
| | CGCGAAGAATACCGTCTACTTGCAAATGAACTCCCTGAAACCGG | |
| | AAGACACAGCCGTTTATTATTGTGCGGCAAGCGGGGACTGGTAT | |
| | GACTGGCGCAGCCGTTATTTCCTTTATTGGGGACAAGGTACTCA | |
| | GGTCACAGTTTCAAGC | |
| H9 | CAAGTGCAACTGCAGGAAAGCGGCGGCGGCCTGGTGCAGGCGG | 45 |
| | GCGGCAGCCTGCGCCTGAGCTGCGCGGCGAGCGGCTCTATTTCT | |
| | CCGCTGTACAACATGGGCTGGTATCGCCAGGCGCCGGGCAAAG | |
| | AACGCGAATTTGTTGCCGGTATTAATTCTGGTAGTACTACCTATT | |
| | ATGCGGATAGCGTGAAAGGCCGCTTTACCATTAGCCGCGATAAC | |
| | GCGAAAAACACCGTGTATCTGCAGATGAACAGCCTGAAACCGG | |
| | AAGATACCGCGGTGTATTATTGCGCGGCTTACACTGACGGTTAC | |
| | GAAGGTCTTGACTATTGGGGCCAGGGCACCCAGGTGACCGTGA | |
| | GCAGC | |

TABLE 8
Example sequences of representative nanobody fusion proteins

| NANOBODY | | SEQ |
|---|---|---|
| FUSION | SEQUENCE | ID NO |
| B7- | QVQLQESGGGLVQAGGSLRLSCAASGGISRYKTMGWYRQAPG | 48 |
| EndoS | KEREFVAGITWGGSTYYADSVKGRFTISRDNAKNTVYLQMNSL | |
| (glycosidase) | KPEDTAVYYCSVDGGTYADPYHYYWGQGTQVTVSSGGGGSG | |
| Nanobody | GGGSPLYGGYFRTWHDKTSDPTEKDKVNSMGELPKEVDLAFI | |
| is | FHDWTKDYSLFWKELATKHVPKLNKQGTRVIRTIPWRFLAGG | |
| underlined; | DNSGIAEDTSKYPNTPEGNKALAKAIVDEYVYKYNLDGLDVDV | |
| linker is | EHDSIPKVDKKEDTAGVERSIQVFEEIGKLIGPKGVDKSRLFIMD | |
| bolded | STYMADKNPLIERGAPYINLLLVQVYGSQGEKGGWEPVSNRPE | |
| | KTMEERWQGYSKYIRPEQYMIGFSFYEENAQEGNLWYDINSRK | |
| | DEDKANGINTDITGTRAERYARWQPKTGGVKGGIFSYAIDRDG | |
| | VAHQPKKYAKQKEFKDATDNIFHSDYSVSKALKTVM | |
| B7- | CAAGTACAACTGCAAGAGTCTGGAGGTGGACTTGTCCAGGC | 49 |
| EndoS | AGGCGGCTCGCTTCGTCTTTCTTGCGCTGCTAGTGGCGGGAT | |
| (glycosidase) | CAGCCGCTATAAAACAATGGGATGGTATCGTCAAGCGCCAG | |
| | GCAAAGAACGTGAATTTGTAGCTGGAATTACCTGGGGGGGA | |
| | TCTACATATTACGCTGACTCTGTCAAAGGCCGTTTCACTATC | |
| | AGCCGTGACAACGCAAAAAATACCGTATATTTGCAAATGAA | |
| | TTCACTGAAACCCGAAGACACAGCGGTGTATTATTGCTCCGT | |
| | TGACGGGGGGACCTACGCTGACCCATACCATTACTACTGGG | |
| | GGCAAGGGACCCAGGTAACAGTGTCCTCCGGAGGAGGAGGT | |
| | AGCGGCGGAGGCGGGTCTCCTCTCTACGGTGGTTACTTTAGA | |
| | ACTTGGCATGACAAAACATCAGATCCAACAGAAAAAGACAA | |
| | AGTTAACTCGATGGGAGAGCTTCCTAAAGAAGTAGATCTAG | |
| | CCTTTATTTTCCACGATTGGACAAAAGATTATAGCCTTTTTTG | |
| | GAAAGAATTGGCCACCAAACATGTGCCAAAGTTAAACAAGC | |
| | AAGGGACACGTGTCATTCGTACCATTCCATGGCGTTTCCTAG | |
| | CTGGGGGTGATAACAGTGGTATTGCAGAAGATACCAGTAAA | |
| | TACCCAAATACACCAGAGGGAAATAAAGCTTTAGCCAAAGC | |
| | TATTGTTGATGAATATGTTTATAAATACAACCTTGATGGCTT | |
| | AGATGTGGATGTTGAACATGATAGTATTCCAAAAGTTGACA | |
| | AAAAAGAAGATACAGCAGGCGTAGAACGCTCTATTCAAGTG | |
| | TTTGAAGAAATTGGGAAATTAATTGGACCAAAAGGTGTTGA | |
| | TAAATCGCGGTTATTTATTATGGATAGCACCTACATGGCTGA | |
| | TAAAAACCCATTGATTGAGCGAGGAGCTCCTTATATTAATTT | |
| | ATTACTGGTACAGGTCTATGGTTCACAAGGAGAGAAAGGTG | |
| | GTTGGGAGCCTGTTTCTAATCGACCTGAAAAAACAATGGAA | |
| | GAACGATGGCAAGGTTATAGCAAGTATATTCGTCCTGAACA | |
| | ATACATGATTGGTTTTTCTTTCTATGAGGAAAATGCTCAAGA | |
| | AGGGAATCTTTGGTATGATATTAATTCTCGCAAGGACGAGGA | |
| | CAAAGCAAATGGAATTAACACTGACATAACTGGAACGCGTG | |
| | CCGAACGGTATGCAAGGTGGCAACCTAAGACAGGTGGGGTT | |
| | AAGGGAGGTATCTTCTCCTACGCTATTGACCGAGATGGTGTA | |
| | GCTCATCAACCTAAAAAATATGCTAAACAGAAAGAGTTTAA | |
| | GGACGCAACTGATAACATCTTCCACTCAGATTATAGTGTCTC | |
| | CAAGGCATTAAAGACAGTTATG | |
| B7- | QVQLQESGGGLVQAGGSLRLSCAASGGISRYKTMGWYRQAPG | 50 |
| EndoS2 | KEREFVAGITWGGSTYYADSVKGRFTISRDNAKNTVYLQMNSL | |
| (glycosidase) | KPEDTAVYYCSVDGGTYADPYHYYWGQGTQVTVSSGGGGSG | |
| Nanobody | GGGSPLYAGYFRTWHDRASTGIDGKQQHPENTMAEVPKEVDI | |
| is | LFVFHDHTASDSPFWSELKDSYVHKLHQQGTALVQTIGVNELN | |
| underlined; | GRTGLSKDYPDTPEGNKALAAAIVKAFVTDRGVDGLDIDIEHEF | |
| linker is | TNKRTPEEDARALNVFKEIAQLIGKNGSDKSKLLIMDTTLSVEN | |
| bolded | NPIFKGIAEDLDYLLRQYYGSQGGEAEVDTINSDWNQYQNYID | |
| | ASQFMIGFSFFEESASKGNLWFDVNEYDPNNPEKGKDIEGTRA | |
| | KKYAEWQPSTGGLKAGIFSYAIDRDGVAHVPSTYKNRTSTNLQ | |
| | RHEVDNISHTDYTVSRKLKTLM | |
| B7- | CAAGTACAACTGCAAGAGTCTGGAGGTGGACTTGTCCAGGC | 51 |
| EndoS2 | AGGCGGCTCGCTTCGTCTTTCTTGCGCTGCTAGTGGCGGGAT | |
| (glycosidase) | CAGCCGCTATAAAACAATGGGATGGTATCGTCAAGCGCCAG | |
| | GCAAAGAACGTGAATTTGTAGCTGGAATTACCTGGGGGGGA | |
| | TCTACATATTACGCTGACTCTGTCAAAGGCCGTTTCACTATC | |
| | AGCCGTGACAACGCAAAAAATACCGTATATTTGCAAATGAA | |
| | TTCACTGAAACCCGAAGACACAGCGGTGTATTATTGCTCCGT | |
| | TGACGGGGGGACCTACGCTGACCCATACCATTACTACTGGG | |
| | GGCAAGGGACCCAGGTAACAGTGTCCTCCGGAGGAGGAGGT | |
| | AGCGGCGGAGGCGGGTCTccactatatgctggttattttaggacatggcatgatcgtg | |
| | cttcaacaggaatagatggtaaacagcaacatccagaaaatactatggctgaggtcccaaaagaagt | |
| | tgatatcttatttgtttttcatgaccatacagcttcagatagtccattttggtctgaattaaaggacagtt | |
| | atgtccataaattacatcaacagggaacggcacttgttcagacaattggtgttaacgaattaaatggacgt | |
| | acaggtttatctaaagattatcctgatactcctgaggggaacaaagctttagcagcagccattgtcaagg | |
| | catttgtaactgatcgtggtgtcgatggactagatattgatattgagcacgaatttacgaacaaaagaac | |
| | acctgaagaagatgctcgtgctctaaatgtttttaaagagattgcgcagttaataggtaaaaatggtagt | |
| | gataaatctaaattgctcatcatggacactaccctaagtgttgaaaataatccaatatttaaagggatag | |
| | cggaagatcttgattatcttcttagacaatattatggttcacaaggtggagaagctgaagtggatactat | |
| | aaactctgattggaaccaatatcagaattatattgatgctagccagttcatgattggattctccttttttg | |
| | aagaatctgcgtccaaagggaatttatggtttgatgttaacgaatacgaccctaacaatcctgaaaaaggg | |
| | aaagatattgaaggaacacgtgctaaaaaatatgcagagtggcaacctagtacaggtggtttaaaag | |
| | caggtatattctcttatgctattgatcgtgatggagtggctcatgttccttcaacatataaaaataggact | |
| | agtacaaatttacaacggcatgaagtcgataatatctcacatactgactacaccgtatctcgaaaattaaa | |
| | aacattgatg | |
| B7- | QVQLQESGGGLVQAGGSLRLSCAASGGISRYKTMGWYRQAPG | 52 |
| EndoS | KEREFVAGITWGGSTYYADSVKGRFTISRDNAKNTVYLQMNSL | |
| (full-length) | KPEDTAVYYCSVDGGTYADPYHYYWGQGTQVTVSSGGGGSG | |
| Nanobody | GGGSGGGGSMEEKTVQVQKGLPSIDSLHYLSENSKKEFKEELS | |
| is | KAGQESQKVKEILAKAQQADKQAQELAKMKIPEKIPMKPLHGP | |
| underlined; | LYGGYFRTWHDKTSDPTEKDKVNSMGELPKEVDLAFIFHDWT | |
| linker is | KDYSLFWKELATKHVPKLNKQGTRVIRTIPWRFLAGGDNSGIA | |
| bolded | EDTSKYPNTPEGNKALAKAIVDEYVYKYNLDGLDVDVEHDSIP | |
| | KVDKKEDTAGVERSIQVFEEIGKLIGPKGVDKSRLFIMDSTYMA | |
| | DKNPLIERGAPYINLLLVQVYGSQGEKGGWEPVSNRPEKTMEE | |
| | RWQGYSKYIRPEQYMIGFSFYEENAQEGNLWYDINSRKDEDKA | |
| | NGINTDITGTRAERYARWQPKTGGVKGGIFSYAIDRDGVAHQP | |
| | KKYAKQKEFKDATDNIFHSDYSVSKALKTVMLKDKSYDLIDEK | |
| | DFPDKALREAVMAQVGTRKGDLERFNGTLRLDNPAIQSLEGLN | |
| | KFKKLAQLDLIGLSRITKLDRSVLPANMKPGKDTLETVLETYKK | |
| | DNKEEPATIPPVSLKVSGLTGLKELDLSGFDRETLAGLDAATLT | |
| | SLEKVDISGNKLDLAPGTENRQIFDTMLSTISNHVGSNEQTVKF | |
| | DKQKPTGHYPDTYGKTSLRLPVANEKVDLQSQLLFGTVTNQGT | |
| | LINSEADYKAYQNHKIAGRSFVDSNYHYNNFKVSYENYTVKVT | |
| | DSTLGTTTDKTLATDKEETYKVDFFSPADKTKAVHTAKVIVGD | |
| | EKTMMVNLAEGATVIGGSADPVNARKVFDGQLGSETDNISLG | |
| | WDSKQSIIFKLKEDGLIKHWRFFNDSARNPETTNKPIQEASLQIF | |
| | NIKDYNLDNLLENPNKFDDEKYWITVDTYSAQGERATAFSNTL | |
| | NNITSKYWRVVFDTKGDRYSSPVVPELQILGYPLPNADTIMKTV | |
| | TTAKELSQQKDKFSQKMLDELKIKEMALETSLNSKIFDVTAINA | |
| | NAGVLKDCIEKRQLLKK | |
| B7- | CAAGTACAACTGCAAGAGTCTGGAGGTGGACTTGTCCAGGC | 53 |
| EndoS | AGGCGGCTCGCTTCGTCTTTCTTGCGCTGCTAGTGGCGGGAT | |
| (full-length) | CAGCCGCTATAAAACAATGGGATGGTATCGTCAAGCGCCAG | |
| | GCAAAGAACGTGAATTTGTAGCTGGAATTACCTGGGGGGGA | |
| | TCTACATATTACGCTGACTCTGTCAAAGGCCGTTTCACTATC | |
| | AGCCGTGACAACGCAAAAAATACCGTATATTTGCAAATGAA | |
| | TTCACTGAAACCCGAAGACACAGCGGTGTATTATTGCTCCGT | |
| | TGACGGGGGGACCTACGCTGACCCATACCATTACTACTGGG | |
| | GGCAAGGGACCCAGGTAACAGTGTCCTCCGGAGGAGGAGGT | |
| | AGCGGAGGAGGAGGTAGCGGCGGAGGCGGGTCTATGGAGG | |
| | AGAAGACTGTTCAGGTTCAGAAAGGATTACCTTCTATCGATA | |
| | GCTTGCATTATCTGTCAGAGAATAGCAAAAAAGAATTTAAA | |
| | GAAGAACTCTCAAAAGCGGGGCAAGAATCTCAAAAGGTCAA | |
| | AGAGATATTAGCAAAAGCTCAGCAGGCAGATAAACAAGCTC | |
| | AAGAACTTGCCAAAATGAAAATTCCTGAGAAAATACCGATG | |
| | AAACCGTTACATGGTCCTCTCTACGGTGGTTACTTTAGAACT | |
| | TGGCATGACAAAACATCAGATCCAACAGAAAAAGACAAAGT | |
| | TAACTCGATGGGAGAGCTTCCTAAAGAAGTAGATCTAGCCTT | |
| | TATTTTCCACGATTGGACAAAAGATTATAGCCTTTTTTGGAA | |
| | AGAATTGGCCACCAAACATGTGCCAAAGTTAAACAAGCAAG | |
| | GGACACGTGTCATTCGTACCATTCCATGGCGTTTCCTAGCTG | |
| | GGGGTGATAACAGTGGTATTGCAGAAGATACCAGTAAATAC | |
| | CCAAATACACCAGAGGGAAATAAAGCTTTAGCCAAAGCTAT | |
| | TGTTGATGAATATGTTTATAAATACAACCTTGATGGCTTAGA | |
| | TGTGGATGTTGAACATGATAGTATTCCAAAAGTTGACAAAA | |
| | AAGAAGATACAGCAGGCGTAGAACGCTCTATTCAAGTGTTT | |
| | GAAGAAATTGGGAAATTAATTGGACCAAAAGGTGTTGATAA | |
| | ATCGCGGTTATTTATTATGGATAGCACCTACATGGCTGATAA | |
| | AAACCCATTGATTGAGCGAGGAGCTCCTTATATTAATTTATT | |
| | ACTGGTACAGGTCTATGGTTCACAAGGAGAGAAAGGTGGTT | |
| | GGGAGCCTGTTTCTAATCGACCTGAAAAAACAATGGAAGAA | |
| | CGATGGCAAGGTTATAGCAAGTATATTCGTCCTGAACAATAC | |
| | ATGATTGGTTTTTCTTTCTATGAGGAAAATGCTCAAGAAGGG | |
| | AATCTTTGGTATGATATTAATTCTCGCAAGGACGAGGACAAA | |
| | GCAAATGGAATTAACACTGACATAACTGGAACGCGTGCCGA | |
| | ACGGTATGCAAGGTGGCAACCTAAGACAGGTGGGGTTAAGG | |
| | GAGGTATCTTCTCCTACGCTATTGACCGAGATGGTGTAGCTC | |
| | ATCAACCTAAAAAATATGCTAAACAGAAAGAGTTTAAGGAC | |
| | GCAACTGATAACATCTTCCACTCAGATTATAGTGTCTCCAAG | |
| | GCATTAAAGACAGTTATGCTAAAAGATAAGTCGTATGATCTG | |
| | ATTGATGAGAAAGATTTCCCAGATAAGGCTTTGCGAGAAGC | |
| | TGTGATGGCGCAGGTTGGAACCAGAAAAGGTGATTTGGAAC | |
| | GTTTCAATGGCACATTACGATTGGATAATCCAGCGATTCAAA | |
| | GTTTAGAAGGTCTAAATAAATTTAAAAAATTAGCTCAATTAG | |
| | TTTTACCCGCTAATATGAAGCCAGGCAAAGATACCTTGGAAA | |
| | CAGTTCTTGAAACCTATAAAAAGGATAACAAAGAAGAACCT | |
| | GCTACTATCCCACCAGTATCTTTGAAGGTTTCTGGTTTAACTG | |
| | GTCTGAAAGAATTAGATTTGTCAGGTTTTGACCGTGAAACCT | |
| | TGGCTGGTCTTGATGCCGCTACTCTAACGTCTTTAGAAAAAG | |
| | TTGATATTTCTGGCAACAAACTTGATTTGGCTCCAGGAACAG | |
| | AAAATCGACAAATTTTTGATACTATGCTATCAACTATCAGCA | |
| | ATCATGTTGGAAGCAATGAACAAACAGTGAAATTTGACAAG | |
| | CAAAAACCAACTGGGCATTACCCAGATACCTATGGGAAAAC | |
| | TAGTCTGCGCTTACCAGTGGCAAATGAAAAAGTTGATTTGCA | |
| | AAGCCAGCTTTTGTTTGGGACTGTGACAAATCAAGGAACCCT | |
| | AATCAATAGCGAAGCAGACTATAAGGCTTACCAAAATCATA | |
| | AAATTGCTGGACGTAGCTTTGTTGATTCAAACTATCATTACA | |
| | ATAACTTTAAAGTTTCTTATGAGAACTATACCGTTAAAGTAA | |
| | CTGATTCCACATTGGGAACCACTACTGACAAAACGCTAGCA | |
| | ACTGATAAAGAAGAGACCTATAAGGTTGACTTCTTTAGCCCA | |
| | GCAGATAAGACAAAAGCTGTTCATACTGCTAAAGTGATTGTT | |
| | GGTGACGAAAAAACCATGATGGTTAATTTGGCAGAAGGCGC | |
| | AACAGTTATTGGAGGAAGTGCTGATCCTGTAAATGCAAGAA | |
| | AGGTATTTGATGGGCAACTGGGCAGTGAGACTGATAATATCT | |
| | CTTTAGGATGGGATTCTAAGCAAAGTATTATATTTAAATTGA | |
| | AAGAAGATGGATTAATAAAGCATTGGCGTTTCTTCAATGATT | |
| | CAGCCCGAAATCCTGAGACAACCAATAAACCTATTCAGGAA | |
| | GCAAGTCTACAAATTTTTAATATCAAAGATTATAATCTAGAT | |
| | AATTTGTTGGAAAATCCCAATAAATTTGATGATGAAAAATAT | |
| | TGGATTACTGTAGATACTTACAGTGCACAAGGAGAGAGAGC | |
| | TACTGCATTCAGTAATACATTAAATAATATTACTAGTAAATA | |
| | TTGGCGAGTTGTCTTTGATACTAAAGGAGATAGATATAGTTC | |
| | GCCAGTAGTCCCTGAACTCCAAATTTTAGGTTATCCGTTACC | |
| | TAACGCCGACACTATCATGAAAACAGTAACTACTGCTAAAG | |
| | AGTTATCTCAACAAAAAGATAAGTTTTCTCAAAAGATGCTTG | |
| | ATGAGTTAAAAATAAAAGAGATGGCTTTAGAAACTTCTTTGA | |
| | ACAGTAAGATTTTTGATGTAACTGCTATTAATGCTAATGCTG | |
| | GAGTTTTGAAAGATTGTATTGAGAAAAGGCAGCTGCTAAAA | |
| | AAA | |
| B7- | QVQLQESGGGLVQAGGSLRLSCAASGGISRYKTMGWYRQAPG | 54 |
| EndoS2 | KEREFVAGITWGGSTYYADSVKGRFTISRDNAKNTVYLQMNSL | |
| (full-length) | KPEDTAVYYCSVDGGTYADPYHYYWGQGTQVTVSSGGGGSG | |
| Nanobody | GGGSGGGGSMGKTDQQVGAKLVQEIREGKRGPLYAGYFRTW | |
| is | HDRASTGIDGKQQHPENTMAEVPKEVDILFVFHDHTASDSPFW | |
| underlined; | SELKDSYVHKLHQQGTALVQTIGVNELNGRTGLSKDYPDTPEG | |
| linker is | NKALAAAIVKAFVTDRGVDGLDIDIEHEFTNKRTPEEDARALN | |
| bolded | VFKEIAQLIGKNGSDKSKLLIMDTTLSVENNPIFKGIAEDLDYLL | |
| | RQYYGSQGGEAEVDTINSDWNQYQNYIDASQFMIGFSFFEESAS | |
| | KGNLWFDVNEYDPNNPEKGKDIEGTRAKKYAEWQPSTGGLKA | |
| | GIFSYAIDRDGVAHVPSTYKNRTSTNLQRHEVDNISHTDYTVSR | |
| | KLKTLMTEDKRYDVIDQKDIPDPALREQIIQQVGQYKGDLERY | |
| | NKTLVLTGDKIQNLKGLEKLSKLQKLELRQLSNVKEITPELLPES | |
| | MKKDAELVMVGMTGLEKLNLSGLNRQTLDGIDVNSITHLTSFD | |
| | ISHNSLDLSEKSEDRKLLMTLMEQVSNHQKITVKNTAFENQKP | |
| | KGYYPQTYDTKEGHYDVDNAEHDILTDFVFGTVTKRNTFIGDE | |
| | EAFAIYKEGAVDGRQYVSKDYTYEAFRKDYKGYKVHLTASNL | |
| | GETVTSKVTATTDETYLVDVSDGEKVVHHMKLNIGSGAIMME | |
| | NLAKGAKVIGTSGDFEQAKKIFDGEKSDRFFTWGQTNWIAFDL | |
| | GEINLAKEWRLFNAETNTEIKTDSSLNVAKGRLQILKDTTIDLE | |
| | KMDIKNRKEYLSNDENWTDVAQMDDAKAIFNSKLSNVLSRYW | |
| | RFCVDGGASSYYPQYTELQILGQRLSNDVANTLKDL | |
| B7- | CAAGTACAACTGCAAGAGTCTGGAGGTGGACTTGTCCAGGC | 55 |
| EndoS2 | AGGCGGCTCGCTTCGTCTTTCTTGCGCTGCTAGTGGCGGGAT | |
| (full-length) | CAGCCGCTATAAAACAATGGGATGGTATCGTCAAGCGCCAG | |
| | GCAAAGAACGTGAATTTGTAGCTGGAATTACCTGGGGGGGA | |
| | TCTACATATTACGCTGACTCTGTCAAAGGCCGTTTCACTATC | |
| | AGCCGTGACAACGCAAAAAATACCGTATATTTGCAAATGAA | |
| | TTCACTGAAACCCGAAGACACAGCGGTGTATTATTGCTCCGT | |
| | TGACGGGGGGACCTACGCTGACCCATACCATTACTACTGGG | |
| | GGCAAGGGACCCAGGTAACAGTGTCCTCCGGAGGAGGAGGT | |
| | AGCGGAGGAGGAGGTAGCGGCGGAGGCGGGTCTatgggaaagaca | |
| | gatcagcaggttggtgctaaattggtacaggaaatccgtgaaggaaaacgcggaccactatatgctg | |
| | gttattttaggacatggcatgatcgtgcttcaacaggaatagatggtaaacagcaacatccagaaaata | |
| | ctatggctgaggtcccaaaagaagttgatatcttatttgtttttcatgaccatacagcttcagatagtcca | |
| | ttttggtctgaattaaaggacagttatgtccataaattacatcaacagggaacggcacttgttcagacaat | |
| | tggtgttaacgaattaaatggacgtacaggtttatctaaagattatcctgatactcctgaggggaacaaa | |
| | gctttagcagcagccattgtcaaggcatttgtaactgatcgtggtgtcgatggactagatattgatattg | |
| | agcacgaatttacgaacaaaagaacacctgaagaagatgctcgtgctctaaatgtttttaaagagattg | |
| | cgcagttaataggtaaaaatggtagtgataaatctaaattgctcatcatggacactaccctaagtgttga | |
| | aaataatccaatatttaaagggatagcggaagatcttgattatcttcttagacaatattatggttcacaag | |
| | gtggagaagctgaagtggatactataaactctgattggaaccaatatcagaattatattgatgctagcc | |
| | agttcatgattggattctccttttttgaagaatctgcgtccaaagggaatttatggtttgatgttaacgaa | |
| | tacgaccctaacaatcctgaaaaagggaaagatattgaaggaacacgtgctaaaaaatatgcagagtg | |
| | gcaacctagtacaggtggtttaaaagcaggtatattctcttatgctattgatcgtgatggagtggctcat | |
| | gttccttcaacatataaaaataggactagtacaaatttacaacggcatgaagtcgataatatctcacata | |
| | ctgactacaccgtatctcgaaaattaaaaacattgatgaccgaagacaaacgctatgatgtcattgatc | |
| | aaaaagacattcctgacccagcattaagagaacaaatcattcaacaagttggacagtataaaggcgat | |
| | ttggaacgttataacaagacattggtgcttacaggagataagattcaaaatcttaaaggactagaaaaa | |
| | ttaagcaagttacaaaaattagagttgcgccagctatctaacgttaaagaaattactccagaacttttgc | |
| | cggaaagcatgaaaaaagatgctgagcttgttatggtaggcatgactggtttagaaaaactaaacctt | |
| | agtggtctaaatcgtcaaactttagacggtatagacgtgaatagtattacgcatttgacatcatttgatat | |
| | ttcacataatagtttggacttgtcggaaaagagtgaagaccgtaaactattaatgactttgatggagcag | |
| | gtttcaaatcatcaaaaaataacggtgaaaaatacggcttttgaaaatcaaaaaccgaaaggttattatc | |
| | ctcagacgtatgataccaaagaaggtcattatgatgttgataatgcagaacatgatattttaactgatttt | |
| | gtttttggaactgttactaaacgtaatacctttattggagacgaagaagcatttgctatctataaagaagg | |
| | agctgtcgatggtcgacaatatgtgtctaaagactatacttatgaagcttttcgtaaagactataaaggtt | |
| | acaaggttcatttaactgcttctaacctaggagaaacagttacttctaaggtaactgctactactgatgaa | |
| | acttacttagtagatgtttctgatggggaaaaagttgttcaccacatgaaactcaatataggatctggtg | |
| | ccatcatgatggaaaatctggcaaaaggggctaaagtgattggtacatctggggactttgagcaagc | |
| | aaagaagattttcgatggtgaaaagtcagatagattcttcacttggggacaaactaactggatagctttt | |
| | gatctaggagaaattaatcttgcgaaggaatggcgtttatttaatgcagagacaaatactgaaataaag | |
| | acagatagtagcttaaacgtggctaaaggacgtcttcagattttaaaagatacaactattgatttagaaa | |
| | aaatggacataaaaaatcgtaaagagtatctgtcgaatgatgaaaattggactgatgttgctcagatgg | |
| | atgatgcaaaagcgatatttaatagtaaattatccaatgttttatctcggtattggcggttttgtgtagat | |
| | ggtggagctagctcttattaccctcaatataccgaacttcaaatcctoggacaacgtttatcaaatgatgt | |
| | cgctaatacgctgaaggatctg | |
| mC11- | QVQLQESGGGLVQAGGSLRLSCAASPGISRYKTMGWYRQAPG | 56 |
| EndoS | KERSFVAAITWGGLTYYADSVKGRFTVSRDNAKNTVYLQMNS | |
| (glycosidase) | LKPEDTAVYYCSVDGGTRADPYHYYWGQGTQVTVSSGGGGS | |
| Nanobody | GGGGSPLYGGYFRTWHDKTSDPTEKDKVNSMGELPKEVDLAF | |
| is | IFHDWTKDYSLFWKELATKHVPKLNKQGTRVIRTIPWRFLAGG | |
| underlined; | DNSGIAEDTSKYPNTPEGNKALAKAIVDEYVYKYNLDGLDVDV | |
| linker is | EHDSIPKVDKKEDTAGVERSIQVFEEIGKLIGPKGVDKSRLFIMD | |
| bolded | STYMADKNPLIERGAPYINLLLVQVYGSQGEKGGWEPVSNRPE | |
| | KTMEERWQGYSKYIRPEQYMIGFSFYEENAQEGNLWYDINSRK | |
| | DEDKANGINTDITGTRAERYARWQPKTGGVKGGIFSYAIDRDG | |
| | VAHQPKKYAKQKEFKDATDNIFHSDYSVSKALKTVM | |
| mC11- | CAAGTACAACTGCAAGAGTCTGGAGGTGGACTTGTCCAGGC | 57 |
| EndoS | GGGTGGCTCACTTCGCCTTTCATGTGCCGCTTCACCCGGGAT | |
| (glycosidase) | CTCGCGCTATAAGACAATGGGCTGGTACCGCCAAGCACCTG | |
| | GAAAGGAACGTTCCTTCGTTGCCGCAATCACCTGGGGAGGTT | |
| | TGACCTATTATGCCGATTCTGTTAAAGGGCGCTTCACAGTGT | |
| | CGCGTGATAACGCAAAAAATACAGTGTATTTGCAGATGAAC | |
| | AGTTTGAAGCCTGAAGACACGGCGGTTTACTATTGCAGTGTG | |
| | GACGGTGGTACCCGTGCCGATCCGTATCACTACTACTGGGGG | |
| | CAAGGGACCCAGGTAACAGTGTCCTCCGGAGGAGGAGGTAG | |
| | CGGCGGAGGCGGGTCTCCTCTCTACGGTGGTTACTTTAGAAC | |
| | TTGGCATGACAAAACATCAGATCCAACAGAAAAAGACAAAG | |
| | TTAACTCGATGGGAGAGCTTCCTAAAGAAGTAGATCTAGCCT | |
| | TTATTTTCCACGATTGGACAAAAGATTATAGCCTTTTTTGGA | |
| | AAGAATTGGCCACCAAACATGTGCCAAAGTTAAACAAGCAA | |
| | GGGACACGTGTCATTCGTACCATTCCATGGCGTTTCCTAGCT | |
| | GGGGGTGATAACAGTGGTATTGCAGAAGATACCAGTAAATA | |
| | CCCAAATACACCAGAGGGAAATAAAGCTTTAGCCAAAGCTA | |
| | TTGTTGATGAATATGTTTATAAATACAACCTTGATGGCTTAG | |
| | ATGTGGATGTTGAACATGATAGTATTCCAAAAGTTGACAAA | |
| | AAAGAAGATACAGCAGGCGTAGAACGCTCTATTCAAGTGTT | |
| | TGAAGAAATTGGGAAATTAATTGGACCAAAAGGTGTTGATA | |
| | AATCGCGGTTATTTATTATGGATAGCACCTACATGGCTGATA | |
| | AAAACCCATTGATTGAGCGAGGAGCTCCTTATATTAATTTAT | |
| | TACTGGTACAGGTCTATGGTTCACAAGGAGAGAAAGGTGGT | |
| | TGGGAGCCTGTTTCTAATCGACCTGAAAAAACAATGGAAGA | |
| | ACGATGGCAAGGTTATAGCAAGTATATTCGTCCTGAACAATA | |
| | CATGATTGGTTTTTCTTTCTATGAGGAAAATGCTCAAGAAGG | |
| | GAATCTTTGGTATGATATTAATTCTCGCAAGGACGAGGACAA | |
| | AGCAAATGGAATTAACACTGACATAACTGGAACGCGTGCCG | |
| | AACGGTATGCAAGGTGGCAACCTAAGACAGGTGGGGTTAAG | |
| | GGAGGTATCTTCTCCTACGCTATTGACCGAGATGGTGTAGCT | |
| | CATCAACCTAAAAAATATGCTAAACAGAAAGAGTTTAAGGA | |
| | CGCAACTGATAACATCTTCCACTCAGATTATAGTGTCTCCAA | |
| | GGCATTAAAGACAGTTATG | |
| mC11- | QVQLQESGGGLVQAGGSLRLSCAASPGISRYKTMGWYRQAPG | 58 |
| EndoS2 | KERSFVAAITWGGLTYYADSVKGRFTVSRDNAKNTVYLQMNS | |
| (glycosidase) | LKPEDTAVYYCSVDGGTRADPYHYYWGQGTQVTVSSGGGGS | |
| Nanobody | GGGGSPLYAGYFRTWHDRASTGIDGKQQHPENTMAEVPKEVD | |
| is | ILFVFHDHTASDSPFWSELKDSYVHKLHQQGTALVQTIGVNEL | |
| underlined; | NGRTGLSKDYPDTPEGNKALAAAIVKAFVTDRGVDGLDIDIEH | |
| linker is | EFTNKRTPEEDARALNVFKEIAQLIGKNGSDKSKLLIMDTTLSV | |
| bolded | ENNPIFKGIAEDLDYLLRQYYGSQGGEAEVDTINSDWNQYQNY | |
| | IDASQFMIGFSFFEESASKGNLWFDVNEYDPNNPEKGKDIEGTR | |
| | AKKYAEWQPSTGGLKAGIFSYAIDRDGVAHVPSTYKNRTSTNL | |
| | QRHEVDNISHTDYTVSRKLKTLM | |
| mC11- | CAAGTACAACTGCAAGAGTCTGGAGGTGGACTTGTCCAGGC | 59 |
| EndoS2 | GGGTGGCTCACTTCGCCTTTCATGTGCCGCTTCACCCGGGAT | |
| (glycosidase) | CTCGCGCTATAAGACAATGGGCTGGTACCGCCAAGCACCTG | |
| | GAAAGGAACGTTCCTTCGTTGCCGCAATCACCTGGGGAGGTT | |
| | TGACCTATTATGCCGATTCTGTTAAAGGGCGCTTCACAGTGT | |
| | CGCGTGATAACGCAAAAAATACAGTGTATTTGCAGATGAAC | |
| | AGTTTGAAGCCTGAAGACACGGCGGTTTACTATTGCAGTGTG | |
| | GACGGTGGTACCCGTGCCGATCCGTATCACTACTACTGGGGG | |
| | CAAGGGACCCAGGTAACAGTGTCCTCCGGAGGAGGAGGTAG | |
| | CGGCGGAGGCGGGTCTccactatatgctggttattttaggacatggcatgatcgtgcttc | |
| | aacaggaatagatggtaaacagcaacatccagaaaatactatggctgaggtcccaaaagaagttgat | |
| | atcttatttgtttttcatgaccatacagcttcagatagtccattttggtctgaattaaaggacagttatgt | |
| | ccataaattacatcaacagggaacggcacttgttcagacaattggtgttaacgaattaaatggacgtacag | |
| | gtttatctaaagattatcctgatactcctgaggggaacaaagctttagcagcagccattgtcaaggcattt | |
| | gtaactgatcgtggtgtcgatggactagatattgatattgagcacgaatttacgaacaaaagaacacct | |
| | gaagaagatgctcgtgctctaaatgtttttaaagagattgcgcagttaataggtaaaaatggtagtgata | |
| | aatctaaattgctcatcatggacactaccctaagtgttgaaaataatccaatatttaaagggatagcgga | |
| | agatcttgattatcttcttagacaatattatggttcacaaggtggagaagctgaagtggatactataaact | |
| | ctgattggaaccaatatcagaattatattgatgctagccagttcatgattggattctccttttttgaagaa | |
| | tctgcgtccaaagggaatttatggtttgatgttaacgaatacgaccctaacaatcctgaaaaagggaaag | |
| | atattgaaggaacacgtgctaaaaaatatgcagagtggcaacctagtacaggtggtttaaaagcaggt | |
| | atattctcttatgctattgatcgtgatggagtggctcatgttccttcaacatataaaaataggactagtac | |
| | aaatttacaacggcatgaagtcgataatatctcacatactgactacaccgtatctcgaaaattaaaaacat | |
| | tgatg | |
| mC11- | QVQLQESGGGLVQAGGSLRLSCAASPGISRYKTMGWYRQAPG | 60 |
| EndoS | KERSFVAAITWGGLTYYADSVKGRFTVSRDNAKNTVYLQMNS | |
| (full-length) | LKPEDTAVYYCSVDGGTRADPYHYYWGQGTQVTVSSGGGGS | |
| Nanobody | GGGGSMEEKTVQVQKGLPSIDSLHYLSENSKKEFKEELSKAGQ | |
| is | ESQKVKEILAKAQQADKQAQELAKMKIPEKIPMKPLHGPLYGG | |
| underlined; | YFRTWHDKTSDPTEKDKVNSMGELPKEVDLAFIFHDWTKDYS | |
| linker is | LFWKELATKHVPKLNKQGTRVIRTIPWRFLAGGDNSGIAEDTS | |
| bolded | KYPNTPEGNKALAKAIVDEYVYKYNLDGLDVDVEHDSIPKVD | |
| | KKEDTAGVERSIQVFEEIGKLIGPKGVDKSRLFIMDSTYMADKN | |
| | PLIERGAPYINLLLVQVYGSQGEKGGWEPVSNRPEKTMEERWQ | |
| | GYSKYIRPEQYMIGFSFYEENAQEGNLWYDINSRKDEDKANGI | |
| | NTDITGTRAERYARWQPKTGGVKGGIFSYAIDRDGVAHQPKKY | |
| | AKQKEFKDATDNIFHSDYSVSKALKTVMLKDKSYDLIDEKDFP | |
| | DKALREAVMAQVGTRKGDLERFNGTLRLDNPAIQSLEGLNKFK | |
| | KLAQLDLIGLSRITKLDRSVLPANMKPGKDTLETVLETYKKDN | |
| | KEEPATIPPVSLKVSGLTGLKELDLSGFDRETLAGLDAATLTSLE | |
| | KVDISGNKLDLAPGTENRQIFDTMLSTISNHVGSNEQTVKFDKQ | |
| | KPTGHYPDTYGKTSLRLPVANEKVDLQSQLLFGTVTNQGTLINS | |
| | EADYKAYQNHKIAGRSFVDSNYHYNNFKVSYENYTVKVTDST | |
| | LGTTTDKTLATDKEETYKVDFFSPADKTKAVHTAKVIVGDEKT | |
| | MMVNLAEGATVIGGSADPVNARKVFDGQLGSETDNISLGWDS | |
| | KQSIIFKLKEDGLIKHWRFFNDSARNPETTNKPIQEASLQIFNIKD | |
| | YNLDNLLENPNKFDDEKYWITVDTYSAQGERATAFSNTLNNIT | |
| | SKYWRVVFDTKGDRYSSPVVPELQILGYPLPNADTIMKTVTTA | |
| | KELSQQKDKFSQKMLDELKIKEMALETSLNSKIFDVTAINANAG | |
| | VLKDCIEKRQLLKK | |
| mC11- | CAAGTACAACTGCAAGAGTCTGGAGGTGGACTTGTCCAGGC 61 | |
| EndoS | GGGTGGCTCACTTCGCCTTTCATGTGCCGCTTCACCCGGGAT | |
| (full-length) | CTCGCGCTATAAGACAATGGGCTGGTACCGCCAAGCACCTG | |
| | GAAAGGAACGTTCCTTCGTTGCCGCAATCACCTGGGGAGGTT | |
| | TGACCTATTATGCCGATTCTGTTAAAGGGCGCTTCACAGTGT | |
| | CGCGTGATAACGCAAAAAATACAGTGTATTTGCAGATGAAC | |
| | AGTTTGAAGCCTGAAGACACGGCGGTTTACTATTGCAGTGTG | |
| | GACGGTGGTACCCGTGCCGATCCGTATCACTACTACTGGGGG | |
| | CAAGGGACCCAGGTAACAGTGTCCTCCGGAGGAGGAGGTAG | |
| | CGGCGGAGGCGGGTCTATGGAGGAGAAGACTGTTCAGGTTC | |
| | AGAAAGGATTACCTTCTATCGATAGCTTGCATTATCTGTCAG | |
| | AGAATAGCAAAAAAGAATTTAAAGAAGAACTCTCAAAAGCG | |
| | GGGCAAGAATCTCAAAAGGTCAAAGAGATATTAGCAAAAGC | |
| | TCAGCAGGCAGATAAACAAGCTCAAGAACTTGCCAAAATGA | |
| | AAATTCCTGAGAAAATACCGATGAAACCGTTACATGGTCCTC | |
| | TCTACGGTGGTTACTTTAGAACTTGGCATGACAAAACATCAG | |
| | ATCCAACAGAAAAAGACAAAGTTAACTCGATGGGAGAGCTT | |
| | CCTAAAGAAGTAGATCTAGCCTTTATTTTCCACGATTGGACA | |
| | AAAGATTATAGCCTTTTTTGGAAAGAATTGGCCACCAAACAT | |
| | GTGCCAAAGTTAAACAAGCAAGGGACACGTGTCATTCGTAC | |
| | CATTCCATGGCGTTTCCTAGCTGGGGGTGATAACAGTGGTAT | |
| | TGCAGAAGATACCAGTAAATACCCAAATACACCAGAGGGAA | |
| | ATAAAGCTTTAGCCAAAGCTATTGTTGATGAATATGTTTATA | |
| | AATACAACCTTGATGGCTTAGATGTGGATGTTGAACATGATA | |
| | GTATTCCAAAAGTTGACAAAAAAGAAGATACAGCAGGCGTA | |
| | GAACGCTCTATTCAAGTGTTTGAAGAAATTGGGAAATTAATT | |
| | GGACCAAAAGGTGTTGATAAATCGCGGTTATTTATTATGGAT | |
| | AGCACCTACATGGCTGATAAAAACCCATTGATTGAGCGAGG | |
| | AGCTCCTTATATTAATTTATTACTGGTACAGGTCTATGGTTCA | |
| | CAAGGAGAGAAAGGTGGTTGGGAGCCTGTTTCTAATCGACC | |
| | TGAAAAAACAATGGAAGAACGATGGCAAGGTTATAGCAAGT | |
| | ATATTCGTCCTGAACAATACATGATTGGTTTTTCTTTCTATGA | |
| | GGAAAATGCTCAAGAAGGGAATCTTTGGTATGATATTAATTC | |
| | TCGCAAGGACGAGGACAAAGCAAATGGAATTAACACTGACA | |
| | TAACTGGAACGCGTGCCGAACGGTATGCAAGGTGGCAACCT | |
| | AAGACAGGTGGGGTTAAGGGAGGTATCTTCTCCTACGCTATT | |
| | GACCGAGATGGTGTAGCTCATCAACCTAAAAAATATGCTAA | |
| | ACAGAAAGAGTTTAAGGACGCAACTGATAACATCTTCCACT | |
| | CAGATTATAGTGTCTCCAAGGCATTAAAGACAGTTATGCTAA | |
| | AAGATAAGTCGTATGATCTGATTGATGAGAAAGATTTCCCAG | |
| | ATAAGGCTTTGCGAGAAGCTGTGATGGCGCAGGTTGGAACC | |
| | AGAAAAGGTGATTTGGAACGTTTCAATGGCACATTACGATTG | |
| | GATAATCCAGCGATTCAAAGTTTAGAAGGTCTAAATAAATTT | |
| | AAAAAATTAGCTCAATTAGACTTGATTGGCTTATCTCGCATT | |
| | ACAAAGCTCGACCGTTCTGTTTTACCCGCTAATATGAAGCCA | |
| | GGCAAAGATACCTTGGAAACAGTTCTTGAAACCTATAAAAA | |
| | GGATAACAAAGAAGAACCTGCTACTATCCCACCAGTATCTTT | |
| | GAAGGTTTCTGGTTTAACTGGTCTGAAAGAATTAGATTTGTC | |
| | AGGTTTTGACCGTGAAACCTTGGCTGGTCTTGATGCCGCTAC | |
| | TCTAACGTCTTTAGAAAAAGTTGATATTTCTGGCAACAAACT | |
| | TGATTTGGCTCCAGGAACAGAAAATCGACAAATTTTTGATAC | |
| | TATGCTATCAACTATCAGCAATCATGTTGGAAGCAATGAACA | |
| | AACAGTGAAATTTGACAAGCAAAAACCAACTGGGCATTACC | |
| | CAGATACCTATGGGAAAACTAGTCTGCGCTTACCAGTGGCA | |
| | AATGAAAAAGTTGATTTGCAAAGCCAGCTTTTGTTTGGGACT | |
| | GTGACAAATCAAGGAACCCTAATCAATAGCGAAGCAGACTA | |
| | TAAGGCTTACCAAAATCATAAAATTGCTGGACGTAGCTTTGT | |
| | TGATTCAAACTATCATTACAATAACTTTAAAGTTTCTTATGA | |
| | GAACTATACCGTTAAAGTAACTGATTCCACATTGGGAACCAC | |
| | TACTGACAAAACGCTAGCAACTGATAAAGAAGAGACCTATA | |
| | AGGTTGACTTCTTTAGCCCAGCAGATAAGACAAAAGCTGTTC | |
| | ATACTGCTAAAGTGATTGTTGGTGACGAAAAAACCATGATG | |
| | GTTAATTTGGCAGAAGGCGCAACAGTTATTGGAGGAAGTGC | |
| | TGATCCTGTAAATGCAAGAAAGGTATTTGATGGGCAACTGG | |
| | GCAGTGAGACTGATAATATCTCTTTAGGATGGGATTCTAAGC | |
| | AAAGTATTATATTTAAATTGAAAGAAGATGGATTAATAAAG | |
| | CATTGGCGTTTCTTCAATGATTCAGCCCGAAATCCTGAGACA | |
| | ACCAATAAACCTATTCAGGAAGCAAGTCTACAAATTTTTAAT | |
| | ATCAAAGATTATAATCTAGATAATTTGTTGGAAAATCCCAAT | |
| | AAATTTGATGATGAAAAATATTGGATTACTGTAGATACTTAC | |
| | AGTGCACAAGGAGAGAGAGCTACTGCATTCAGTAATACATT | |
| | AAATAATATTACTAGTAAATATTGGCGAGTTGTCTTTGATAC | |
| | TAAAGGAGATAGATATAGTTCGCCAGTAGTCCCTGAACTCCA | |
| | AATTTTAGGTTATCCGTTACCTAACGCCGACACTATCATGAA | |
| | AACAGTAACTACTGCTAAAGAGTTATCTCAACAAAAAGATA | |
| | AGTTTTCTCAAAAGATGCTTGATGAGTTAAAAATAAAAGAG | |
| | ATGGCTTTAGAAACTTCTTTGAACAGTAAGATTTTTGATGTA | |
| | ACTGCTATTAATGCTAATGCTGGAGTTTTGAAAGATTGTATT | |
| | GAGAAAAGGCAGCTGCTAAAAAAA | |
| mC11- | QVQLQESGGGLVQAGGSLRLSCAASPGISRYKTMGWYRQAPG | 62 |
| EndoS2 | KERSFVAAITWGGLTYYADSVKGRFTVSRDNAKNTVYLQMNS | |
| (full-length) | LKPEDTAVYYCSVDGGTRADPYHYYWGQGTQVTVSSGGGGS | |
| Nanobody | GGGGSMGKTDQQVGAKLVQEIREGKRGPLYAGYFRTWHDRA | |
| is | STGIDGKQQHPENTMAEVPKEVDILFVFHDHTASDSPFWSELKD | |
| underlined; | SYVHKLHQQGTALVQTIGVNELNGRTGLSKDYPDTPEGNKALA | |
| linker is | AAIVKAFVTDRGVDGLDIDIEHEFTNKRTPEEDARALNVFKEIA | |
| bolded | QLIGKNGSDKSKLLIMDTTLSVENNPIFKGIAEDLDYLLRQYYG | |
| | SQGGEAEVDTINSDWNQYQNYIDASQFMIGFSFFEESASKGNL | |
| | WFDVNEYDPNNPEKGKDIEGTRAKKYAEWQPSTGGLKAGIFSY | |
| | AIDRDGVAHVPSTYKNRTSTNLQRHEVDNISHTDYTVSRKLKT | |
| | LMTEDKRYDVIDQKDIPDPALREQIIQQVGQYKGDLERYNKTL | |
| | VLTGDKIQNLKGLEKLSKLQKLELRQLSNVKEITPELLPESMKK | |
| | DAELVMVGMTGLEKLNLSGLNRQTLDGIDVNSITHLTSFDISHN | |
| | SLDLSEKSEDRKLLMTLMEQVSNHQKITVKNTAFENQKPKGYY | |
| | PQTYDTKEGHYDVDNAEHDILTDFVFGTVTKRNTFIGDEEAFAI | |
| | YKEGAVDGRQYVSKDYTYEAFRKDYKGYKVHLTASNLGETVT | |
| | SKVTATTDETYLVDVSDGEKVVHHMKLNIGSGAIMMENLAKG | |
| | AKVIGTSGDFEQAKKIFDGEKSDRFFTWGQTNWIAFDLGEINLA | |
| | KEWRLFNAETNTEIKTDSSLNVAKGRLQILKDTTIDLEKMDIKN | |
| | RKEYLSNDENWTDVAQMDDAKAIFNSKLSNVLSRYWRFCVDG | |
| | GASSYYPQYTELQILGQRLSNDVANTLKDL | |
| mC11- | CAAGTACAACTGCAAGAGTCTGGAGGTGGACTTGTCCAGGC | 63 |
| EndoS2 | GGGTGGCTCACTTCGCCTTTCATGTGCCGCTTCACCCGGGAT | |
| (full-length) | CTCGCGCTATAAGACAATGGGCTGGTACCGCCAAGCACCTG | |
| | GAAAGGAACGTTCCTTCGTTGCCGCAATCACCTGGGGAGGTT | |
| | TGACCTATTATGCCGATTCTGTTAAAGGGCGCTTCACAGTGT | |
| | CGCGTGATAACGCAAAAAATACAGTGTATTTGCAGATGAAC | |
| | AGTTTGAAGCCTGAAGACACGGCGGTTTACTATTGCAGTGTG | |
| | GACGGTGGTACCCGTGCCGATCCGTATCACTACTACTGGGGG | |
| | CAAGGGACCCAGGTAACAGTGTCCTCCGGAGGAGGAGGTAG | |
| | CGGCGGAGGCGGGTCTatgggaaagacagatcagcaggttggtgctaaattggtaca | |
| | ggaaatccgtgaaggaaaacgcggaccactatatgctggttattttaggacatggcatgatcgtgcttc | |
| | aacaggaatagatggtaaacagcaacatccagaaaatactatggctgaggtcccaaaagaagttgat | |
| | atcttatttgtttttcatgaccatacagcttcagatagtccattttggtctgaattaaaggacagttatgt | |
| | ccataaattacatcaacagggaacggcacttgttcagacaattggtgttaacgaattaaatggacgtacag | |
| | gtttatctaaagattatcctgatactcctgaggggaacaaagctttagcagcagccattgtcaaggcattt | |
| | gtaactgatcgtggtgtcgatggactagatattgatattgagcacgaatttacgaacaaaagaacacct | |
| | gaagaagatgctcgtgctctaaatgtttttaaagagattgcgcagttaataggtaaaaatggtagtgata | |
| | aatctaaattgctcatcatggacactaccctaagtgttgaaaataatccaatatttaaagggatagcgga | |
| | agatcttgattatcttcttagacaatattatggttcacaaggtggagaagctgaagtggatactataaact | |
| | ctgattggaaccaatatcagaattatattgatgctagccagttcatgattggattctccttttttgaagaa | |
| | tctgcgtccaaagggaatttatggtttgatgttaacgaatacgaccctaacaatcctgaaaaagggaaag | |
| | atattgaaggaacacgtgctaaaaaatatgcagagtggcaacctagtacaggtggtttaaaagcaggt | |
| | atattctcttatgctattgatcgtgatggagtggctcatgttccttcaacatataaaaataggactagtac | |
| | aaatttacaacggcatgaagtcgataatatctcacatactgactacaccgtatctcgaaaattaaaaacat | |
| | tgatgaccgaagacaaacgctatgatgtcattgatcaaaaagacattcctgacccagcattaagagaa | |
| | caaatcattcaacaagttggacagtataaaggcgatttggaacgttataacaagacattggtgcttaca | |
| | ggagataagattcaaaatcttaaaggactagaaaaattaagcaagttacaaaaattagagttgcgcca | |
| | gctatctaacgttaaagaaattactccagaacttttgccggaaagcatgaaaaaagatgctgagcttgtt | |
| | atggtaggcatgactggtttagaaaaactaaaccttagtggtctaaatcgtcaaactttagacggtatag | |
| | acgtgaatagtattacgcatttgacatcatttgatatttcacataatagtttggacttgtcggaaaagagt | |
| | gaagaccgtaaactattaatgactttgatggagcaggtttcaaatcatcaaaaaataacggtgaaaaata | |
| | cggcttttgaaaatcaaaaaccgaaaggttattatcctcagacgtatgataccaaagaaggtcattatg | |
| | atgttgataatgcagaacatgatattttaactgattttgtttttggaactgttactaaacgtaataccttt | |
| | attggagacgaagaagcatttgctatctataaagaaggagctgtcgatggtcgacaatatgtgtctaaaga | |
| | ctatacttatgaagcttttcgtaaagactataaaggttacaaggttcatttaactgcttctaacctaggag | |
| | aaacagttacttctaaggtaactgctactactgatgaaacttacttagtagatgtttctgatggggaaaaa | |
| | gttgttcaccacatgaaactcaatataggatctggtgccatcatgatggaaaatctggcaaaaggggcta | |
| | aagtgattggtacatctggggactttgagcaagcaaagaagattttcgatggtgaaaagtcagataga | |
| | ttcttcacttggggacaaactaactggatagcttttgatctaggagaaattaatcttgcgaaggaatggc | |
| | gtttatttaatgcagagacaaatactgaaataaagacagatagtagcttaaacgtggctaaaggacgtc | |
| | ttcagattttaaaagatacaactattgatttagaaaaaatggacataaaaaatcgtaaagagtatctgtcg | |
| | aatgatgaaaattggactgatgttgctcagatggatgatgcaaaagcgatatttaatagtaaattatcca | |
| | atgttttatctcggtattggcggttttgtgtagatggtggagctagctcttattaccctcaatataccgaa | |
| | cttcaaatcctoggacaacgtttatcaaatgatgtcgctaatacgctgaaggatctg | |
| EndoS (full | MEEKTVQVQKGLPSIDSLHYLSENSKKEFKEELSKAGQESQKV | 64 |
| length) | KEILAKAQQADKQAQELAKMKIPEKIPMKPLHGPLYGGYFRTW | |
| | HDKTSDPTEKDKVNSMGELPKEVDLAFIFHDWTKDYSLFWKEL | |
| | ATKHVPKLNKQGTRVIRTIPWRFLAGGDNSGIAEDTSKYPNTPE | |
| | GNKALAKAIVDEYVYKYNLDGLDVDVEHDSIPKVDKKEDTAG | |
| | VERSIQVFEEIGKLIGPKGVDKSRLFIMDSTYMADKNPLIERGAP | |
| | YINLLLVQVYGSQGEKGGWEPVSNRPEKTMEERWQGYSKYIRP | |
| | EQYMIGFSFYEENAQEGNLWYDINSRKDEDKANGINTDITGTR | |
| | AERYARWQPKTGGVKGGIFSYAIDRDGVAHQPKKYAKQKEFK | |
| | DATDNIFHSDYSVSKALKTVMLKDKSYDLIDEKDFPDKALREA | |
| | VMAQVGTRKGDLERFNGTLRLDNPAIQSLEGLNKFKKLAQLDL | |
| | IGLSRITKLDRSVLPANMKPGKDTLETVLETYKKDNKEEPATIPP | |
| | VSLKVSGLTGLKELDLSGFDRETLAGLDAATLTSLEKVDISGNK | |
| | LDLAPGTENRQIFDTMLSTISNHVGSNEQTVKFDKQKPTGHYPD | |
| | TYGKTSLRLPVANEKVDLQSQLLFGTVTNQGTLINSEADYKAY | |
| | QNHKIAGRSFVDSNYHYNNFKVSYENYTVKVTDSTLGTTTDKT | |
| | LATDKEETYKVDFFSPADKTKAVHTAKVIVGDEKTMMVNLAE | |
| | GATVIGGSADPVNARKVFDGQLGSETDNISLGWDSKQSIIFKLK | |
| | EDGLIKHWRFFNDSARNPETTNKPIQEASLQIFNIKDYNLDNLLE | |
| | NPNKFDDEKYWITVDTYSAQGERATAFSNTLNNITSKYWRVVF | |
| | DTKGDRYSSPVVPELQILGYPLPNADTIMKTVTTAKELSQQKDK | |
| | FSQKMLDELKIKEMALETSLNSKIFDVTAINANAGVLKDCIEKR | |
| | QLLKK | |
| EndoS (full | ATGGAGGAGAAGACTGTTCAGGTTCAGAAAGGATTACCTTC | 65 |
| length) | TATCGATAGCTTGCATTATCTGTCAGAGAATAGCAAAAAAG | |
| | AATTTAAAGAAGAACTCTCAAAAGCGGGGCAAGAATCTCAA | |
| | AAGGTCAAAGAGATATTAGCAAAAGCTCAGCAGGCAGATAA | |
| | ACAAGCTCAAGAACTTGCCAAAATGAAAATTCCTGAGAAAA | |
| | TACCGATGAAACCGTTACATGGTCCTCTCTACGGTGGTTACT | |
| | TTAGAACTTGGCATGACAAAACATCAGATCCAACAGAAAAA | |
| | GACAAAGTTAACTCGATGGGAGAGCTTCCTAAAGAAGTAGA | |
| | TCTAGCCTTTATTTTCCACGATTGGACAAAAGATTATAGCCT | |
| | TTTTTGGAAAGAATTGGCCACCAAACATGTGCCAAAGTTAAA | |
| | CAAGCAAGGGACACGTGTCATTCGTACCATTCCATGGCGTTT | |
| | CCTAGCTGGGGGTGATAACAGTGGTATTGCAGAAGATACCA | |
| | GTAAATACCCAAATACACCAGAGGGAAATAAAGCTTTAGCC | |
| | AAAGCTATTGTTGATGAATATGTTTATAAATACAACCTTGAT | |
| | GGCTTAGATGTGGATGTTGAACATGATAGTATTCCAAAAGTT | |
| | GACAAAAAAGAAGATACAGCAGGCGTAGAACGCTCTATTCA | |
| | AGTGTTTGAAGAAATTGGGAAATTAATTGGACCAAAAGGTG | |
| | TTGATAAATCGCGGTTATTTATTATGGATAGCACCTACATGG | |
| | CTGATAAAAACCCATTGATTGAGCGAGGAGCTCCTTATATTA | |
| | ATTTATTACTGGTACAGGTCTATGGTTCACAAGGAGAGAAAG | |
| | GTGGTTGGGAGCCTGTTTCTAATCGACCTGAAAAAACAATGG | |
| | AAGAACGATGGCAAGGTTATAGCAAGTATATTCGTCCTGAA | |
| | CAATACATGATTGGTTTTTCTTTCTATGAGGAAAATGCTCAA | |
| | GAAGGGAATCTTTGGTATGATATTAATTCTCGCAAGGACGAG | |
| | GACAAAGCAAATGGAATTAACACTGACATAACTGGAACGCG | |
| | TGCCGAACGGTATGCAAGGTGGCAACCTAAGACAGGTGGGG | |
| | TTAAGGGAGGTATCTTCTCCTACGCTATTGACCGAGATGGTG | |
| | TAGCTCATCAACCTAAAAAATATGCTAAACAGAAAGAGTTT | |
| | AAGGACGCAACTGATAACATCTTCCACTCAGATTATAGTGTC | |
| | TCCAAGGCATTAAAGACAGTTATGCTAAAAGATAAGTCGTA | |
| | TGATCTGATTGATGAGAAAGATTTCCCAGATAAGGCTTTGCG | |
| | AGAAGCTGTGATGGCGCAGGTTGGAACCAGAAAAGGTGATT | |
| | TGGAACGTTTCAATGGCACATTACGATTGGATAATCCAGCGA | |
| | TTCAAAGTTTAGAAGGTCTAAATAAATTTAAAAAATTAGCTC | |
| | AATTAGACTTGATTGGCTTATCTCGCATTACAAAGCTCGACC | |
| | GTTCTGTTTTACCCGCTAATATGAAGCCAGGCAAAGATACCT | |
| | TGGAAACAGTTCTTGAAACCTATAAAAAGGATAACAAAGAA | |
| | GAACCTGCTACTATCCCACCAGTATCTTTGAAGGTTTCTGGT | |
| | TTAACTGGTCTGAAAGAATTAGATTTGTCAGGTTTTGACCGT | |
| | GAAACCTTGGCTGGTCTTGATGCCGCTACTCTAACGTCTTTA | |
| | GAAAAAGTTGATATTTCTGGCAACAAACTTGATTTGGCTCCA | |
| | GGAACAGAAAATCGACAAATTTTTGATACTATGCTATCAACT | |
| | ATCAGCAATCATGTTGGAAGCAATGAACAAACAGTGAAATT | |
| | TGACAAGCAAAAACCAACTGGGCATTACCCAGATACCTATG | |
| | GGAAAACTAGTCTGCGCTTACCAGTGGCAAATGAAAAAGTT | |
| | GATTTGCAAAGCCAGCTTTTGTTTGGGACTGTGACAAATCAA | |
| | GGAACCCTAATCAATAGCGAAGCAGACTATAAGGCTTACCA | |
| | AAATCATAAAATTGCTGGACGTAGCTTTGTTGATTCAAACTA | |
| | TCATTACAATAACTTTAAAGTTTCTTATGAGAACTATACCGT | |
| | TAAAGTAACTGATTCCACATTGGGAACCACTACTGACAAAA | |
| | CGCTAGCAACTGATAAAGAAGAGACCTATAAGGTTGACTTC | |
| | TTTAGCCCAGCAGATAAGACAAAAGCTGTTCATACTGCTAAA | |
| | GTGATTGTTGGTGACGAAAAAACCATGATGGTTAATTTGGCA | |
| | GAAGGCGCAACAGTTATTGGAGGAAGTGCTGATCCTGTAAA | |
| | TGCAAGAAAGGTATTTGATGGGCAACTGGGCAGTGAGACTG | |
| | ATAATATCTCTTTAGGATGGGATTCTAAGCAAAGTATTATAT | |
| | TTAAATTGAAAGAAGATGGATTAATAAAGCATTGGCGTTTCT | |
| | TCAATGATTCAGCCCGAAATCCTGAGACAACCAATAAACCT | |
| | ATTCAGGAAGCAAGTCTACAAATTTTTAATATCAAAGATTAT | |
| | AATCTAGATAATTTGTTGGAAAATCCCAATAAATTTGATGAT | |
| | GAAAAATATTGGATTACTGTAGATACTTACAGTGCACAAGG | |
| | AGAGAGAGCTACTGCATTCAGTAATACATTAAATAATATTAC | |
| | TAGTAAATATTGGCGAGTTGTCTTTGATACTAAAGGAGATAG | |
| | ATATAGTTCGCCAGTAGTCCCTGAACTCCAAATTTTAGGTTA | |
| | TCCGTTACCTAACGCCGACACTATCATGAAAACAGTAACTAC | |
| | TGCTAAAGAGTTATCTCAACAAAAAGATAAGTTTTCTCAAAA | |
| | GATGCTTGATGAGTTAAAAATAAAAGAGATGGCTTTAGAAA | |
| | CTTCTTTGAACAGTAAGATTTTTGATGTAACTGCTATTAATGC | |
| | TAATGCTGGAGTTTTGAAAGATTGTATTGAGAAAAGGCAGCT | |
| | GCTAAAAAAA | |
| EndoS | PLYGGYFRTWHDKTSDPTEKDKVNSMGELPKEVDLAFIFHDW | 66 |
| (truncated) | TKDYSLFWKELATKHVPKLNKQGTRVIRTIPWRFLAGGDNSGI | |
| | AEDTSKYPNTPEGNKALAKAIVDEYVYKYNLDGLDVDVEHDSI | |
| | PKVDKKEDTAGVERSIQVFEEIGKLIGPKGVDKSRLFIMDSTYM | |
| | ADKNPLIERGAPYINLLLVQVYGSQGEKGGWEPVSNRPEKTME | |
| | ERWQGYSKYIRPEQYMIGFSFYEENAQEGNLWYDINSRKDEDK | |
| | ANGINTDITGTRAERYARWQPKTGGVKGGIFSYAIDRDGVAHQ | |
| | PKKYAKQKEFKDATDNIFHSDYSVSKALKTVM | |
| EndoS | CCTCTCTACGGTGGTTACTTTAGAACTTGGCATGACAAAACA | 67 |
| (truncated) | TCAGATCCAACAGAAAAAGACAAAGTTAACTCGATGGGAGA | |
| | GCTTCCTAAAGAAGTAGATCTAGCCTTTATTTTCCACGATTG | |
| | GACAAAAGATTATAGCCTTTTTTGGAAAGAATTGGCCACCAA | |
| | ACATGTGCCAAAGTTAAACAAGCAAGGGACACGTGTCATTC | |
| | GTACCATTCCATGGCGTTTCCTAGCTGGGGGTGATAACAGTG | |
| | GTATTGCAGAAGATACCAGTAAATACCCAAATACACCAGAG | |
| | GGAAATAAAGCTTTAGCCAAAGCTATTGTTGATGAATATGTT | |
| | TATAAATACAACCTTGATGGCTTAGATGTGGATGTTGAACAT | |
| | GATAGTATTCCAAAAGTTGACAAAAAAGAAGATACAGCAGG | |
| | CGTAGAACGCTCTATTCAAGTGTTTGAAGAAATTGGGAAATT | |
| | AATTGGACCAAAAGGTGTTGATAAATCGCGGTTATTTATTAT | |
| | GGATAGCACCTACATGGCTGATAAAAACCCATTGATTGAGC | |
| | GAGGAGCTCCTTATATTAATTTATTACTGGTACAGGTCTATG | |
| | GTTCACAAGGAGAGAAAGGTGGTTGGGAGCCTGTTTCTAAT | |
| | CGACCTGAAAAAACAATGGAAGAACGATGGCAAGGTTATAG | |
| | CAAGTATATTCGTCCTGAACAATACATGATTGGTTTTTCTTTC | |
| | TATGAGGAAAATGCTCAAGAAGGGAATCTTTGGTATGATATT | |
| | AATTCTCGCAAGGACGAGGACAAAGCAAATGGAATTAACAC | |
| | TGACATAACTGGAACGCGTGCCGAACGGTATGCAAGGTGGC | |
| | AACCTAAGACAGGTGGGGTTAAGGGAGGTATCTTCTCCTAC | |
| | GCTATTGACCGAGATGGTGTAGCTCATCAACCTAAAAAATAT | |
| | GCTAAACAGAAAGAGTTTAAGGACGCAACTGATAACATCTT | |
| | CCACTCAGATTATAGTGTCTCCAAGGCATTAAAGACAGTTAT | |
| | G | |
| EndoS2 | MGKTDQQVGAKLVQEIREGKRGPLYAGYFRTWHDRASTGIDG | 68 |
| (full- | KQQHPENTMAEVPKEVDILFVFHDHTASDSPFWSELKDSYVHK | |
| length) | LHQQGTALVQTIGVNELNGRTGLSKDYPDTPEGNKALAAAIVK | |
| | AFVTDRGVDGLDIDIEHEFTNKRTPEEDARALNVFKEIAQLIGK | |
| | NGSDKSKLLIMDTTLSVENNPIFKGIAEDLDYLLRQYYGSQGGE | |
| | AEVDTINSDWNQYQNYIDASQFMIGFSFFEESASKGNLWFDVN | |
| | EYDPNNPEKGKDIEGTRAKKYAEWQPSTGGLKAGIFSYAIDRD | |
| | GVAHVPSTYKNRTSTNLQRHEVDNISHTDYTVSRKLKTLMTED | |
| | KRYDVIDQKDIPDPALREQIIQQVGQYKGDLERYNKTLVLTGD | |
| | KIQNLKGLEKLSKLQKLELRQLSNVKEITPELLPESMKKDAELV | |
| | MVGMTGLEKLNLSGLNRQTLDGIDVNSITHLTSFDISHNSLDLS | |
| | EKSEDRKLLMTLMEQVSNHQKITVKNTAFENQKPKGYYPQTY | |
| | DTKEGHYDVDNAEHDILTDFVFGTVTKRNTFIGDEEAFAIYKEG | |
| | AVDGRQYVSKDYTYEAFRKDYKGYKVHLTASNLGETVTSKVT | |
| | ATTDETYLVDVSDGEKVVHHMKLNIGSGAIMMENLAKGAKVI | |
| | GTSGDFEQAKKIFDGEKSDRFFTWGQTNWIAFDLGEINLAKEW | |
| | RLFNAETNTEIKTDSSLNVAKGRLQILKDTTIDLEKMDIKNRKE | |
| | YLSNDENWTDVAQMDDAKAIFNSKLSNVLSRYWRFCVDGGAS | |
| | SYYPQYTELQILGQRLSNDVANTLKDL | |
| EndoS2 | atgggaaagacagatcagcaggttggtgctaaattggtacaggaaatccgtgaaggaaaacgcgga | 69 |
| (full-length) | ccactatatgctggttattttaggacatggcatgatcgtgcttcaacaggaatagatggtaaacagcaa | |
| | catccagaaaatactatggctgaggtcccaaaagaagttgatatcttatttgtttttcatgaccatacagc | |
| | ttcagatagtccattttggtctgaattaaaggacagttatgtccataaattacatcaacagggaacggca | |
| | cttgttcagacaattggtgttaacgaattaaatggacgtacaggtttatctaaagattatcctgatactcc | |
| | tgaggggaacaaagctttagcagcagccattgtcaaggcatttgtaactgatcgtggtgtcgatggact | |
| | agatattgatattgagcacgaatttacgaacaaaagaacacctgaagaagatgctcgtgctctaaatgt | |
| | ttttaaagagattgcgcagttaataggtaaaaatggtagtgataaatctaaattgctcatcatggacacta | |
| | ccctaagtgttgaaaataatccaatatttaaagggatagcggaagatcttgattatcttcttagacaatat | |
| | tatggttcacaaggtggagaagctgaagtggatactataaactctgattggaaccaatatcagaattata | |
| | ttgatgctagccagttcatgattggattctccttttttgaagaatctgcgtccaaagggaatttatggttt | |
| | gatgttaacgaatacgaccctaacaatcctgaaaaagggaaagatattgaaggaacacgtgctaaaaaa | |
| | tatgcagagtggcaacctagtacaggtggtttaaaagcaggtatattctcttatgctattgatcgtgatg | |
| | gagtggctcatgttccttcaacatataaaaataggactagtacaaatttacaacggcatgaagtcgata | |
| | atatctcacatactgactacaccgtatctcgaaaattaaaaacattgatgaccgaagacaaacgctatg | |
| | atgtcattgatcaaaaagacattcctgacccagcattaagagaacaaatcattcaacaagttggacagt | |
| | ataaaggcgatttggaacgttataacaagacattggtgcttacaggagataagattcaaaatcttaaag | |
| | gactagaaaaattaagcaagttacaaaaattagagttgcgccagctatctaacgttaaagaaattactc | |
| | cagaacttttgccggaaagcatgaaaaaagatgctgagcttgttatggtaggcatgactggtttagaaa | |
| | aactaaaccttagtggtctaaatcgtcaaactttagacggtatagacgtgaatagtattacgcatttgac | |
| | atcatttgatatttcacataatagtttggacttgtcggaaaagagtgaagaccgtaaactattaatgactt | |
| | tgatggagcaggtttcaaatcatcaaaaaataacggtgaaaaatacggcttttgaaaatcaaaaaccga | |
| | aaggttattatcctcagacgtatgataccaaagaaggtcattatgatgttgataatgcagaacatgatatt | |
| | ttaactgattttgtttttggaactgttactaaacgtaatacctttattggagacgaagaagcatttgctat | |
| | ctataaagaaggagctgtcgatggtcgacaatatgtgtctaaagactatacttatgaagcttttcgtaaag | |
| | actataaaggttacaaggttcatttaactgcttctaacctaggagaaacagttacttctaaggtaactgct | |
| | actactgatgaaacttacttagtagatgtttctgatggggaaaaagttgttcaccacatgaaactcaatat | |
| | aggatctggtgccatcatgatggaaaatctggcaaaaggggctaaagtgattggtacatctggggactt | |
| | tgagcaagcaaagaagattttcgatggtgaaaagtcagatagattcttcacttggggacaaactaact | |
| | ggatagcttttgatctaggagaaattaatcttgcgaaggaatggcgtttatttaatgcagagacaaatac | |
| | tgaaataaagacagatagtagcttaaacgtggctaaaggacgtcttcagattttaaaagatacaactatt | |
| | gatttagaaaaaatggacataaaaaatcgtaaagagtatctgtcgaatgatgaaaattggactgatgtt | |
| | gctcagatggatgatgcaaaagcgatatttaatagtaaattatccaatgttttatctcggtattggcggtt | |
| | ttgtgtagatggtggagctagctcttattaccctcaatataccgaacttcaaatcctcggacaacgtttat | |
| | caaatgatgtcgctaatacgctgaaggatctg | |
| EndoS2 | PLYAGYFRTWHDRASTGIDGKQQHPENTMAEVPKEVDILFVFH | 70 |
| (truncated) | DHTASDSPFWSELKDSYVHKLHQQGTALVQTIGVNELNGRTGL | |
| | SKDYPDTPEGNKALAAAIVKAFVTDRGVDGLDIDIEHEFTNKRT | |
| | PEEDARALNVFKEIAQLIGKNGSDKSKLLIMDTTLSVENNPIFKG | |
| | IAEDLDYLLRQYYGSQGGEAEVDTINSDWNQYQNYIDASQFMI | |
| | GFSFFEESASKGNLWFDVNEYDPNNPEKGKDIEGTRAKKYAEW | |
| | QPSTGGLKAGIFSYAIDRDGVAHVPSTYKNRTSTNLQRHEVDNI | |
| | SHTDYTVSRKLKTLM | |
| EndoS2 | ccactatatgctggttattttaggacatggcatgatcgtgcttcaacaggaatagatggtaaacagcaa | 71 |
| (truncated) | catccagaaaatactatggctgaggtcccaaaagaagttgatatcttatttgtttttcatgaccatacagc | |
| | ttcagatagtccattttggtctgaattaaaggacagttatgtccataaattacatcaacagggaacggca | |
| | cttgttcagacaattggtgttaacgaattaaatggacgtacaggtttatctaaagattatcctgatactcct | |
| | gaggggaacaaagctttagcagcagccattgtcaaggcatttgtaactgatcgtggtgtcgatggact | |
| | agatattgatattgagcacgaatttacgaacaaaagaacacctgaagaagatgctcgtgctctaaatgt | |
| | ttttaaagagattgcgcagttaataggtaaaaatggtagtgataaatctaaattgctcatcatggacacta | |
| | ccctaagtgttgaaaataatccaatatttaaagggatagcggaagatcttgattatcttcttagacaatatt | |
| | atggttcacaaggtggagaagctgaagtggatactataaactctgattggaaccaatatcagaattata | |
| | ttgatgctagccagttcatgattggattctccttttttgaagaatctgcgtccaaagggaatttatggttt | |
| | gatgttaacgaatacgaccctaacaatcctgaaaaagggaaagatattgaaggaacacgtgctaaaaaa | |
| | tatgcagagtggcaacctagtacaggtggtttaaaagcaggtatattctcttatgctattgatcgtgatg | |
| | gagtggctcatgttccttcaacatataaaaataggactagtacaaatttacaacggcatgaagtcgata | |
| | atatctcacatactgactacaccgtatctcgaaaattaaaaacattgatg | |
| IdeS | MRKRCYSTSAVVLAAVTLFALSVDRGVIADSFSANQEIRYSEVT | 72 |
| | PYHVTSVWTKGVTPPAKFTQGEDVFHAPYVANQGWYDITKTF | |
| | NGKDDLLCGAATAGNMLHWWFDQNKEKIEAYLKKHPDKQKI | |
| | MFGDQELLDVRKVINTKGDQTNSELFNYFRDKAFPGLSARRIG | |
| | VMPDLVLDMFINGYYLNVYKTQTTDVNRTYQEKDRRGGIFDA | |
| | VFTRGDQSKLLTSRHDFKEKNLKEISDLIKKELTEGKALGLSHT | |
| | YANVRINHVINLWGADFDSNGNLKAIYVTDSDSNASIGMKKYF | |
| | VGVNSAGKVAISAKEIKEDNIGAQVLGLFTLSTGQDSWNQTN | |
| IdeS | ATGAGAAAAAGATGCTATTCAACTTCAGCTGTAGTATTGGCA | 73 |
| | GCAGTGACTTTATTTGCTCTATCGGTAGATCGTGGTGTTATA | |
| | GCAGATAGTTTTTCTGCTAATCAAGAGATTAGATATTCGGAA | |
| | GTAACACCTTATCATGTTACTTCCGTTTGGACCAAAGGAGTT | |
| | ACTCCTCCAGCAAAATTCACTCAAGGCGAAGATGTTTTTCAC | |
| | GCTCCTTATGTTGCTAACCAAGGATGGTATGATATTACCAAA | |
| | ACATTCAATGGAAAAGACGATCTTCTTTGCGGGGCTGCCACA | |
| | GCAGGGAATATGCTTCACTGGTGGTTCGATCAAAACAAAGA | |
| | AAAAATTGAAGCATATCTAAAAAAACACCCAGATAAACAAA | |
| | AAATCATGTTTGGTGATCAAGAATTATTGGATGTAAGAAAA | |
| | GTTATTAATACCAAAGGTGACCAAACAAATAGCGAGCTTTTT | |
| | AATTATTTCCGAGATAAAGCTTTCCCCGGTTTGTCAGCACGC | |
| | CGAATTGGAGTTATGCCTGATCTTGTTTTAGATATGTTTATCA | |
| | ATGGTTATTACTTAAATGTTTATAAGACACAGACTACTGATG | |
| | TCAATAGAACCTATCAAGAGAAAGATCGCCGAGGTGGTATT | |
| | TTTGACGCCGTATTTACAAGAGGTGATCAAAGTAAGCTATTG | |
| | ACAAGTCGTCATGATTTTAAAGAAAAAAATCTCAAAGAAAT | |
| | CAGTGATCTCATTAAGAAAGAGTTAACCGAAGGCAAGGCTC | |
| | TAGGCCTATCACACACCTACGCTAACGTACGCATCAACCATG | |
| | TTATAAACCTGTGGGGAGCTGACTTTGATTCTAACGGGAACC | |
| | TTAAAGCTATTTATGTAACAGACTCTGATAGTAATGCATCTA | |
| | TTGGTATGAAGAAATACTTTGTTGGTGTTAATTCCGCTGGAA | |
| | AAGTAGCTATTTCTGCTAAAGAAATAAAAGAAGATAATATT | |
| | GGTGCTCAAGTACTAGGGTTATTTACACTTTCAACAGGGCAA | |
| | GATAGTTGGAATCAGACCAATTAA | |

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. An isolated nanobody that binds specifically to an IgG Fe glycoform, comprising three complementarity determining regions (CDR1, CDR2, and CDR3), wherein:
(a) CDR1 comprises the amino acid sequence of SEQ ID NO: 1; CDR2 comprises the amino acid sequence of SEQ ID NO: 2; and CDR3 comprises the amino acid sequence of SEQ ID NO: 3;
(b) CDR1 comprises the amino acid sequence of SEQ ID NO: 5; CDR2 comprises the amino acid sequence of SEQ ID NO: 6; and CDR3 comprises the amino acid sequence of SEQ ID NO: 7;
(c) CDR1 comprises the amino acid sequence of SEQ ID NO: 9; CDR2 comprises the amino acid sequence of SEQ ID NO: 10; and CDR3 comprises the amino acid sequence of SEQ ID NO: 11;
(d) CDR1 comprises the amino acid sequence of SEQ ID NO: 13; CDR2 comprises the amino acid sequence of SEQ ID NO: 14; and CDR3 comprises the amino acid sequence of SEQ ID NO: 15;
(e) CDR1 comprises the amino acid sequence of SEQ ID NO: 17; CDR2 comprises the amino acid sequence of SEQ ID NO: 18; and CDR3 comprises the amino acid sequence of SEQ ID NO: 19;
(f) CDR1 comprises the amino acid sequence of SEQ ID NO: 21; CDR2 comprises the amino acid sequence of SEQ ID NO: 22; and CDR3 comprises the amino acid sequence of SEQ ID NO: 23;
(g) CDR1 comprises the amino acid sequence of SEQ ID NO: 25; CDR2 comprises the amino acid sequence of SEQ ID NO: 26; and CDR3 comprises the amino acid sequence of SEQ ID NO: 27;
(h) CDR1 comprises the amino acid sequence of SEQ ID NO: 29; CDR2 comprises the amino acid sequence of SEQ ID NO: 30; and CDR3 comprises the amino acid sequence of SEQ ID NO: 31; or
(i) CDR1 comprises the amino acid sequence of SEQ ID NO: 33; CDR2 comprises the amino acid sequence of SEQ ID NO: 34; and CDR3 comprises the amino acid sequence of SEQ ID NO: 35.

2. The nanobody or antigen-binding fragment thereof of claim 1, comprising an amino acid sequence having at least 80% sequence identity with the amino acid sequence of SEQ ID NO: 4, 8, 12, 16, 20, 24, 28, 32, or 36, or comprising the amino acid sequence of SEQ ID NO: 4, 8, 12, 16, 20, 24, 28, 32, or 36.

3. The nanobody or antigen-binding fragment thereof of any one of the preceding claims, wherein the nanobody or antigen-binding fragment thereof binds specifically to an IgG1 Fc glycoform.

4. The nanobody or antigen-binding fragment thereof of any one of the preceding claims, wherein the nanobody or antigen-binding fragment thereof binds specifically to an afucosylated IgG1 Fc glycoform.

5. The nanobody or antigen-binding fragment thereof of any one of the preceding claims, wherein the nanobody or antigen-binding fragment thereof binds specifically to an IgG1 Fc glycoform afucosylated at Asp297 (EU numbering).

6. The nanobody or antigen-binding fragment thereof of any one of claims 1 to 3, wherein the nanobody or antigen-binding fragment thereof binds specifically to a sialylated IgG1 Fc glycoform.

7. The nanobody or antigen-binding fragment thereof of any one of the preceding claims, wherein the nanobody or antigen-binding fragment thereof competes for binding to the IgG Fc glycoform against a Fcγ receptor IIIA (FcγRIIIA).

8. The nanobody or antigen-binding fragment thereof of any one of the preceding claims, wherein the IgG Fc glycoform is an IgG Fc glycoform of an anti-dengue virus (DENV) antibody or an anti-SARS-CoV-2 antibody.

9. The nanobody or antigen-binding fragment thereof of any one of the preceding claims, wherein two or more of the nanobody or antigen-binding fragment thereof are linked to each other directly or via a linker.

10. The nanobody or antigen-binding fragment thereof of claim 9, wherein the nanobody or antigen-binding fragment thereof oligomerizes as a tetramer.

11. The nanobody or antigen-binding fragment thereof of any one of the preceding claims, wherein the nanobody or antigen-binding fragment thereof is detectably labeled or conjugated to a toxin, a therapeutic agent, a polymer, a receptor, an enzyme, or a receptor ligand.

12. The nanobody or antigen-binding fragment thereof of claim 11, wherein the polymer is polyethylene glycol (PEG).

13. The nanobody or antigen-binding fragment thereof of any one of the preceding claims, wherein the nanobody or antigen-binding fragment thereof is biotinylated.

14. The nanobody or antigen-binding fragment thereof of any one of the preceding claims, wherein the nanobody or antigen-binding fragment thereof is a humanized nanobody.

15. An isolated antibody or antigen-binding fragment thereof that binds specifically to an IgG Fc glycoform, comprising three heavy chain complementarity determining regions (HCDR1, HCDR2, and HCDR3), wherein: (i) HCDR1 comprises the amino acid sequence of SEQ ID NO: 1; HCDR2 comprises the amino acid sequence of SEQ ID NO: 2; and HCDR3 comprises the amino acid sequence of SEQ ID NO: 3; (ii) HCDR1 comprises the amino acid sequence of SEQ ID NO: 5; HCDR2 comprises the amino acid sequence of SEQ ID NO: 6; and HCDR3 comprises the amino acid sequence of SEQ ID NO: 7; (iii) HCDR1 comprises the amino acid sequence of SEQ ID NO: 9; HCDR2 comprises the amino acid sequence of SEQ ID NO: 10; and HCDR3 comprises the amino acid sequence of SEQ ID NO: 11; (iv) HCDR1 comprises the amino acid sequence of SEQ ID NO: 13; HCDR2 comprises the amino acid sequence of SEQ ID NO: 14; and HCDR3 comprises the amino acid sequence of SEQ ID NO: 15; (v) HCDR1 comprises the amino acid sequence of SEQ ID NO: 17; HCDR2 comprises the amino acid sequence of SEQ ID NO: 18; and HCDR3 comprises the amino acid sequence of SEQ ID NO: 19; (vi) HCDR1 comprises the amino acid sequence of SEQ ID NO: 21; HCDR2 comprises the amino acid sequence of SEQ ID NO: 22; and HCDR3 comprises the amino acid sequence of SEQ ID NO: 23; (vii) HCDR1 comprises the amino acid sequence of SEQ ID NO: 25; HCDR2 comprises the amino acid sequence of SEQ ID NO: 26; and HCDR3 comprises the amino acid sequence of SEQ ID NO: 27; (viii) HCDR1 comprises the amino acid sequence of SEQ ID NO: 29; HCDR2 comprises the amino acid sequence of SEQ ID NO: 30; and HCDR3 comprises the amino acid sequence of SEQ ID NO: 31; or (ix) HCDR1 comprises the amino acid sequence of SEQ ID NO: 33; HCDR2 comprises the amino acid sequence of SEQ ID NO: 34; and HCDR3 comprises the amino acid sequence of SEQ ID NO: 35.

16. The antibody or antigen-binding fragment thereof of claim 15, comprising a heavy chain variable region (HCVR) that comprises an amino acid sequence having at least 80% sequence identity with the amino acid sequence of SEQ ID NO: 4, 8, 12, 16, 20, 24, 28, 32, or 36, or comprising the amino acid sequence of SEQ ID NO: 4, 8, 12, 16, 20, 24, 28, 32, or 36.

17. A polypeptide comprising at least one nanobody or antigen-binding fragment thereof of any one of claims 1 to 14 or the antibody or antigen-binding fragment thereof of any one of claims 15 to 16.

18. The polypeptide of claim 17, comprising two or more nanobodies or antigen-binding fragments thereof of any one of claims 1 to 14 linked to each other directly or via a linker.

19. The polypeptide of any one of claims 17 to 18, wherein the linker comprises a peptide linker or a disulfide bond.

20. The polypeptide of any one of claims 17 to 19, comprising (a) a first nanobody or antigen-binding fragment thereof and a second nanobody or antigen-binding fragment thereof according to any one of claims 1 to 14, wherein the first nanobody or antigen-binding fragment thereof and the second nanobody or antigen-binding fragment bind to different epitopes in the IgG Fc glycoform; or (b) a first nanobody or antigen-binding fragment thereof, a second nanobody or antigen-binding fragment thereof, and a third nanobody or antigen-binding fragment thereof according to any one of claims 1 to 14, wherein the first nanobody or antigen-binding fragment thereof, the second nanobody or antigen-binding fragment, and the third nanobody or antigen-binding fragment thereof bind to different epitopes in the IgG Fc glycoform.

21. The polypeptide of claim 17, wherein the polypeptide comprises the nanobody or antigen-binding fragment thereof of any one of claims 1 to 14 or the antibody or antigen-binding fragment thereof of any one of claims 15 to 16, linked to an endoglycosidase or proteinase directly or via a linker.

22. The polypeptide of claim 21, wherein the endoglycosidase or proteinase comprises EndoS, EndoS2, or IdeS from Streptococcus pyogene.

23. The polypeptide of any one of claims 21 to 22, wherein the endoglycosidase or proteinase comprises an amino acid sequence having at least 80% sequence identity with the amino acid sequence of SEQ ID NO: 64, 66, 68, 70, or 72, or comprises the amino acid sequence of SEQ ID NO: 64, 66, 68, 70 or 72.

24. The polypeptide of any one of claims 21 to 23, wherein the polypeptide comprises an amino acid sequence having at least 80% sequence identity with the amino acid sequence of SEQ ID NO: 48, 50, 52, 54, 56, 58, 60, or 62, or comprises the amino acid sequence of SEQ ID NO: 48, 50, 52, 54, 56, 58, 60, or 62.

25. A nucleic acid molecule comprising a polynucleotide encoding the nanobody or antigen-binding fragment thereof of any one of claims 1 to 14, the antibody or antigen-binding fragment thereof of any one of claims 15 to 16, or the polypeptide of any one of claims 17 to 24.

26. A vector comprising the nucleic acid molecule of claim 25.

27. A cell expressing the nanobody or antigen-binding fragment thereof of any one of claims 1 to 14, the antibody or antigen-binding fragment thereof of any one of claims 15 to 16, or the polypeptide of any one of claims 17 to 24, or comprising the nucleic acid molecule of claim 25, or the vector of claim 26.

28. A pharmaceutical composition comprising the nanobody or antigen-binding fragment thereof of any one of claims 1 to 14, the antibody or antigen-binding fragment thereof of any one of claims to 16, the polypeptide of any one of claims 17 to 24, the nucleic acid of claim 25, the vector of claim 26, or the cell of claim 27, and optionally a pharmaceutically acceptable diluent or carrier.

29. A kit comprising: (a) the nanobody or antigen-binding fragment thereof of any one of claims 1 to 14, the antibody or antigen-binding fragment thereof of any one of claims 15 to 16, or the polypeptide of any one of claims 17 to 24, the nucleic acid of claim 25, the vector of claim 26, the cell of claim 27, or the pharmaceutical composition of claim 28; and (b) a set of instructions.

30. The kit of claim 29, further comprising a detection means.

31. The kit of claim 30, wherein the detection means comprises a secondary antibody.

32. A method of identifying a patient as having an increased risk of a disease or a condition, comprising:
providing a sample from the patient;
determining a level of an afucosylated IgG Fc glycoform or a sialylated IgG Fc glycoform in the sample using the nanobody or antigen-binding fragment thereof of any one of claims 1 to 14, the antibody or antigen-binding fragment thereof of any one of claims to 16, or the polypeptide of any one of claims 17 to 24;
comparing the determined level of the afucosylated IgG Fc glycoform or the sialylated IgG Fc glycoform to a reference level and determining whether the determined level of the afucosylated IgG Fc glycoform or the sialylated IgG Fc glycoform is elevated as compared to the reference level; and
identifying the patient as having an increased risk of developing the disease or condition if the determined level is elevated as compared to the reference level.

33. The method of claim 32, wherein the step of determining the level of the afucosylated IgG Fc glycoform or the sialylated IgG Fc glycoform comprises determining a level of the afucosylated IgG1 Fc glycoform or the sialylated IgG1 Fc glycoform.

34. The method of any one of claims 32 to 33, wherein the step of determining the level of the afucosylated IgG Fc glycoform or the sialylated IgG Fc glycoform comprises determining a level of the IgG1 Fc glycoform afucosylated at Asp297 (EU numbering).

35. The method of any one of claims 32 to 34, wherein the afucosylated IgG Fc glycoform or the sialylated IgG Fc glycoform is an afucosylated IgG Fc glycoform or a sialylated IgG Fc glycoform of an anti-dengue virus (DENV) antibody or an anti-SARS-CoV-2 antibody.

36. The method of any one of claims 32 to 35, wherein the disease or condition is a severe dengue disease caused by a secondary infection by a dengue virus (DENV).

37. The method of claim 36, wherein the severe dengue disease is characterized by a severity level of dengue disease selected from dengue fever (DF), dengue hemorrhagic fever (DHF), and dengue shock syndrome (DSS).

38. The method of any one of claims 32 to 35, wherein the disease or condition is caused by SARS-CoV-2.

39. The method of any one of claims 32 to 38, wherein IgG1 Fc glycoforms comprise at least 3% afucosylated IgG1 Fc glycoforms.

40. The method of any one of claims 32 to 39, wherein IgG1 Fc glycoforms comprise at least 5% afucosylated IgG1 Fc glycoforms.

41. The method of any one of claims 32 to 40, wherein IgG1 Fe glycoforms comprise at least 8% afucosylated IgG1 Fe glycoforms.

42. A method of treating or preventing a virus infection, comprising administering to the patient an effective amount of the nanobody or antigen-binding fragment thereof of any one of claims 1 to 14, the antibody or antigen-binding fragment thereof of any one of claims 15 to 16, the polypeptide of any one of claims 17 to 23, the nucleic acid of claim 25, the vector of claim 26, the cell of claim 27, or the pharmaceutical composition of claim 28.

43. The method of claim 42, wherein the virus infection is caused by a dengue virus or a SARS-CoV-2 virus.

44. The method of claim 42, comprising identifying the patient as having an increased risk of developing severe dengue disease by the method of any one of the preceding claims 32 to 41.

45. The method of any one of claims 42 to 44, further comprising administering to the patient an additional agent or therapy.

46. The method of claim 45, wherein the additional agent or therapy comprises an anti-viral agent.
